(19) United States
(12) Patent Application Publication
(10) Pub. No.: US 2017/0363618 A1
(43) Pub. Date: Dec. 21, 2017

(54) AGE-MODIFIED CELLS AND METHODS FOR MAKING AGE-MODIFIED CELLS

(71) Applicant: MEMORIAL SLOAN-KETTERING CANCER CENTER, NEW YORK, NY (US)

(72) Inventors: Lorenz Studer, New York, NY (US); Daniela Cornacchia, New York, NY (US)

(73) Assignee: MEMORIAL SLOAN-KETTERING CANCER CENTER, NEW YORK, NY (US)

(21) Appl. No.: 15/649,616

(22) Filed: Jul. 13, 2017

Related U.S. Application Data
(63) Continuation of application No. PCT/US2016/013492, filed on Jan. 14, 2016.
(60) Provisional application No. 62/103,471, filed on Jan. 14, 2015.
(60) Provisional application No. 62/109,412, filed on Jan. 29, 2015.
(60) Provisional application No. 62/261,849, filed on Dec. 1, 2015.

(51) Int. Cl. G01N 33/50; C12Q 1/68; C12N 5/074; G01N 33/569

(52) U.S. Cl. CPC G01N 33/5073; G01N 33/5026; G01N 33/56966; C12Q 1/6876; G01N 33/502; C12N 5/0696; C12N 2501/72; C12N 2501/999; G01N 2800/7042; C12Q 2600/154

(57) ABSTRACT

Provided are age-modified cells and method for making age modified cells by reducing or increasing the level of genomic nucleic acid methylation in the cells. The aging and/or maturation process can be accelerated or reduced and controlled for young, aged, mature and/or immature cells, such as a somatic cell, a stem cell, a stem cell-derived somatic cell, including an induced pluripotent stem cell-derived cell, by reducing or increasing the level of genomic nucleic acid methylation in the cells. Methods described by the present disclosure can produce age-appropriate cells from a somatic cell or a stem cell, such as an old cell, young cell, immature cell, and/or a mature cell. Such age-modified cells constitute model systems for the study of late-onset diseases and/or disorders.

### CROSS REFERENCE TO RELATED APPLICATIONS

This application is a continuation of International Patent Application No. PCT/US2016/013492, filed Jan. 14, 2016, which claims priority to U.S. Provisional Application No. 62/103,471 filed Jan. 14, 2015, U.S. Provisional Application No. 62/109,412 filed Jan. 29, 2015, and U.S. Provisional Application No. 62/261,849 filed Dec. 1, 2015, the contents of each of which are hereby incorporated by reference in their entireties, and priority to each of which is claimed.

### 1. TECHNICAL FIELD

The present disclosure relates to methods for accelerating the biological age or aging state of cells by reducing the level of genomic methylation of the cells, wherein said cells can be used both clinically as well as in basic research. The present disclosure is also directed to cells exhibiting one or more chronological markers and methods for inducing such markers in a cell, such as a somatic cell, a stem cell, and/or a stem cell derived somatic cell, including an induced pluripotent stem cell (iPSC)-derived somatic cell. Furthermore, the present disclosure also provides for methods of reversing cellular aging (i.e., cellular rejuvenation) by increasing genomic nucleic acid methylation or other silencing epigenetic marks.

### 2. BACKGROUND OF THE DISCLOSURE

Understanding and reversing the inexorable process of aging represents an ancient dream of mankind. Fortunately, ample evidence shows that the pace of aging can be manipulated, especially in lower organisms, by different interventions. Yet, only few processes are able to reverse an aged state back to a more youthful state, among these, the generation of induced pluripotent stem cell (iPSC). In fact, reprogramming cells to pluripotency not only rewinds the biological clock from a developmental perspective, it also erases several features of age. Evidence has recently been provided showing that re-differentiation of iPSCs into various lineages leaves cells “rejuvenated” by reversing a number of markers associated with cellular aging.

To date, studies that report examples of biological rejuvenation in iPSCs and their differentiated progeny mostly compare phenotypic traits before and after reprogramming. While it has been proposed that pluripotency might restore cellular youth by epigenetic mechanisms, an in depth analysis of this process has not yet been performed. Understanding how rejuvenation is encoded in the genome could provide invaluable knowledge on the molecular determinants of age and open the possibility of devising methods for reprogramming cellular age independently of cellular fate.

Late-onset disorders and/or diseases can occur in a variety of physiological systems. For example, neurodegenerative disorders such as Parkinson's disease (PD) or Alzheimer's disease (AD) are becoming a growing burden to society. Higher life expectancies have led to an explosion of the number of individuals diagnosed with those currently incurable and in many cases untreatable disorders. This trend is expected to escalate, as it is estimated that the afflicted population, individuals over 60 years of age, will represent 21.8% of the total world population reaching 2 billion people by 2050. Lutz et al., Nature 451:716-719 (2008).

Age per se is believed by many to be a significant risk factor for neurodegenerative diseases, and it is estimated that, for example, the cases of AD in the U.S. will more than triple from 4 million in 2010 to nearly 14 million by 2050. Hebert et al., Neurology 80(19):1778-83 (2013). Similar increases in incidence are expected for PD over the next 30 years. Dorsey et al., Neurology 68:384-386 (2007). In parallel, therapies for age related disorders such as AD and PD are being developed at an excruciatingly slow rate. Only symptomatic relief is available, limited in terms of both the symptoms treated and the duration of its effectiveness, highlighting the need for novel preventive and therapeutic approaches.

Late-onset neurodegenerative disorders such as Parkinson's disease (PD) are becoming a growing burden to society due to the gradual increase in life expectancy. The incidence of PD will likely continue to rise, as it is estimated that by 2050 21.8% of the projected world population (approximately 2 billion people) will be over 60 years of age (Lutz et al., Nature 451:716-719 (2008).

The use of induced pluripotent stem cell (iPSC) technology where patient-derived skin cells can be reprogrammed back to a pluripotent state and then further differentiated into disease-relevant cell types presents new opportunities for modeling and potentially treating currently intractable human disorders (Bellin et al., Nat Rev Mol Cell Biol 13, 713-726 (2012). However, there is a concern as to how well iPSC-derived cells can model late-onset diseases where patients do not develop symptoms until later in life, implicating age as a necessary component to disease progression.

Several iPSC studies have demonstrated a loss of particular age-associated features during iPSC induction (reviewed in Freije and López-Otin, Curr Opin Cell Biol 24, 757-764 (2012); Mahmoudi and Brunet, Curr Opin Cell Biol 24, 744-756 (2012)). For instance, there is evidence for changes in age-associated features such as an increase in telomere length (Agarwal et al., Nature 464:292-296 (2010); Marion et al., Cell Stem Cell 141-154 (2009)), mitochondrial fitness (Prigione et al., Stem Cells 721-733 (2010); Suhr et al., PloS One e14095 (2010)) and loss of senescence markers (Lapasset et al., Genes Dev 25: 2248-2253, 2011) in iPSCs derived from old donors, suggesting that rejuvenation takes place during old donor cell reprogramming. In addition to the apparent loss of age-associated features in iPSCs, as compared to their primary somatic cell source, another advantage of using iPS cells in aging of iPS derived cells of the present disclosures is the resulting mature phenotype. In contrast, directed differentiation of human pluripotent stem cells (hPSCs) is known to yield immature, embryonic-like cell types, which lack maturation markers and the ability to display late-onset disease phenotypes. In fact, without induced aging, these immature iPSC-derived cells often require months of in vitro or in vivo maturation to establish robust functional properties of their particular cell type (Liu et al., Curr Opin Cell Biol 24:765-774 (2012); Saha & Jaenisch, Cell Stem Cell 5:584-595 (2009).

Protracted differentiation is thought to reflect the slow timing of human development. For example, human midbrain dopamine (mDA) neurons, the cell type predominantly affected in PD, require months of culture to develop mature physiological behaviors in vitro and months of in vivo maturation to rescue dopamine deficits in animal models of PD (Isacson et al., Trends Neurosci 20:477-482 (1997); Kriks et al., Nature 480:547-551 (2011)). Furthermore, based on the BRAIN-span atlas of the developing human brain (brainspan.org), gene expression data from hPSC-derived neural cells match the transcriptome of first trimester embryos, a stage believed to be too early to model late-onset disorders. These in vitro differentiation data indicated a species-specific intrinsic “clock-like” maturation process that prevented the rapid generation of mature or aged cells posing a major challenge for human iPSC-based modeling of late-onset neurodegenerative disorders such as PD.

A problem in addressing the global aspects of aging and rejuvenation during cell reprogramming and differentiation is the identification of markers that reliably predict the chronological age of the somatic cell donor and the corresponding cellular age of iPSC derivatives.

Induced pluripotent stem cells (iPSCs) have been proposed to be useful for modeling human disease. For example, iPSC technology has been used to study early-onset disorders such as familial dysautonomia or Herpes Simplex encephalitis. (Lee et al., Nat Biotechnol 30:1244-1248 (2012); Lee et al., Nature 461:402-406 (2009); and Lafaille et al., Nature 491:769-773 (2012)). Discovery of the disease mechanisms for both disorders and high throughput drug screening enabled a human iPSC-based disease model on which screened drug candidates could be further tested.

Despite early progress in modeling early-onset genetic disorders, fundamental questions remain as to how well iPSC-based approaches can model late-onset disorders such as Parkinson's disease (PD) given the embryonic nature of iPSC-derived midbrain dopamine (mDA) neurons. (Lee & Studer, Nat Methods 7:25-27 (2010); Saha & Jaenisch, Cell Stem Cell 5:584-595 (2009); and Liu et al., Curr Opin Cell Biol 24:765-774 (2012)). Late-onset disorders such as PD take decades to develop without any signs of the disease at early stages of life. Indeed current studies modeling genetic or sporadic forms of PD using iPSC technology show no observed phenotype or display relatively subtle biochemical or morphological changes without recreating the severe degenerative pathology characteristic of the disease. (Soldner et al., Cell 146:318-331 (2011); Soldner et al., Cell 136:964-977 (2009); Nguyen et al., Cell Stem Cell 8:267-280 (2011); Seibler et al., J Neurosci 31:5970-5976 (2011); and Cooper et al., Sci Transl Med 4:141ra190 (2012)).

The ability to measure and manipulate age in cells differentiated from iPSCs represents a fundamental challenge in pluripotent stem cell research that remains unresolved to date. There has been considerable progress in directing cell fate into the various derivatives of all three germ layers; however, there has been little technology to switch the age of a given cell type on demand from embryonic to neonatal, adult or aged status. This remains a major impediment in the field as illustrated by the persistent failure to generate hPSC-derived adult-like hematopoietic stem cells, fully functional cardiomyocytes, or mature pancreatic islets and the general inability to derive aged cell types that are age-appropriate and/or stage-appropriate for modeling late-onset diseases.

iPSC models of late-onset disorders such as PD do not adequately reflect the severe degenerative pathology of the disease. Thus, new methods to model late-onset neurodegenerative disorders are needed. Specifically, new methods to generate aged cells that more closely resemble the age of the patient using iPSC technology would be very useful in the quest for effective treatments for late-onset diseases, particularly degenerative ones and more specifically neurodegenerative ones.

Additionally, an ability to accelerate maturation of cells would be useful in providing supplies of age-appropriate cells at a rapid pace, whether for research or therapy.

### 3. SUMMARY OF THE DISCLOSURE

Disclosed are methods for producing a cell exhibiting at least one chronological marker, said method comprising reducing the level of methylation of the cell's nucleic acid, for example, genomic DNA.

Such chronological markers include those described herein, and in International Publication No. WO/2014/172507, published Oct. 23, 2014, which is incorporated by reference in its entirety for all purposes.

In certain embodiments, reducing the level of methylation reduces the level of epigenetic repression of gene expression in the cell, for example, de-repression of transposable and repetitive sequences.

In certain embodiments, the methods of the present application comprise contacting a cell with an agent that inhibits or reduces nucleic acid methylation in an amount and for a period of time sufficient to reduce or inhibit the level of nucleic acid methylation in the cell. In some embodiments, the cell can be a stem cell or a somatic cell. In a more particular embodiment, the cell can be an iPSC-derived cell. In a still more particular embodiment the iPSC-derived cell is a neuron. In certain embodiments, the iPSC-derived neuron is a midbrain dopamine neuron (mDA neuron). In certain embodiments, the iPSC-derived mDA neuron is derived from a subject with Parkinson's disease.

In certain embodiments, the somatic cell is produced by a method comprising contacting a stem cell with one or more differentiation factors, wherein said differentiation factors promote the differentiation of said stem cell into said somatic cell. In certain embodiments, the contacting is in vitro or ex vivo.

In certain embodiments, the agent that inhibits or reduces nucleic acid methylation comprises a nucleoside analog of cytidine, for example, zebularine (also known as 1-(β-D-Ribofuranosyl)-2(1H)-pyrimidinone or Pyrimidin-2-one β-D-ribofuranoside).

In certain embodiments, the agent that inhibits or reduces nucleic acid methylation comprises 5-aza-2-deoxycytidine (5-aza-dC; Decitabine) and/or homocysteine and/or the homocysteine metabolite S-adenosyl-1-homocysteine (SAH).

In certain embodiments, the agent that inhibits or reduces nucleic acid methylation comprises 4-Chloro-N-(4-hydroxy-1-naphthalenyl)-3-nitro-benzenesulfonamide (SW155246).

In certain embodiments, the agent that inhibits or reduces nucleic acid methylation comprises (3S,3'S,5aR,5aR,10bR,10′bR,11aS,11′aS)-2,2′,3,3′,5a,5′a,6,6′-octahydro-3,3′-bis(hydroxymethyl)-2,2′-dimethyl-[10b,10′b(11H,11′H)-bi3,11a-epidithio-11aH-pyrazino[1′,2′:1,5]pyrrolo[2,3-b]indole]-1,1′,4,4′-tetrone, (Chaetocin).

In certain embodiments, the agent comprises an inhibitor of a DNA methyltransferase (DNMT) and/or an inhibitor of histone methyltransferase (HMT), for to example, an antibody or fragment thereof that binds to a DNMT and/or an HMT, or an antisense or siRNA molecule that reduced or inhibits expression of a DNMT and/or HMT enzyme. In certain embodiments the DNMT enzyme comprises DNMT1, DNMT3A, DNMT3B, and/or DNMT3L.

In certain embodiments, the agent comprises an inhibitor of a histone methyltransferase, for example, SUV3/9, for example, an antibody or fragment thereof that binds to a histone methyltransferase, or an antisense or siRNA molecule that reduced or inhibits expression of a histone methyltransferase.

In certain embodiments, the agent comprises an inhibitor of a methyl-CpG-binding protein (MeCP2) and/or an inhibitor of a PHD and RING finger domains 1 protein (UHRF1), for example, an antibody or fragment thereof that binds to a MeCP2 and/or UHRF1 protein, or an antisense or siRNA molecule that reduced or inhibits expression of a MeCP2 and/or UHRF1 protein.

In certain embodiments, the reduction in the level of nucleic acid methylation comprises a reduction of methylation at non-coding regions of genomic nucleic acid repetitive elements, for example, LINE1 (L1) elements, LTR (Long terminal repeat) elements, and/or Endogenous Retroviruses (ERV) elements.

In certain embodiments, the reduction in the level of nucleic acid methylation comprises a reduction of methylation at non-coding regions of genomic nucleic acid and a concomitant local hypermethylation of specific genomic nucleic acid promoters.

In certain embodiments, the reduction of epigenetic silencing comprises a reduction in the levels of repressive histone marks, for example, H3K9me3 and/or H3K27me3.

In certain embodiments, the reduction in the level of nucleic acid methylation comprises a reduction in the level of histone protein H1.

In certain embodiments, the reduction in the level of nucleic acid methylation comprises a reduction in the level of heterochromatin marker HP1α.

In certain embodiments, the reduction in the level of nucleic acid methylation comprises a reduction in the level of nuclear morphology marker LaminB1.

In certain embodiments, the reduction in the level of nucleic acid methylation comprises an increase in the level of one or more marker of DNA damage, for example, yH2Ax.

In certain embodiments, the reduction of nucleic acid methylation comprises a reduction in the level and/or rate of methylation at one or more CpG methylation sites.

In certain embodiments, the methods of the present application comprise increasing the level of 5-hydroxy-methyl-cytosine (5hmC) nucleic acid modifications in the cell.

In certain embodiments, the methods of the present application comprise increasing the level or activity of ten-eleven translocation (TET) proteins in the cell, for example, but not limited to, human ten-eleven translocation 1 (TET1).

In certain embodiments, the reduction in nucleic acid methylation described herein generates genomic instability and interferes with normal nuclear functions ranging from transcription to repair, eventually resulting in the loss of homeostasis that defines the aged cellular state.

In certain embodiments, the level of nucleic acid methylation is reduced by a mutation in a DNA methyltransferase (DNMT) and/or a histone methyltransferase (HMT) and/or a methyl-CpG-binding protein (MeCP2) and/or a PHD and RING finger domains 1 protein (UHRF1), for example, a hypomorphic mutation, such as a hypomorphic mutation in DNMT1.

In some embodiments, the at least one chronological marker is selected from the group consisting of an age-associated marker, a maturation-associated marker, and a disease-associated marker.

Disclosed is also a cell exhibiting at least one chronological marker induced by reducing the level of nucleic acid methylation in an amount and for a period of time sufficient to induce said at least one chronological marker.

In certain embodiments, the level of nucleic acid methylation is reduced to a level of between about 10 to 30% of the level of methylation in a cell whose level of methylation was not reduced according to the methods described herein, for example, in an iPSC derived from a somatic cell.

In certain embodiments, the level of nucleic acid methylation is reduced by about 10 to 30% from the level of methylation in a cell whose level of methylation was not reduced according to the methods described herein, for example, in an iPSC derived from a somatic cell.

In some embodiments, the cell is a somatic cell selected from the group consisting of a fibroblast cell, a liver cell, a heart cell, a CNS cell, a PNS cell, a kidney cell, a lung cell, a hematopoietic cell, a pancreatic beta cell, a bone marrow cell, an osteoblast cell, an osteoclast cell, an endothelial cell. In some embodiments, the cell is selected from the group consisting of a neural progenitor, a neuron and a glial cell.

In certain embodiments, the cell is a midbrain dopamine (mDA) neuron cell.

In certain embodiments, the at least one chronological marker is an age-associated marker selected from Table 2 or Table 3, described herein.

Disclosed are also methods for drug screening, comprising contacting an age-modified cell with a candidate drug and detecting an alteration in at least one of the survival, biological activity, morphology or structure of the cell, wherein said age-modified cell exhibits at least one chronological marker induced by reducing the level of genomic nucleic acid methylation in an amount and for a period of time sufficient to induce said at least one chronological marker in said cell. In some embodiments, the screening method comprises contacting an age-modified cell with a candidate drug and to detecting an alteration in at least one of the survival, biological activity, structure or morphology of the cell.

In some embodiments, reducing the level of genomic nucleic acid methylation accelerates the aging and/or maturation of the cell. By controlling this process, the age of a cell can be selected to model late-onset diseases, especially those diseases that otherwise cannot be studied adequately. Thus, the produced cells can be used in variety of applications, including, but not limited, disease modeling, drug screening, and therapeutics.

In other embodiments, the present disclosure provides methods for producing an age-appropriate somatic cell comprising reducing the level of genomic nucleic acid methylation of cells in a culture, wherein said cell culture has at least one first chronological marker signature (e.g., one found in a young or immature cell), and thereby inducing an age-appropriate somatic cell that exhibits at least one second chronological marker signature (e.g., one found in an old or mature cell). In further embodiments, methods of the present disclosure can be applied to produce an age-appropriate somatic cell comprising reducing the level of genomic nucleic acid methylation in a primary somatic cell culture, wherein the primary somatic cell culture has at least one first disease marker signature, wherein the age-appropriate somatic cell culture that is produced exhibits at least one second disease marker signature. The chronological marker signature can comprise one or more chronological markers.

In one embodiment, the neuronal cell is a midbrain dopamine cell. In one embodiment, the neuronal cell culture is a PARKIN neuronal cell. In one embodiment, the neuronal cell culture is a LRRK2 neuronal cell.

In certain embodiments, the present application also provides for methods of increasing the level of methylation of a cell's nucleic acid, for example, genomic DNA. In certain embodiments, the methods produce a cell exhibiting a lower expression level of at least one chronological marker, as described herein, compared to an aged cell or a cell that has not been subjected to the method of increasing nucleic acid methylation.

In certain embodiments, increasing the level of methylation increases the level of epigenetic repression of gene expression in the cell.

In certain embodiments, the methods of the present application comprise contacting a cell with an agent that increases nucleic acid methylation in an amount and for a period of time sufficient to increase the level of nucleic acid methylation in the cell. In some embodiments, the cell can be a stem cell or a somatic cell. In a more particular embodiment, the cell can be an iPSC-derived cell. In a still more particular embodiment the iPSC-derived cell is a neuron. In certain embodiments, the iPSC-derived neuron is a midbrain dopamine neuron (mDA neuron). In certain embodiments, the iPSC-derived mDA neuron is derived from a subject with Parkinson's disease.

In certain embodiments, the cell is contacted with an agent that increases nucleic acid methylation in an amount and for a period of time sufficient to decrease expression of repetitive elements, for example, LINE1 and/or MIR elements.

In certain embodiments, the agent that increases nucleic acid methylation comprises a PIWI protein and/or a PIWI-interacting RNA (piRNA) and/or a somatic transposon protection factor APOBEC3B. In certain embodiments, the agent provides for locus-specific epigenetic silencing through DNA methylation or repressive histone marks.

In certain embodiments, the agent that increases nucleic acid methylation comprises a DNA methyltransferase (DNMT) and/or a histone methyltransferase (HMT) and/or a methyl-CpG-binding protein (MeCP2) and/or a PHD and RING finger domains 1 protein (UHRF1).

In certain embodiments, the agent that increases nucleic acid methylation comprises resveratrol, rapamycin, or a combination thereof.

In certain embodiments, the agent that increases nucleic acid methylation comprises a CRISPR (clustered regularly interspaced short palindromic repeats) nucleic acid comprising a target sequence of interest (for example, as described by Sander and Joung, Nat Biotechnol. 2014 April; 32(4):347-55, which is incorporated by reference in its entirety herein). In certain embodiments, the agent comprises CRISPR fused to a chromatin modifier, for example, a DNMT and/or an HMT protein. In certain embodiments, the agent provides for locus-specific epigenetic silencing through DNA methylation or repressive histone marks.

In certain embodiments, the present disclosure provides for methods for determining the molecular age of a cell comprise determining the ratio of expression levels of one or more Line1 (L1), LTR, and/or ERV repetitive elements to one or more ALU repetitive elements, wherein a ratio greater than 1 is indicative of the cell having an aged or old molecular status.

The presently disclosed subject matter provides for kits for inducing age in a cell, wherein the aged cell expresses one or more chronological markers. In certain embodiments, the kit comprises (a) one or more inhibitors of nucleic acid methylation, and (b) instructions for inducing age in the cell, such that the cell expresses one or more chronological markers of an aged cell, wherein said instructions comprise contacting said cell with said one or more inhibitors of nucleic acid methylation.

Furthermore, the presently disclosed subject matter provides for kits for reducing age in a cell. In certain embodiments, the kit comprises (a) one or more agents that induces or increases nucleic acid methylation, and (b) instructions for reducing age in the cell, such that the expression of one or more chronological markers of an aged cell by the cell is reduced, wherein said instructions comprise contacting said cell with said one or more agents that induces or increases nucleic acid methylation.

The foregoing has outlined broadly the features and technical advantages of the present disclosure in order that the detailed description that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the application as set forth in the appended claims. The novel features which are believed to be characteristic of the application, both as to its organization and method of operation, together with further objects and advantages will be better understood from the following description.

### 4. BRIEF DESCRIPTION OF THE DRAWINGS

FIGS. 1A-1B show decreased DNA methylation and repressive histone marks with age. A) ERRBS results from young, old and iPSC-derived fibroblasts show decrease of DNA methylation in all analyzed genomic regions to levels equivalent to what previously described′. Rates are re-increased in iPSC-derived fibroblasts. Young and old bars depict average and SEM of 4 donors. Preliminary rates in iPSC-fibroblast from 1 young and 1 old donor are shown. B) Western blot quantification of total H3K9me3 and H3K27me3 from young and old primary fibroblasts. Bars show averages and STD of 4 donors, (n=2).

FIGS. 2A-2C show iPSC generation and validation. Images depict representative clones from one young and one old donor. A) Immunofluorescence for pluripotency markers OCT4 (green) and Nanog (red). B) Karyotyping of iPSC clones C) Cell Line authentication through DNA fingerprinting (STR profiling).

FIGS. 3A-3C show distinct transcriptional profiles underlie healthy and premature aging. A) Hierarchical clustering of RNA-Seq data from primary fibroblast of young, old and HGPS donors shows segregation of progeria samples from both young and old healthy cells. B) Venn Diagram of differentially expressed genes in healthy aging (young versus old) and premature aging (young vs. HGPS) shows limited overlap of between normal aging and progeria. C) Differentially enriched GO categories in normal aging (young vs old) and progeria (young vs HGPS). Source: DAVID http://david.abcc.ncifcrf.gov/

FIGS. 4A-4B show A) expression of LINE1 and MIR elements in primary fibroblasts of young and old donors detected by RT-qPCR. Increased expression of the analyzed repetitive elements was detected in old versus young samples. B) Expression of PIWIL2 and APOBEC3B in primary fibroblasts from different age donor groups detected by RNA-Seq confirms minimal expression of PIWI proteins in somatic cells. Moreover, data indicates a gradual decrease in the levels of the somatic transposon protection factor APOBEC3B with age (young>middle-aged>old).

FIGS. 5A-5E show that genome-wide levels of DNA methylation decreases with age. A-D) Global DNAm levels measured by Enhanced Reduced Representation Bisulphite Sequencing (ERRBS) of primary fibroblasts from individuals aged 10-96 years. E) Fluorimetric measurement of global levels of DNAm in primary fibroblasts from four young (10-11 years) and four old (71-96 years) individuals.

FIGS. 6A-6B show that global levels of silencing epigenetic markers and core histones decrease with age. A) Western blot showing global expression levels of the major silencing epigenetic markers H3K9me3, H3K27me3, as well as total histone H3 in primary fibroblasts from young (10-11 years) and old (71-96 years) individuals. B) Densitometric quantification of western blot bands from (A) shows a significant decrease of H3K9me3 (**p=0.0067) and H3K27me3 (**p=0.0015) expression in old versus young cells normalized to γ-Tubulin. Graphs represent results from 3 independent experiments.

FIG. 7 shows that age-dependent loss of DNA methylation is predominant at repetitive and transposable genomic elements. The vast majority of genomic DNA methylation is concentrated at non-coding, repetitive regions such as transposable elements. Accordingly, age-dependent decrease in DNA methylation preferentially affects repetitive elements. ERRBS measurement of DNA methylation rates in young and old fibroblasts shows that 75% of repetitive elements are hypomethylated in old cells compared to young.

FIG. 8 shows age-dependent transcriptional de-regulation of repetitive elements. Loss of DNA methylation at repetitive regions is predicted to lead to transcriptional de-repression of these loci. Accordingly, Total RNA-Seq analysis in young and old fibroblasts reveals an age-dependent differential expression of repetitive transcripts, wherein LINE1 elements appear preferentially upregulated and ALU elements downregulated.

FIG. 9 shows that age-related transcriptional changes of repetitive elements are dependent on repeat class and transcript abundance. Differential expression of repetitive transcript between young and old cells reveals a non-random distribution of age-dependent transcriptional up-versus downregulation. Low abundance elements (30-1000 FPKM), mainly originating from LINE1 (L1), LTR elements and Endogenous Retroviruses (ERVs) are preferentially upregualted in cells from old individuals, whereas high abundance transcripts (10,000-100,000 FPKM), mostly originating from ALU elements, appear downregulated.

FIG. 10 shows toxicity assay results for all 6 compounds used to treat young and old fibroblasts, as described by Example 13. Blue/Black curve indicates day 3 test; red/grey curve indicates day 7 test. Blue box indicates range generally used in prior experiments; red box indicates range used in Example 13. Toxicity test indicated that at day 7 compounds had similar toxicity levels as at day 3, indicating that the full toxic effects that are seen at day 10 occur between days 7-10.

FIG. 11 shows the effect of 3-day culture of young (348) and old (204) fibroblasts with resveratrol and rapamycin on expression levels of histone protein H1, heterochromatin marker HP1α, H3K9me3, H3K27me3, nuclear morphology marker LaminB1 (markers of young cellular age), and yH2Ax (marker of old cellular age). UT refers to untreated, and C3, C2, and C1 are increasing concentrations of drug compound (C3 being lowest). Graphs are data from duplicate wells, and are normalized to the untreated intensities. Resveratrol increased levels of all markers.

FIGS. 12A-12C show primary fibroblasts from young and old individuals that were untreated control (Con) or treated for three days with 6.25 μM (low), 12.5 μM (med), and 25 μM (high) of Resveratrol (Chen and Guarente, Trends Mol Med 2007), as described by Example 13. In contrast to the treatment with SW155246 (see FIG. 14), treatment with Resveratrol significantly reverses markers of cellular age by increasing levels of (A) HP1a and (B) H1, and decreasing levels of (C) γH2AX.

FIG. 13 shows the effect of 3-day culture of young (348) and old (204) fibroblasts with Decitabine, Zebularine, SW155246, and Chaetocin on expression levels of histone protein H1, heterochromatin marker HP1α, H3K9me3, H3K27me3, nuclear morphology marker LaminB1 (markers of young cellular age), and yH2Ax (marker of old cellular age). UT refers to untreated, and C3, C2, and C1 are increasing concentrations of drug compound (C3 being lowest). High levels of γH2Ax indicate possible toxicity.

FIGS. 14A-14D show primary fibroblasts from young and old individuals that were untreated control (Con) or treated for three days with 0.8 μM (low), 1.6 μM (med) and 3.2 μM (high) of the selective DNMT1 inhibitor SW155246. (Kilgore et al., JBC 2013), as described by Example 13. Genomic aging markers HP1α, H1 and γH2AX were subsequently quantified by immunofluorescence. High levels of HP1α and H1 are indicators of chromatin compaction and therefore of a younger state, whereas γH2AX marks DNA damage sites and is increased with age. Treatment with SW155246 significantly induces markers of cellular age by decreasing levels of (A) HP1α and (B, D) H1 and increasing levels of (C) γH2AX.

FIG. 15 shows the effect of a 10-day culture of young (348) and old (204) fibroblasts with Decitabine, Zebularine, SW155246, Chaetocin, resveratrol or rapamycin on cell survival as measured by cell number counts. Cell numbers are calculated as a mean of 8 wells.

FIGS. 16A-16D show the effect of a 3-day culture of young (348) and old (204) fibroblasts with Decitabine, Zebularine, SW155246, or Chaetocin on global DNA levels of 5-mC methylation. (A) Levels of 5-mC did not consistently go down with treatment when treated with SW155246, a selective DNMT1 inhibitor. (B) Chaetocin, a SUV3/9 inhibitor, caused levels of 5-mC methylation to increase. (C,D) Decitabine and Zebularine, both DNMT1/3a&b inhibitors, caused global levels of 5-mC methylation to decrease.

FIG. 17 shows a description of the cell culture protocol used to differentiate iPSCs into midbrain dopamine neurons (mDA). iPSCs were cultured according to Kriks et al., Nature. 2011 Nov. 6; 480(7378):547-51 and Miller et al., Cell Stem Cell. 2013 Dec. 5; 13(6):691-705, wherein the protocols were modified by culturing the iPSCs for 12-24 hours (culture days −0 to −2) before differentiation of the cells into mDA, and further, wherein the wingless (Wnt) signaling inhibitor XAV939 was added to the cell culture from days 0-2 when differentiating the iPSCs into mDA. The mDA cells were subjected to passage at days 13 and/or 15 and 30 of culture, wherein the cells were filtered and plated at a lower density in the day 30 passage. DAPT (N-[(3,5-Difluorophenyl)acetyl]-L-alanyl-2-phenyl]glycine-1,1-dimethylethyl ester) was added to the culture beginning at day 11, and the cells were treated with mitomycin C for 1 hour at day 32. Cells.

FIG. 18 shows the effect of the mitichondrial stressors rotenone and carbonilcyanide p-triflouromethoxyphenylhydrazone (FCCP) on oxygen consumption of iPSC-derived mDA after culture for 65 and 30 days. Undifferentiated iPSCs (culture day 0) were used as controls. mDA cultured to 65 days exhibited greater oxygen consumption under the stressed conditions compared to the 30 day cultured mDA and undifferentiated iPSC controls.

### 5. DETAILED DESCRIPTION

The present disclosure relates to methods for accelerating the maturation of cells by reducing the level of genomic methylation of the cells, and cells produced by such methods and compositions comprising such cells. The cells produced according to the methods described herein can be used for cell therapy for the treatment of disease, such as Parkinson's disease, and to in vitro cell-based systems for modeling of disorders and/or diseases, in particular late-onset disorders and/or diseases. More specifically, provided herein are somatic cells, and methods for producing such cells, which may be primary cells (as defined below) or may be derived from undifferentiated (stem) cells, such as induced pluripotent stem cells (iPSCs), embryonic stem cells or stem cells collected from human or animal subjects. The somatic cells exhibit one or more markers that are characteristic of cellular age, maturation, and/or disease as can be confirmed by detecting one or more intracellular or morphologic markers and/or be detecting the absence of one or more intracellular markers including one or more markers that constitute an intracellular chronological marker signature. In certain embodiments the methods of reducing the level of genomic nucleic acid methylation in cells, for example, iPSCs, can be performed before or after differentiation to a desired cell type.

For purposes of clarity of disclosure and not by way of limitation, the detailed description is divided into the following subsections:
5.1. Definitions;
5.2. Methods for Inducing Aging;
5.3. Parkinson's Disease Modeling;
5.4. Method of Treatment;
5.5 Method of Screening Therapeutic Compounds;
5.6. Method of Determining Molecular Age;
5.7 Methods for Reducing Aging; and
5.8. Kits.

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

An “individual” or “subject” or “patient” as described herein is a vertebrate, such as a human or non-human animal, for example, a mammal. Mammals include, but are not limited to, humans, primates, farm animals, sport animals, rodents and pets. Nonlimiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “young” in reference to an individual refers to an early chronological age, which for humans refers to age in years. The term “young” in reference to a cell refers to a cell displaying a marker signature of cells isolated from young donors (for example, but not limited to, the markers described by Table 1), for example, a cell state such as an immature cell, such as a young iPSC-derived somatic cell, i.e., a cell displaying a marker signature of cells isolated from young donors regardless of the age of the donor of the original primary cell that gave rise to the iPSC. This is to be contrasted with “old” iPSC-derived or indeed any somatic cell which displays a marker signature of cells isolated from old donors. An example of an old iPSC derived somatic cell is that produced when the level of genomic nucleic acid methylation in an iPSC-derived somatic cell is reduced (again, regardless of the age of the donor of the primary cell that gave rise to the iPSC) following reprogramming. A young cell may also refer to a population of “young cells” such as young primary cells derived from a donor of young chronological age as in “young primary fibroblasts.”

As used herein, the term “old” in reference to an individual refers to chronological age, which for humans refers to age in years. The term “old” in reference to a cell refers to a cell displaying a marker signature of cells isolated from old donors (for example, but not limited to, the markers described by Tables 1-3), for example, a cell state wherein the cell expresses one or more chronological markers associated with aged cells, or primary somatic cells from old donors. An old cell may also refer to a population of “old cells” such as old primary cells derived from a donor of old chronological age as in “old primary fibroblasts.”

With respect to stem-cell derived somatic cells, the effects of reducing the level of genomic nucleic acid methylation include without limitation (depending on the type of cells) induction of age-related phenotypes affecting nuclear morphology and expression of nuclear organization proteins as well as markers of heterochromatin, DNA damage and reactive oxygen species, dendrite degeneration, the formation of age-associated neuromelanin, AKT deregulation, selective reduction in the number of TH-positive neurons, and ultrastructural evidence of mitochondrial swelling and inclusion bodies.

As used herein, the term “donor individual” or “donor” refers to any organism, human or non-human, from which cells were obtained to provide a primary cell culture. The donor individual may be of any age, and may be non-diseased or diseased. The donor may provide cells for use in the present methods, by providing biological samples, including a biopsy, a skin biopsy, blood cells, and the like.

The term “disease,” as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent or acquired defects of the organism (as genetic or epigenetic anomalies); and/or iv) combinations of these factors.

As used herein, the term “late-onset disease” refers to a disease or medical condition of a patient manifesting as a clinical condition in middle age and old age patients. Such that a late-onset disease may include but not limited to degenerative, such as neurodegenerative diseases, such as Parkinson's disease (PD), amyotrophic lateral sclerosis, Alzheimer's, Huntington's disease, and diseases of other lineages including cardiac hypertrophy, cardiac fibrosis, Type II diabetes, age-related macular degeneration, cancers, including for example breast cancers, colon cancers, and ovarian cancers, familial adenomatous polyposis (FAP), heart disease, and the like. See, Wright et al., Trends Genet 19:97-106 (2003), incorporated by reference.

As used herein, the term “cell culture” refers to any in vitro culture of cells in an artificial medium for research or medical treatment. Included within this term are continuous cell lines (e.g., with an immortal phenotype), primary cell cultures, finite cell lines (e.g., non-transformed cells), and any other cell population maintained in vitro, including oocytes and embryos.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

The term “deficient” as used herein refers to a cell which either does not express the mRNA of a gene, a protein product of a gene, or both (i.e., devoid of such expressions), or expresses them at a reduced level.

As used herein, the term “neuronal maturation medium” or “BAGCT medium” refers to a culture medium comprising N2 medium, further comprising brain-derived neurotrophic factor (BDNF), ascorbic acid (AA), glial cell line-derived neurotrophic factor, dibutyryl cAMP and transforming growth factor type β3 for differentiating midbrain fate FOXA2/LMX1A+ dopamine (DA) neurons.

As used herein, the terms “purified,” “to purify,” “purification,” “isolated,” “to isolate,” “isolation,” and grammatical equivalents thereof as used herein, refer to the reduction in the amount of at least one contaminant from a sample. For example, a cell type is purified by at least 10%, preferably by at least 30%, more preferably by at least 50%, yet more preferably by at least 75%, and most preferably by at least 90%, reduction in the amount of undesirable cell types. Thus purification of a cell type results in “enrichment,” i.e., an increase in the amount, of the cell type in the cell culture.

As used herein, the term “differentiation agent” or “differentiation inducing compound” refers to a substance, which can be a biological molecule or a small molecule or a mixture of substances which has the property of causing a stem cell to commit to a cellular pathway leading to a somatic cell. For example, such inducing compounds may include, but are not limited to, Wnt activators or SMAD inhibitors.

As used herein, the term “sonic hedgehog protein or SHH” refers to one of three proteins in the mammalian signaling pathway family called hedgehog. SHH is believed to play a role in regulating vertebrate organogenesis, such as the growth of digits on limbs and organization of the brain. Sonic hedgehog protein is thus a morphogen that diffuses to form a concentration gradient and has different effects on the cells of the developing embryo depending on its concentration. SHH may also control cell division of adult stem cells and has been implicated in development of some cancers.

As used herein, the term “Small Mothers against Decapentaplegic” or “SMAD” are intracellular proteins that transduce extracellular signals from transforming growth factor beta ligands to the nucleus where they activate downstream gene transcription and are members of a class of signaling molecules capable of modulating directed differentiation of stem cells.

As used herein, the term “contacting” refers to exposing the cell to a compound or substance in a manner and/or location that will allow the compound or substance to exert its activity on the cell, for example, by touching the cell. Contacting may be accomplished using any suitable method and may be extracellular or intracellular. For example, in one embodiment, contacting is by introducing the compound/substance intracellularly either as such or by genetically modifying the cell, such that it expresses the compound or substance. Contacting can be achieved by a variety of methods, including exposing cells to a molecule or to a vehicle containing a molecule, delivering a polynucleotide encoding for a polypeptide to the cells through transfection. Contacting may also be accomplished by adding the compound or substance to a culture of the cells so that the contacting occurs on the outer cell membrane. Contacting may also be accomplished within a given cell by the production of a recombinant protein within a cell.

As used herein, the terms “reprogramming,” “reprogrammed” refer to the conversion of “primary cells” or “primary differentiated cells” or “primary somatic cells” into undifferentiated cells (i.e., cells that has not yet developed into a specialized cell type), such as induced pluripotent stem (iPS) cells. For example, a somatic cell culture of primary cells, (e.g., for example, primary fibroblasts isolated from donors of certain ages or primary fibroblasts isolated from patients having a disease, such as Parkinson's disease (PD), e.g., PD fibroblasts, etc.), including cell lines, may be reprogrammed into induced pluripotent stem cells. Further, an age-related marker signature appearing in the primary somatic cell culture is then altered in the reprogrammed, undifferentiated cells. In some instances, disease marker signatures appearing in the differentiated somatic cell cultures (i.e., for example, PD marker signatures) may be absent in the converted undifferentiated cells, however the exact signature may differ between iPS cells produced from different primary somatic cell donors. Primary cells may be obtained from any source, such as from donors, i.e. a biopsy, a skin biopsy, a blood draw, and the like, cell lines, and the like.

As used herein, the term “differentiated” refers to a cell, for example an unspecialized embryonic cell, that has undergone a process whereby the cell acquires the features of a specialized cell such as a heart, liver, or muscle cell. Differentiation is controlled by the interaction of a cell's genes with the physical and chemical conditions outside the cell, usually through signaling pathways involving proteins embedded in the cell surface. In certain embodiments, a “differentiated” somatic cell refers to a cell having a more committed cell type characteristic, such as a marker signature characteristic of its type. In certain embodiments, a “differentiated” iPSC-derived somatic cell refers to a cell that has at least one marker signature not present in the iPSC, for example, a marker signature of a specialized cell.

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein). In certain embodiments, “inducing differentiation” refers to a process initiated by compounds that act as differentiation agents, including, but not limited to, Wnt inhibitors and/or activators, sonic hedgehog proteins and/or activators, and/or SMAD inhibitor molecules. Such agents trigger or promote the largely genetically controlled differentiation process which converts an undifferentiated cell (such as an embryonic stem cell, an induced pluripotent stem cell, a primary stem cell etc.), to a committed somatic phenotype, that of a specialized cell having a more distinct form and function, which may or may not admit further differentiation. For example, induced pluripotent stem cells may be converted into iPSC-derived fibroblasts or iPSC-derived neurons, including without limitation neuron with a specific type of junction, specific range of electrical transmission rate, specific types of neurochemical production and/or secretion, etc.

As used herein, the term “aging,” in reference to a cell or cell population, refers to any stage during the progression from expression of a young marker signature towards an old marker signature. One example of aging is the natural aging process in a cell characterized by molecular and morphological markers associated with an aged cell, such as genomic instability, telomere shortening, loss of proteostasis, loss of heterochromatin and altered gene transcription, mitochondrial dysfunction, cellular senescence, and stem cell exhaustion. An example of induced aging is shown herein after reducing genomic nucleic acid methylation levels of young cells in a culture. Aging can also encompass maturation, whereby additional molecular, physical and functional properties of an adult cell (including a chronological marker signature) are expressed.

As used herein, the term “accelerated cellular aging” refers to the establishment of an age-related marker signature in an iPSC-derived somatic cell characterizing a different age relative to what is created by differentiation alone, such that an “aged” iPSC-derived somatic cell is created. For example, this process can be mediated by reducing the level of genomic nucleic acid methylation in the iPSC-derived somatic cell, for example, by introducing an inhibitor of methylation into the cell, for example, an inhibitor of DNA methyltransferase activity such as an antisense molecule, siRNA molecule or antibody that binds to the enzyme. As described herein, this process induces a reprogrammed/differentiated iPSC-derived somatic cell into an aged iPSC-derived somatic cell.

As used herein, the term “directed differentiation” refers to a manipulation of stem cell culture conditions to induce differentiation into a particular (for example, desired) cell type, such as neuronal precursors. As used herein, the term “directed differentiation” in reference to a stem cell refers to the use of small molecules, growth factor proteins, and other growth conditions to promote the transition of a stem cell from the pluripotent state into a more mature or specialized cell fate (e.g. neuron precursors, neurons, etc.).

As used herein, the term “chronological marker signature” refers to any intracellular structure that is characteristic of the specific age of the donor individual or of a cell such that it is sufficient to determine that state. A single marker signature may be sufficient to characterize the age of primary cells from a donor or the age phenotype of a cell (notably a cell differentiated from a stem cell that has no age characteristics of the donor individual or that has lost them such as during reprogramming and subsequent differentiation) wherein the age-related phenotype has been induced, or a profile of a plurality of different marker signatures may be evaluated to characterize the age of a donor or indeed any other cell.

As used herein, the term “marker” refers to a molecular or morphologic trait characteristic of a state of a cell and therefore useful, alone or in combination with other markers, in indicating that state A “marker” can be a “chronological marker,” which includes “age-related markers” and “maturation-related markers.” “Markers” can also be “disease related markers,” which include “late-onset disease markers.” If a single marker (or combination of markers) is sufficient in indicating the state of a cell, it constitutes a marker signature, as further explained below. In certain embodiments, a “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “age-related marker signature” refers to any chronological marker signature (comprising one or more markers) that is characteristic of the natural aging process. A single age-related marker signature may be sufficient to characterize the age of primary cells from a donor or the phenotypic stage of cells wherein an age phenotype has been induced, or a profile of a plurality of different marker signatures may be evaluated to characterize the age of primary cells from a donor or the phenotypic stage of cells wherein an age phenotype has been induced or the phenotypic age of a cell.

As used herein, the term “maturation-related marker signature” refers to any chronological marker signature that is characteristic of the natural maturation process. A single maturation-related marker signature may be sufficient to characterize the maturation stage of primary cells or the phenotypic stage of cells wherein an age phenotype has been induced, or a profile of a plurality of different marker signature maybe evaluated to characterize the maturation stage of primary cells or the phenotypic stage of cells wherein an age phenotype has been induced.

As used herein, the term “disease-related marker signature” refers to any cellular structure (molecular or morphologic) that is characteristic of a specific disease. A single marker signature may be sufficient to characterize a disease, or a profile of a plurality of different marker signatures may need to be evaluated to characterize a disease state.

As used herein, the term “cell” refers to a single cell as well as to a population of (i.e., more than one) cells. The population may be a homogeneous population comprising one cell type, such as a population of neurons or a population of undifferentiated embryonic stem cells. Alternatively, the population may comprise more than one cell type, for example a mixed neural cell population comprising neurons and glial cells. It is not meant to limit the number of cells in a population, for example, a mixed population of cells may comprise at least one differentiated cell. In one embodiment, a mixed population may comprise at least one differentiated cell and at least one stem cell. In the present disclosure, there is no limit on the number of cell types that a cell population may comprise.

As used herein, the terms “primary cell” or “primary somatic cell” refers to any cell in the body other than gametes (egg or sperm), sometimes referred to as “adult” cells, which can be reprogrammed for generating an undifferentiated iPSC in accordance with the methods disclosed herein and/or under the appropriate conditions, i.e. when contacted with a proper growth factor, compound, extracellular signal, intracellular signal, transfected with reprogramming genes (factors), etc. For example, a primary cell (culture) comprises a fibroblast cell, differentiated primary somatic cell, stem cell lines, and the like. In some embodiments, primary cells are isolated from patients. In some embodiments, primary cells are cell lines. In some embodiments, primary cells are stem cell lines. In some embodiments, primary cells are embryonic stem cells. In some embodiments, primary cells are isolated from sources such as from healthy volunteers, from patients, from patients having a particular disease or medical condition, regardless of clinical manifestation, i.e. patients having a certain genotype or phenotype. In some embodiments, primary cells are isolated from mammals. In some embodiments, primary cells are isolated from animals.

A “somatic cell” refers to any cell of an organism, which is a constituent unit of a tissue, skin, bone, blood, or organ, other than a gamete, germ cell, gametocyte, or undifferentiated stem cell. Somatic cells include progenitor cells and terminally differentiated cells. Such somatic cells include, but are not limited to, neurons, fibroblast cells, cardiomyocyte cells, epithelial cells, neuroendocrine cells, pancreatic cells, astrocytes, hematopoietic cells, midbrain dopamine neurons, motoneurons, and/or cortical neurons. As used herein, the term “neural cell culture” refers to a cell culture of neurons and/or glia wherein the cells display characteristics of cells of the central and/or peripheral nervous systems.

As used herein, the term “permissive state” in reference to a somatic cell (iPSC-derived or not) refers to a cell wherein the level of genomic nucleic acid methylation has been reduced, and consequently capable of expressing mature or old “age” markers if the cell is capable of aging and/or to reveal a disease phenotype if present. For example, reducing the level of genomic nucleic acid methylation induces iPSC-derived somatic cells to reach a permissive state, enabling modeling of late-onset diseases.

As used herein, the term “stem cell” refers to a cell that is totipotent or pluripotent or multipotent and is capable of differentiating into one or more different cell types, such as embryonic stem cells or stem cells isolated from organs, for example, mesenchymal or skin stem cells or induced pluripotent stem cells.

As used herein, the term “embryonic stem cell” refers to a primitive (undifferentiated) cell that is derived from preimplantation-stage embryo, embryo, placenta or umbilical cord capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “induced pluripotent stem cell” or “iPSC” refers to a type of pluripotent stem cell that is similar to an embryonic stem cell but is created when somatic (e.g., adult) cells are reprogrammed to enter an embryonic stem cell-like state by being forced to express factors important for maintaining the “stemness” of embryonic stem cells (ESCs), i.e., their ability to be led to commit to different differentiation pathways. Such factors can include certain embryonic genes (such as a OCT4, SOX2, and KLF4 transgenes) (see, for example, Takahashi and Yamanaka Cell 126, 663-676 (2006), herein incorporated by reference) which are introduced into a somatic cell.

As used herein, the term “progenitor” in reference to a cell refers to an intermediate cell stage wherein said cell is no longer a pluripotent stem cell and is also not yet a fully committed cell. Progenitor cells in this disclosure are included within somatic cells.

Stem cells according to the present disclosure can be “totipotent” stem cells, “pluripotent” stem cells, and/or “multipotent” stem cells. As used herein, the term “totipotent” refers to an ability of a cell to differentiate into any type of cell in a differentiated organism, as well as into a cell of extra embryonic materials such as placenta. As used herein, the term “pluripotent” refers to a cell or cell line that is capable of differentiating into any differentiated cell type, for example, an ability to develop into the three developmental germ layers of the organism including endoderm, mesoderm, and ectoderm. As used herein, the term “multipotent” refers to a cell or cell line that is capable of differentiating into at least two differentiated cell types.

Mouse iPSCs were reported in 2006 (Takahashi and Yamanaka, Cell 126:663-676 (2006)), and human iPSCs were reported in late 2007 (Takahashi et al. Cell. 2007 Nov. 30; 131(5):861-72). Mouse iPSCs demonstrate important characteristics of pluripotent stem cells, including the expression of stem cell markers. Human and animal iPSCs also express stem cell markers and are capable of generating cells characteristic of all three germ layers. Unlike an embryonic stem cell, an iPSC is formed artificially by the introduction of certain embryonic genes into a somatic cell (such as an OCT4, SOX2, and KLF4 transgenes). See, for example, Takahashi and Yamanaka, Cell 126:663-676 (2006) and Agarwal et al., Nature 292-296 (2010). iPSC can be produced from adult human skin cells, or fibroblast cells, which are transfected with one or more genes such as, for example, one or more of OCT4, SOX2, NANOG, LIN28, and/or KLF4. See, Yu et al., Science 324:797-801 (2009). Alternatively, they can be produced from other types of somatic cells, such as blood or keratinocytes.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated embryo), or fluids using any manipulation, such as, without limitation, single cell isolation, cultured in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

As used herein, the term “age-appropriate iPSC-derived somatic cell” refers to any cell that was derived from the differentiating of a first stem cell (which in turn may have come from the reprogramming of a primary somatic cell) followed by a reduction in the level of genomic nucleic acid methylation. Age-appropriate iPSC-derived somatic cells are not necessarily characterized by a chronological marker signature of the first cell from which they were derived and may display an immature, young, mature or old age-related marker signature. These cells are “age-appropriate” in that they display markers of a cell age that is appropriate for their intended use. For example, a mature but not old cell is appropriate for establishing models of cells of adult but not old individuals.

As used herein the term, “in vitro” refers to an artificial environment and to processes or reactions that occur within an artificial environment. In vitro environments include, but are not limited to, test tubes and cell cultures.

As used herein the term, “in vivo” refers to the natural environment (e.g., in an animal) and to processes or reactions that occur within a natural environment, such as embryonic development, cell differentiation, neural tube formation, etc.

As used herein, the term “cultured cells” generally refer to cells that are maintained in vitro. Cultured cells include “cell lines” and “primary cultured cells.” The term “cell culture” refers to any in vitro culture of cells. Included within this term are continuous cell lines (e.g., with an immortal phenotype), primary cell cultures, finite cell lines (e.g., non-transformed cells), and any other cell population (notably neurons) maintained in vitro, including embryos, pluripotent stem cells.

The term “small molecule” as used herein, refers to any organic molecule of a size comparable to those organic molecules generally used in pharmaceuticals. The term excludes biological macromolecules (e.g., peptides, proteins, nucleic acids, etc.). Preferred small molecules range in size from approximately 10 Da up to about 5000 Da, more preferably up to 2000 Da, and most preferably up to about 1000 Da.

As used herein, the term “expressing” in relation to a gene or protein refers to making an mRNA or protein which can be observed using assays such as microarray assays, antibody staining assays, and the like.

### 5.2 Methods for Inducing Aging

Conventional reprogramming of somatic cells to induced pluripotent stem cells (iPSCs) resets their phenotype back to an embryonic age, and thus presents a significant hurdle for modeling late-onset disorders. In addition, stem cells collected from human subjects and somatic cells derived from such stem cells are also generally devoid of age and often also of disease markers in the case of late-onset diseases. As described herein, methods are disclosed for inducing appropriate chronological marker signatures in stem cell-derived somatic cells, including without limitation human iPSC-derived lineages, and thus generating age-appropriate cell cultures suitable as disease models.

In certain embodiments, such disease models can be developed by inducing aging chronologic marker signatures in somatic cells (not necessarily derived by induced differentiation of stem cells) that express a “young” marker signature. This strategy can be applied to cell cultures derived from a patient with a late-onset disease and/or disorder including, but not limited to, a neurodegenerative disease, such as Alzheimer's disease (AD) or Parkinson's disease (PD), a cardiomyocyte-related disease, a pancreatic disease, and/or a hematopoietic disease, to derive age-appropriate cell cultures that more accurately represent patient age and thus the disease state.

Methods of the present disclosure can also be applied to cells utilized for drug screening, or any other experiment relevant to late-onset disease, using the aged cells described herein, for example, aged iPSC derived cells. Any drug screening methods known in the art can be used with the cells described herein. For example, methods of screening for drugs for treating ALS using iPSC derived cells (which have not been aged) is described by Yang Y. M. et al., 2013 Cell Stem Cell 12, 713-726 (which is incorporated by reference in its entirety).

In certain embodiments, the present disclosure provides for methods of inducing accelerated aging and/or maturation of cells in culture by reducing the level of genomic nucleic acid methylation. In certain embodiments, reducing the level of genomic nucleic acid methylation in iPSC-derived cell cultures (e.g., iPS cell-derived fibroblasts and iPS cell-derived neurons) induces one or more chronological markers that constitute one or more chronological marker signatures and other characteristics of an age-appropriate cell, such as a mature cell and/or an old cell.

In some embodiments, the present disclosure relates to cells with a long lifespan in vivo which are typically not quickly replenished, if at all, once damaged or diseased, such as neurons and cardiomyocytes, and to methods to obtain such cells at an aged state. These cells, when cultured in vitro, usually need long culture times to exhibit aging and/or maturation markers that represent their counterparts in vivo. Such procedures, when available, are protracted and have high cost. In some embodiments, the present disclosure relates to methods of reducing the level of genomic nucleic acid methylation to accelerate their maturation or aging, or both, and thereby to provide an age-appropriate cell. In some embodiments, these cells can be used to model late-onset diseases, such as neurodegenerative diseases, atherosclerosis and other chronic metabolic diseases.

In some embodiments, the present disclosure relates to controlled maturation and/or aging of mammalian cells in a cell culture by reducing the level of genomic nucleic acid methylation. Used alone, or in combination with other reagents (such as cell differentiation protocols for iPSC cells), methods by the present disclosure grant the ability to accelerate cell maturation and/or aging at a controlled speed which can be manipulated by adjusting the level of genomic nucleic acid methylation. For example, the maturation and/or aging of cells by methods of the present disclosure can be slowed by reducing the dose, concentration and/or exposure frequency of a methylation inhibitor exposed to the cells, or reducing the time of exposure of the inhibitor. Alternatively, methyltransferase enzymatic activity can be reduced or inhibited by introducing an inhibitory factor of the protein (e.g., RNA silencing, RNAi, antisence molecule, antibody (for example, a monoclonal antibody (mAb)) or fragment thereof specific for the protein, etc.). The matured cells can be subjected to additional procedures or be used in experiments, for example, methods of screening for therapeutic compounds, or in cell therapy, as described herein.

Within certain embodiments, the present disclosure provides methods for inducing accelerated aging in an iPSC-derived cell, such as an iPSC-derived somatic cell, which methods include reducing the level of genomic nucleic acid methylation, thereby inducing in the cell one or more chronological marker signatures and/or other age-related characteristics. Within some aspects of these embodiments, a marker signature and/or characteristic is associated with aging and/or one or more disease phenotype.

For example, cell type-specific chronological marker signatures can include, but are not limited to, a combination of one or more disease or chronological markers presented in Tables 1 and 2 and/or the absence of one or more of the disease or chronological markers presented in Tables 1 and 2. Cell type-specific characteristics can include, but are not limited to, one or more phenotypes such as, for example, neuromelanin accumulation in aged iPSC-derived dopamine neurons. Disease phenotypes (related to Parkinson's disease) in neurons include, but are not limited to, pronounced dendrite degeneration, progressive loss of tyrosine-hydroxylase (TH) expression, and/or enlarged mitochondria or Lewy body-precursor inclusions. Hypomethylation-induced aging of Parkinson's disease (PD)-iPSC-derived dopamine neurons can induce disease phenotypes that may be based upon genetic susceptibility.

Disease phenotypes may, in some instances, be based upon aging and/or genetic susceptibility. Accordingly, the present disclosure provides methods for inducing aging to examine late-onset disease and/or disorders in age-appropriate iPSC-based cell culture models, which are characterized by the induction and display of one or more chronological marker signatures, and optionally one or more disease signatures (including for example genetic pre-disposition).

The methods of the present invention can be applied to production of aged cells or mature cells from somatic cells (whether iPSC-derived or primary cells), from stem cells or from fully differentiated or partially differentiated cells.

The present disclosure also provides: (1) methods for inducing maturation or aging in a cell, including a somatic, a stem cell, iPSC and/or a stem cell- or iPSC-derived somatic cell displaying a marker signature of a “young” or of an “immature” cell; (2) methods for using induced aging in cell cultures (whether somatic or stem cell cultures, iPSC-derived or primary, or cells in the course of differentiation) to study chronological effects in late-onset diseases and/or disorders, such as Parkinson's disease (PD), in cultures of age-appropriate cells; and (3) iPSC-derived cells, including age-appropriate iPSC-derived cells, which produce one or more chronological markers or do not produce one or more chronological markers, the presence or absence of which chronological markers is characteristic of a chronological marker signature and/or a particular cellular phenotype (see, Tables 1 and 2).

TABLE 1
Summary of Chronological Marker Signature Phenotypes

| | “Young” | “Old” | “Young” | “Old” mDA | “Old” PD |
|---|---|---|---|---|---|
| Phenotypes | Fibroblast | Fibroblast | mDA neuron | neuron | mDA neuron |
| Nuclear | Uniform | Folding/blebbing/ | Folding | More | Same |
| shape | | expansion | | folding/sporadic | |
| | | | | blebbing | |
| LAP2α | Up | Down | Up | V: Mostly | Same |
| | | | | unchanged | |
| | | | | EA: Down | |
| H3K9me3 | Up | Down | Up | V: Mostly | Same |
| | | | | unchanged | |
| | | | | EA: Down | |
| HP1γ | Up | Down | Up | V: Mostly | Same |
| | | | | unchanged | |
| | | | | EA: Down | |
| γH2AX foci | Rare | Frequent | Moderate | Frequent, | Frequent |
| | | | | larger | (already larger) |
| mtROS | Low | High | Moderate | High | |
| Senescence | Rare | Frequent | NA | NA | NA |
| Apoptosis | NA | NA | Moderate | High | Higher |
| Neurites | NA | NA | Long | Short | Shorter |
| P-Akt | Moderate | Increased | Moderate | Increased | Decreased |

Some embodiments of the present disclosure provide methods for the use of a set of cellular markers that closely correlate with the chronological age of a donor cell, such as a donor fibroblast, which cellular markers include, but are not limited to, markers of nuclear organization, heterochromatin, DNA damage, and mitochondrial stress. Without being bound by theory, it is believed that one or more age-associated markers, associated with the age of the cell of the original donor, are lost upon reprogramming. Moreover, certain features of aging are not reacquired by iPSC-derived lineages upon differentiation. Thus, reducing the level of genomic nucleic acid methylation in apparently healthy cells, induces one or more age-associated markers that define the age of the donor cell prior to iPSC induction.

Thus, the present disclosure provides methods for inducing aging in a cell, which aging mimics several aspects of normal aging in iPSC-derived lineages but is accelerated. The iPSC-derived cells include but are not limited to fibroblasts. Additionally, the present disclosure demonstrates one utility of the disclosed methods and cells: for modeling late-onset disorders such as Parkinson's disease and teaches the establishment of similar models for other diseases. The hypomethylated cell can be, or can be derived from, an iPSC or can be or can be derived from another type of stem cell, such as embryonic stem cells, skin stem cells from adult individuals, mesenchymal stem cells, hematopoietic stem cells and the like. Indeed, hypomethylation can be used to induce aging in any type of somatic cell, such as a neuron, regardless of provenance. However, it is difficult to obtain neurons from healthy donors, so a combination of stem cell differentiation and hypomethylation is a preferred method to obtain neurons expressing an “old” chronological marker signature.

Table 2 presents a set of age-associated markers that are found in primary fibroblasts derived from aging donors, which markers are lost during the reprogramming of a fibroblast to an iPSC and that are not produced upon differentiation of such an iPSC to a differentiated cell, such as a fibroblast-like cell or an mDA neuron. That is, reprogramming/differentiation generates cells having “young” phenotype (which would be age-inappropriate for studying late-onset diseases) regardless of the age of the somatic cell donor. Age-associated markers can, however, be reestablished upon reducing the level of genomic nucleic acid methylation, thereby giving rise to an “old” or mature iPSC-derived cell that would be age-appropriate for studying mature cells or late-onset disease or, in the case of mature cells, for use in therapy.

For example, iPSCs derived from a Parkinson's disease (PD) patient and an apparently healthy donor appear to be phenotypically identical despite their genotypic differences. Upon differentiation into mDA neurons only minor differences were observed between a PD cell versus a control cell (no/mild disease signature). In certain embodiments, hypomethylation triggers an mDA aging-like signature in an iPSC-derived mDA neuronal cell and also reveals multiple disease-associated (PD-associated) phenotypes that have interactions between genotype and phenotype in PD iPSC-derived mDA neurons (i.e., enhanced disease signature).

TABLE 2
Representative Phenotypes and Associated Markers

| | |
|---|---|
| Phenotypes | Method of detection |

Fibroblast aging signature

| | |
|---|---|
| Nuclear folding and | Lamin A/C |
| blebbing | |
| Loss of nuclear organization | LAP2α |
| proteins | |
| Loss of heterochromatin | H3K9me3, HP1γ |
| Accumulation of DNA | γH2AX |
| damage | |
| Increased mitochondrial | MitoSOX |
| ROS generation | |
| Telomere shortening | Telomeric repeats Q-FISH |
| | probe |
| Upregulation of senescence | SA-β-Gal |
| markers | |

mDA neuron aging signature

| | |
|---|---|
| Enhanced nuclear folding | Lamin A/C |
| and blebbing | |
| Accumulation of DNA | γH2AX |
| damage | |
| Increased mitochondrial | MitoSOX |
| ROS generation | |
| Dendrite shortening | MAP2ab |
| Neurodegeneration gene | RNA-seq |
| expression signature | |
| Hyperactivation of p-AKT | p-AKT, p-4EBP1, p-ULK1 |
| Mild decrease of TH+ | TH in vivo |
| neurons | |
| Accumulation of | Electron microscopy in |
| neuromelanin | vivo |

PD disease signature

| | |
|---|---|
| Enhanced susceptibility to | Cleaved caspase-3 |
| cell death activation | |
| Accelerated dendrite | MAP2ab |
| shortening | |
| Loss of p-AKT | p-AKT, p-4EBP1, p-ULK1 |
| Pronounced/progressive | TH in vivo |
| loss of TH+ neurons | |
| Enlarged mitochondria | Electron microscopy in |
| | vivo - PINK1 only |
| Multilamellar inclusions | Electron microscopy in |
| | vivo - Parkin only |

Markers that predict a somatic cell donor's age, which can be used to monitor cellular age during reprogramming, differentiation, and induced aging, include telomere length, which is shortened as the cell ages and which is restored by reprogramming and the resulting production of functional telomerase. Agarwal et al., Nature 464:292-296 (2010) and Marion et al., Cell Stem Cell 141-154 (2009)). Similarly, iPSC induction rejuvenates the mitochondria of aged cells. Prigione et al., Stem Cells 721-733 (2010) and Suhr et al., PloS One 5:e14095 (2010). Those studies were limited, however, to a comparison of individual phenotypes between cell types that are highly distinct (fibroblasts versus iPSCs). In contrast, the present disclosure provides a range of age-related markers, which markers correlate with cellular age and corresponding cell fates (donor fibroblast versus iPSC-derived fibroblast).

Additional suitable markers include, but are not limited to, methylation levels at particular CpG sites, which are predictive of donor age across multiple tissues (Horvath, S. Genome Biol 14, R115 (2013); Hannum et al., Mol. Cell. 49:359-367 (2013) and Koch and Wagner, Aging 3:1018-1027 (2011)) and methylation patterns that reflect epigenetic memory in iPSCs of donor cell fate (Kim et al., Nature 467:285-290 (2010) and Polo et al., Nat Biotechnol 28:848-855 (2010)).

The present disclosure describes methods of inducing hypomethylation in cells to induce cell type-specific responses in different cell lineages. (Table 2). Moreover, in certain embodiments, the present application describes methods for inducing hypomethylation for reestablishing age in cells, such as fibroblasts, and to phenocopy certain aspects of normal neuron aging, such as the presence of neuromelanin in grafted mDA neurons, global transcriptional changes in mDA neurons, and in vitro dendrite degeneration phenotype. In certain embodiments, the degenerative neuronal response occurs after a fiber network has been established, and is distinct from the reduced primary fiber outgrowth that may also reflect a “neurodegeneration” phenotype. Sánchez-Danes et al., EMBO Molecular Medicine 4:380-395 (2012).

The present disclosure can also be applied to induce aging of a variety of cell lineages. These cells include major cell types found in a variety tissues and organs, including, but not limited to, brain, heart, liver, kidney, spleen, muscle, skin, lung, blood, artery, eye, bone marrow, and lymphatic system. For example, Table 3 lists additional cell types and their aging markers that can benefit from hypomethylation-induced aging or maturation in vitro (See e.g., A. Sheydina et al., Clinical Science (2011) 121, (315-329); U. Gunasekaran and M. Gannon, 2011, Aging, 3(6): 565-575). In addition, although iPSC-derived cells have been used to study neurodegenerative diseases as summarized in Table 4, including ALS, Parkinson's disease and Alzheimer's disease, these iPSC-derived neurons are not age-modified and thus may not adequately represent neurons in these late-onset diseases.

TABLE 3
Additional Cell Aging Phenotypes and Associated Markers

| | |
|---|---|
| Cell Phenotypes | Markers and method of detection |

Cardiomyocytes

| | |
|---|---|
| Reduced contractile and | MHC, SERCA2, NCX1, mitochondrial proteins and |
| luistropic function | heteroplasmy, and Cx43 |
| Increased cell | ANP, BNP, ERK1/2, NFAT, calcineurin and S6 |
| diameter/hypertrophy | kinases |
| Fibrosis and apoptosis | TERT, IGF-1, PI3K, ET-1, SIRT1 SIRT7, caspases, |
| | AIF and survivin |
| Reduced proliferation | Cyclin D1, cyclin D2, cyclin D3, pRb, p130 and |
| | CDK2 |
| Organization of sarcomeric | IHC, EM |
| proteins, calcium handling, | |
| and electrophysiology | |
| properties, poor graft-host | |
| integration and arrhythmias | |

Pancreatic β cells

| | |
|---|---|
| Decreased insulin secretion | ATP production, glucose oxidation, K_{ATP}-channel, |
| | Foxm1, Pdx1, |
| Loss of proliferation capacity | MTS assay, D cyclins, p16Ink4a, Cdk4/6 |
| Amylin aggregation | IAPP/amylin |
| Glucose responsiveness | Glucose tolerance and insulin response assays |

Kidney cell

| | |
|---|---|
| Tubular atrophy, fibrosis, | IHC, Electron microscopy |
| glomerulosclerosis | |
| Extracellular matrix and | MMP20, IGF1R, FAM83F, MMP25, ADCY1 |
| complement activation genes | |

Osteoblasts

| | |
|---|---|
| Terminal differentiation | Col1A1, osteocalcin, osteonectin, osteopontin, ALP |
| Mineralization | Calcium deposit, ALP |

Osteoclasts

| | |
|---|---|
| Terminal differentiation and | Cathepsin K, MMP9, RANKL |
| polarization | |

Hepatocytes

| | |
|---|---|
| Increase in nuclei size and | IHC, Electron microscopy |
| polyploidy, and mitochondrial | |
| volume | |
| Lipofuscin deposition | Lipofuscin, decline in intracellular proteinolysis. |

Dopamine neurons

| | |
|---|---|
| Apoptosis | DAT, pacemaker activity, neuromelanin |

Hematopoietic stem cells

| | |
|---|---|
| Differentiation marker | Notch signaling |

TABLE 4
iPSC-based disease models

| | Disease phenotypes | |
|---|---|---|
| | not observed that | |
| Disease of iPSC- | could benefit from | |
| based model | hypomethylation | Reference |
| ALS | Cytoplasmic | Bilican et al., 2012 Proc Natl Acad Sci U |
| | aggregates, decreased | S A. 2012 Apr. 10; 109(15): 5803-8; |
| | cell survival, altered | Burkhardt et al., 2013 Mol Cell |
| | neurite development | Neurosci. 2013 September; 56: 355-64; Egawa et |
| | | al., |
| | | 2012 Sci Transl Med. 2012 Aug. |
| | | 1; 4(145): 145ra104 |
| Alzheimer's disease | Decreased cell | Israel et al., 2012 Nature. 2012 Jan. |
| | survival | 25; 482(7384): 216-20; Koch et al., 2012 |
| | | Am J Pathol. 2012 June; 180(6): 2404-16 |
| Parkinson's disease | Loss of TH, | Cooper et al., 2012 Prog Brain |
| | decreased cell | Res. 2012; 200: 265-76; Nguyen et al., |
| | survival | 2011 Cell Stem Cell. 2011 Mar. |
| | | 4; 8(3): 267-80; Seibler et al., 2011 J |
| | | Neurosci. 2011 Apr. 20; 31(16): 5970-6.; |
| | | Chung et al., 2013 Am J Ophthalmol. |
| | | 2014 February; 157(2): 464-469 |

As disclosed herein, reducing the level of genomic nucleic acid methylation can mimic normal aging, which is the basis for the present methods for producing cells having an aged-like state, which cells are suitable for modeling late-onset diseases such as PD.

In certain embodiments, the methods of the present application comprise contacting a cell with an agent that inhibits or reduces nucleic acid methylation in an amount and for a period of time sufficient to reduce or inhibit the level of nucleic acid methylation in the cell.

As disclosed herein, the present application provides for methods of reducing the level of nucleic acid methylation in a cell in an amount that will be sufficient to induce accelerated aging and/or maturation of the cell. In certain embodiments, the level of methylation is reduced to a level between about 0.1 and 95%, or any values in between, for example, between about 1 and 95%, or between about 5 and 95%, or between about 10 and 95%, or between about 15 and 95%, or between about 20 and 95%, or between about 25 and 95%, or between about 30 and 95%, or between about 35 and 95%, or between about 40 and 95%, or between about 45 and 95%, or between about 50 and 95%, or between about 55 and 95%, or between about 60 and 95%, or between about 65 and 95%, or between about 70 and 95%, or between about 75 and 95%, or between about 80 and 95%, or between about 85 and 95%, or between about 90 and 95%, or between about 5 and 95%, or between about 5 and 90%, or between about 5 and 85%, or between about 5 and 80%, or between about 5 and 75%, or between about 5 and 70%, or between about 5 and 65%, or between about 5 and 60%, or between about 5 and 55%, or between about 5 and 50%, or between about 5 and 45%, or between about 5 and 40%, or between about 5 and 35%, or between about 5 and 30%, or between about 5 and 25%, or between about 5 and 20%, or between about 5 and 15%, or between about 5 and 10%, of a cell whose level of methylation is not reduced according to the methods described herein, for example, a young cell.

In certain embodiments, the level of methylation is reduced to a level between about 1 and 70%, or between about 5 and 60%, or between about 10 and 50%, or between about 15 and 40%, or between about 20 and 30% of a cell whose level of methylation is not reduced according to the methods described herein, for example, a young cell.

As disclosed herein, the present application provides for methods of reducing the level of nucleic acid methylation in a cell in an amount that will be sufficient to induce accelerated aging and/or maturation of the cell. In certain embodiments, the level of methylation is reduced by between about 0.1 and 90%, or between about 1 and 70%, or between about 5 and 60%, or between about 10 and 50%, or between about 15 and 40%, or between about 20 and 30% from the level of nucleic acid methylation in a cell whose level of methylation is not reduced according to the methods described herein, for example, a young cell.

In certain embodiments, the agent that inhibits or reduces nucleic acid methylation comprises a nucleoside analog of cytidine, for example, zebularine (also known as 1-(β-D-Ribofuranosyl)-2(1H)-pyrimidinone or Pyrimidin-2-one β-D-ribofuranoside). In certain embodiments the zebularine is administered at a concentration of between about 5 and 70 μM, or any values in between, for example between about 10 and 70 μM, or between about 15 and 70 μM, or between about 20 and 70 μM, or between about 30 and 70 μM, or between about 40 and 70 μM, or between about 50 and 70 μM, or between about 60 and 70 μM, or between about 5 and 60 μM, or between about 5 and 50 μM, or between about 5 and 40 μM, or between about 5 and 30 μM, or between about 5 and 20 μM, or between about 5 and 15 μM, or between about 5 and 10 μM.

In certain embodiments, the agent that inhibits or reduces nucleic acid methylation comprises 5-aza-2-deoxycytidine (5-aza-dC; Decitabine) and/or homocysteine and/or the homocysteine metabolite S-adenosyl-1-homocysteine (SAH). In certain embodiments the Decitabine is administered at a concentration of between about 0.05 and 5 μM, or any values in between, for example, between about 0.1 and 5 μM, or between about 0.5 and 5 μM, or between about 1 and 5 μM, or between 0.05 and 1 μM, or between about 0.05 and 0.5 μM, or between about 0.05 and 0.1 μM.

In certain embodiments, the agent that inhibits or reduces nucleic acid methylation comprises 4-Chloro-N-(4-hydroxy-1-naphthalenyl)-3-nitro-benzenesulfonamide (SW155246). In certain embodiments the SW155246 is administered at a concentration of between about 0.05 and 10 μM, or any values in between, for example between about 0.1 and 10 μM, or between about 1 and 10 μM, or between about 5 and 10 μM, or between about 0.05 and 5 μM, or between about 0.05 and 1 μM, or between about 0.05 and 0.1 μM.

In certain embodiments, the agent that inhibits or reduces nucleic acid methylation comprises (3S,3'S,5aR,5aR,10bR,10′bR,11aS,11′aS)-2,2′,3,3′,5a,5′a,6,6′-octahydro-3,3′-bis(hydroxymethyl)-2,2′-dimethyl-[10b,10′b(11H,11′H)-bi3,11a-epidithio-11aH-pyrazino[1′,2′:1,5]pyrrolo[2,3-b]indole]-1,1′,4,4′-tetrone, (Chaetocin). In certain embodiments the Chaetocin is administered at a concentration of between about 0.0001 and 1 μM, or any values in between, for example, between about between about 0.001 and 1 μM, or between about 0.01 and 1 μM, or between about 0.1 and 1 μM, or between about 0.0001 and 0.1 μM, or between about 0.0001 and 0.01 μM, or between about 0.0001 and 0.001 μM.

In certain embodiments, the agent comprises an inhibitor of a DNA methyltransferase (DNMT) and/or an inhibitor of histone methyltransferase (HMT), for example, an antibody or fragment thereof that binds to a DNMT and/or an HMT, or an antisense or siRNA molecule that reduced or inhibits expression of a DNMT and/or HMT enzyme. In certain embodiments the DNMT enzyme comprises DNMT1, DNMT3A, DNMT3B, and/or DNMT3L.

In certain embodiments, the agent comprises an inhibitor of a histone methyltransferase, for example, SUV3/9, for example, an antibody or fragment thereof that binds to a histone methyltransferase, or an antisense or siRNA molecule that reduced or inhibits expression of a histone methyltransferase.

In certain embodiments, the agent comprises an inhibitor of a methyl-CpG-binding protein (MeCP2) and/or an inhibitor of a PHD and RING finger domains 1 protein (UHRF1), for example, an antibody or fragment thereof that binds to a MeCP2 and/or UHRF1 protein, or an antisense or siRNA molecule that reduced or inhibits expression of a MeCP2 and/or UHRF1 protein.

In certain embodiments, the agents described herein that reduce nucleic acid methylation are contacted to a cell for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days. In certain embodiments, the agents are contacted to the cells for up to about 1 day, up to abut 2 days, up to about 3 days, up to about 4 days, up to about 5 days, up to about 6 days, up to about 7 days, up to about 8 days, up to about 9 days, up to about 10 days, up to about 11 days, up to about 12 days, up to about 13 days, up to about 14 days, up to about 15 days, up to about 16 days, up to about 17 days, up to about 18 days, up to about 19 days, or up to about 20 days.

In certain embodiments, the level of nucleic acid methylation is reduced by a mutation, for example, a mutation introduced into the nucleic acid of a cell through any methods known in the art, such as site-directed mutagenesis, wherein the mutation is in a nucleic acid encoding a DNA methyltransferase (DNMT) and/or a histone methyltransferase (HMT) and/or a methyl-CpG-binding protein (MeCP2) and/or a PHD and RING finger domains 1 protein (UHRF1), for example, a hypomorphic mutation, such as a hypomorphic mutation in DNMT1.

In certain embodiments, the reduction of nucleic acid methylation comprises a reduction in the level and/or rate of methylation at one or more CpG methylation sites.

In certain embodiments, the reduction in the level of nucleic acid methylation comprises a reduction of methylation at non-coding regions of genomic nucleic acid repetitive elements, for example, LINE1 (L1) elements, LTR elements, and/or Endogenous Retroviruses (ERV) elements. In certain embodiments, the amount of repetitive elements in the aged cell that is hypomethylated compared to a young cell is between about 10 and 90%, and any values in between, for example, between about 15 and 90%, or between about 20 and 90%, or between about 25 and 90%, or between about 30 and 90%, or between about 35 and 90%, or between about 40 and 90%, or between about 45 and 90%, or between about 50 and 90%, or between about 55 and 90%, or between about 60 and 90%, or between about 65 and 90%, or between about 70 and 90%, or between about 75 and 90%, or between about 80 and 90%, or between about 85 and 90%, or between about, between about or between about 10 and 85%, or between about 10 and 80%, or between about 10 and 75%, or between about 10 and 70%, or between about 10 and 65%, or between about 10 and 60%, or between about 10 and 55%, or between about 10 and 50%, or between about 10 and 45%, or between about 10 and 40%, or between about 10 and 35%, or between about 10 and 30%, or between about 10 and 25%, or between about 10 and 20%, or between about 10 and 15%, of the cell's repetitive elements.

In certain embodiments, the reduction in nucleic acid methylation achieved by the methods of the present application reduces epigenetic silencing of DNA transcription, wherein such a reduction of epigenetic silencing comprises a reduction in the levels of repressive histone marks, for example, H3K9me3 and/or H3K27me3.

In certain embodiments, the reduction in the level of nucleic acid methylation comprises a reduction in the levels of histone protein H1.

In certain embodiments, the reduction in the level of nucleic acid methylation comprises a reduction in the levels of heterochromatin marker HP1α.

In certain embodiments, the reduction in the level of nucleic acid methylation comprises a reduction in the levels of nuclear morphology marker LaminB1.

In certain embodiments, the reduction in the level of nucleic acid methylation comprises an increase in the levels of yH2Ax, a marker of DNA damage.

In certain embodiments, the reduction in nucleic acid methylation achieved by the methods of the present application increases the transcription expression of repetitive elements, for example, LINE1 (L1) elements, LTR elements, and/or Endogenous Retroviruses (ERV) elements, in the aged cells compared to young cells. In certain embodiments, the repetitive elements that have an increased expression in the aged cells comprise elements are low abundance elements having Fragments Per Kilobase of transcript per Million mapped reads (FPKM) of between about 10 and 1,000, between about 20 and 500, between about 30 and about 50, between about 40 and about 200, between about 50 and about 150, or between about 60 and about 100 FPKM.

In certain embodiments, the methods of the present application for reducing nucleic acid methylation in a cell comprises increasing the level of 5-hydroxy-methyl-cytosine (5hmC) nucleic acid modifications in the cell.

In certain embodiments, the methods of the present application comprise increasing the level or activity of ten-eleven translocation (TET) proteins in the cell, for example, but not limited to, human ten-eleven translocation 1 (TET1).

### 5.2.1 Hypomethylation-Mediated Age Acceleration of iPSC-Derived Somatic Cells

In certain embodiments, the present disclosure provides a set of chronological marker signatures that correlate with donor age, for example the age of a fibroblast donor, which marker signatures include, but are not limited to, markers of nuclear morphology and expression of nuclear organization proteins as well as markers of heterochromatin, DNA damage, and reactive oxygen species. These age-associated chronological marker signatures in “old” fibroblasts are lost during reprogramming and are not reacquired during subsequent differentiation, supporting the hypothesis that iPSC-derived cells do not maintain age memory. Tissue-specific age-associated marker signatures can be induced in both iPSC-derived fibroblasts and mDA neurons following short-term genomic nucleic acid methylation reduction exposure. The ability to rapidly induce chronological marker signatures that are associated with cellular age is employed in methods disclosed herein for modeling Parkinson's disease in vitro and following transplantation of iPSC-derived mDA neurons in vivo.

As disclosed herein, several age- and PD-related phenotypes, which are not observed using current iPSC technologies, as provided by cells of the present disclosure, include, but are not limited to, dendrite degeneration, formation of age-associated neuromelanin, AKT deregulation, selective reduction in the number of TH+ neurons, ultrastructural evidence of mitochondrial swelling and inclusion bodies, and the like. Induced aging provides model systems for iPSC studies and that may be adapted to other cell types and disease pathologies to address the contribution of genetic and age-associated susceptibility in late-onset disorders.

Thus, the present disclosure provides chronological marker signatures including, but not limited to, global genetic and epigenetic signatures, as models for primary somatic cells, primary fibroblasts, iPSC-derived somatic cells, iPSC-derived fibroblasts and/or iPSC-derived neurons, such as iPSC-derived midbrain dopamine neurons. In certain aspects, the chronological marker signatures reflect cellular behaviors capable of identifying genome-wide genetic and epigenetic profiles as precise signatures of cellular age. Cellular age can, for example, be determined by interactions between age-related markers with genetic and/or via epigenetic profiles.

Fibroblasts and neurons are known to have specific biomarkers based upon the particular age of the donor individual. The present disclosure demonstrates that age-related markers in primary fibroblasts (young or old) can be “re-set” during iPSC induction to an embryonic-stage marker signature. Subsequently, an embryonic-stage marker signature is largely unchanged upon differentiation. The immature/embryonic/young age-related marker signatures can then be converted to an old age-related marker signature (or to a mature age marker signature) upon reducing the level of genomic nucleic acid methylation. Such age-related markers include, but not limited to, those listed in Table 2 and Table 3.

### 5.2.2 Methods for Inducing Aging in iPSC-Derived Neuronal Cells

Within certain embodiments, the present disclosure provides methods for directing in vitro neuronal aging by reducing genomic nucleic acid methylation to establish a disease model of a late-onset neurodegenerative disorder. Without intending to be bound by theory, it is believed that markers associated with a donor's age and/or disease are reset during iPSC-based reprogramming and are not re-established following subsequent differentiation into iPSC-derived lineages. The present disclosure, therefore, provides methods for differentiating iPSC-derived lineages and reestablishing one or more age-associated and/or disease-associated markers, the presence or absence of which markers may comprise one or more age-associated and/or disease-associated marker signatures and/or cell behaviors. In certain aspects of these embodiments, iPSC differentiation is initiated by one or more compounds including, but not limited to, a Wnt inhibitor and/or one or more SMAD inhibitor. Methods of differentiating iPSCs are described by International Application Nos. PCT/US10/024487, filed Feb. 17, 2010; PCT/US11/037179, filed May 19, 2011; PCT/US12/063339, filed Nov. 2, 2012; PCT/US14/035760, filed Apr. 28, 2014; PCT/US14/066952, filed Nov. 21, 2014; PCT/US14/034435, filed Apr. 16, 2014; and U.S. Provisional Application Nos. 62/169,444, filed Jun. 1, 2015; and 62/169,379, filed Jun. 1, 2015; each of which is incorporated by reference in its entirety.

The advent of iPSC technology has the potential to accelerate the development of therapies for a broad range of genetic disorders and provides a cell culture platform on which routine studies of disease processes may be replicated. The iPSC approach can also yield mechanistic insights into a disease process and therefore identify target sites for future drug development.

Disclosed are methods for introducing an age component into iPSC-based models of late-onset disorders. As described herein, the reprogramming of established somatic cell cultures produces immature induced pluripotent stem cell-derived cell types, which do not exhibit late-onset disorder and/or disease phenotypes as develop in an affected aged individual. Thus in one embodiment, the present disclosure provides methods for introducing “age” and/or “maturation” into iPSC-derived cell types by reducing the level of genomic nucleic acid methylation. Examples of various cell types and the markers for aging include, but not limited to, those listed in Table 2 and Table 3.

In one aspect of these methods, an iPSC-derived somatic cell exhibits one or more markers of a late-onset disease and/or disorder phenotype. In related aspects of these methods, the more permissive state comprises one or more cellular responses that are closely aligned with those observed in the in vivo aged PD brain. As disclosed herein, one or more chronological marker, which comprises one or more chronological marker signature, can be monitored, reprogramed, and/or induced in iPSC cell cultures. Inducing chronological marker signatures in iPSC-derived cell culture models improves late-onset human disease modeling and therapeutic target discovery and, more generally, addresses fundamental questions related to human disease and age.

The methods disclosed here employ iPSC technology to reset and re-establish age-related markers in neuronal disease cell culture models. Certain epigenetic features, such as residual DNA methylation of the donor cell type, may be retained, at least transiently, following iPSC derivation (Kim et al., Nat Biotechnol 29:1117-1119 (2011); Kim et al., Nature 467:285-290 (2010); and Polo et al., Nat Biotechnol 28:848-855 (2010)). The present disclosure provides data, which demonstrate that the reprogramming of chronological markers in an aged primary cell, such as, for example, a fibroblast, are not reacquired upon conversion to an iPSC and subsequent differentiation of a reprogrammed iPSC to a somatic cell, such as a fibroblast cell and/or a neuronal cell.

Thus, the present disclosure provides methods for comparing chronological marker signatures and/or functional features in different cell types. For example, both proliferating cells (e.g., astrocytes) and post-mitotic cells (e.g., neurons) within the central nervous system may be produced and aged by the methods disclosed herein and used as model systems for studying relationships between cell proliferation and maturation and cell-type specific aging signatures. Examples of various cell types and the markers for aging include, but not limited to, those listed in Table 2 and Table 3.

In certain embodiments, the level of genomic nucleic acid methylation is reduced in a cell by contacting the cell with an inhibitor of methylation, for example, a nucleoside analog of cytidine, for example, zebularine (also known as 1-(β-D-Ribofuranosyl)-2(1H)-pyrimidinone or Pyrimidin-2-one β-D-ribofuranoside).

In certain embodiments, the agent comprises an inhibitor of a DNA methyltransferase (DNMT), for example, an antibody or fragment thereof that binds to a DNMT, or an antisense or siRNA molecule that reduced or inhibits expression of a DNMT enzyme.

### 5.2.3 Global Transcriptome and Methylation

The present disclosure provides methods for evaluating global transcriptomes by sequencing of mRNA by, for example, RNA-Seq. The present disclosure also provides for methods of measuring global DNA methylation by measuring 5-methylcytosine (5-mC) and/or DNA methylation signatures, for example, using ERRBS (enhanced reduced-representation bisulfite sequencing). For example, one component of cell development and reprogramming comprise epigenomic modifications of DNA methylation and histone markers. Both 5-methylcytosine (5-mC) and 5-hydroxymethylcytosine (5-hmC) are drastically modified during neuronal differentiation and under neurodegenerative conditions (Szulwach et al., Nat Neurosci 14:1607-1616 (2011) and Trier & Jin, DNA Cell Biol 31(Suppl 1):S42-48 (2012), respectively). The functional role of these epigenetic markers has not, however, been fully explored in the context of differentiated iPSC and in vitro age modeling. Thus, the reprogramming/differentiation/aging paradigm disclosed here for modeling late-onset of neurodegenerative disease provides a platform on which to explore these epigenetic changes and their impact on gene expression.

The methods described herein provide for determining genome-wide 5-mC and 5-hmC profiles in age-associated primary fibroblasts and their iPSCs. For example, iPSCs are significantly more methylated in 5-mC markers than their primary fibroblasts (Wu & Zhang, Cell Cycle 10:2428-2436 (2011)).

In one embodiment, the present disclosure provides methods for integrating DNA methylation data and transcriptomics data with phenotypic age-related marker signature data to provide a more precise cellular description of age. In one embodiment, a more accurate characterization of age (e.g., by measuring expression of several aging markers, e.g., those listed in Table 2 and Table 3) improves modeling of late-onset diseases using somatic cell cultures, such as iPSC-derived cell cultures.

Data integration can be performed by a number of computational analyses to identify the functional impact of the (epi)genetic changes on aging. For example an integrated analysis of DNA methylation and gene expression may be performed to identify dysregulated pathways and “driving” events that distinguish “old” fibroblasts. These analyses involve identifying the functional relationship between the epigenetic and transcriptional changes present in aged fibroblasts and hypomethylation-induced iPSC-fibroblasts.

One approach is to identify direct regulation of gene expression by methylation status. Differentially methylated regions, from either 5-mC or 5-hmC profiling, may be associated with proximal genes, which are most likely regulated by the DNA methylation. Next, gene expression changes may be identified that are most correlated with these methylation changes.

Another approach is to focus on identifying common pathways that are mutually regulated by both epigenetic and transcriptional changes. Functional enrichment analyses may be performed on genes identified by aberrant methylation status and/or genes that are differentially expressed (Subramanian et al., Proc Natl Acad Sci US A 102:15545-15550 (2005)).

Network connectivity of these gene sets can be investigated using tools including, but not limited to, SPIA43, NetBox44, and Enrichment Map, all of which take into consideration the interactions between the genes to identify functional “modules”—a group of interconnected genes that participate in a specific cellular function or pathway and are co-regulated (Merico et al., PLoS One 5:e13984 (2010)). Hence, pathways that are represented with high frequency in both methylation and gene expression datasets are likely to be functionally relevant for the aging process.

A correlation of gene expression signatures and perturbed functional pathways with age-related marker signatures can determine genetic processes that drive cellular aging. An example involves modeling quantitative readouts from age-related biomarker signatures (e.g., heterochromatin state, DNA damage and nuclear morphology) as a function of the genetic alteration such as, differential expression and/or differential methylation. Regression models (such as ridge regression, lasso regression, partial least squares (PLS) regression, or support vector regression) can correlate differentially methylated regions with heterochromatin changes measured quantitatively by H3K9me3 marker. Similarly, differentially methylated and expressed genes can be used in supervised classification schemes, using algorithms such as naïve Bayes classifiers, logistic regression and support vector machines, to distinguish DNA damage response from damaged mitochondria function.

These contemplated computational models also may provide a minimal set of genomic features that are most predictive of the aging phenotype. Various feature selection approaches, both in the regression and classification schemes, can be used to identify the genes and epigenetic modifications that are most predictive of the age biomarker assays. For example, qPCR may be used to identify a subset to be used for validation purposes. Alternatively, RNAi experiments can be used to test for functional relationships (Lipchina et al. Genes Dev 25:2173-2186 (2011)).

### 5.2.4 Functional Characteristics of Cellular Aging

In some embodiments, the present disclosure provides methods for testing a relationship between induced aging and chronological aging. In certain aspects of those embodiments, the induced aging process is reversible. In other aspects, the induced and chronological aging models comprise novel marker sets that are relevant to studying age in human brains.

### 5.2.5 Age-Related Marker Sensitivity

Age-related marker sensitivity may be tested in vitro using chronological and hypomethylation-induced cellular aging. For example, changes in age-related markers may occur suddenly once a donor has reached a certain age (e.g., >70 years of age) or there may be a gradual increase in the expression of age associated markers as one gets progressively older.

Somatic cells, for example, primary fibroblasts of three different age groups: (i) 0-15 years, (ii) 30-50 years, (iii) 70-90 years can be obtained from healthy donor individuals. A determination of the established age-related marker signatures (e.g., nuclear lamina structure, heterochromatin, DNA damage and mitochondrial damage) as well as newly discovered age-related marker signatures and/or genetic markers can determine relationships between marker expression and fibroblast donor individual age. For example, age-related marker signature expression can be determined in three independent replicates for each fibroblast line maintained at identical passage numbers and for at least three fibroblast lines for each age group. These time-course data are compared to iPSC derived fibroblast lines from 82 year old donor individuals and iPSC-derived fibroblasts lines from 11 year old donor individuals wherein the level of genomic nucleic acid methylation is reduced.

Analysis of these data may determine the sensitivity of age-related assays and the relationship among various age markers. Those data may yield information about existing hierarchies within and among age-related phenotypes. Second, these data may be used to pinpoint the “age equivalent” for a given phenotype in iPSC-derived fibroblasts treated with hypomethylation versus primary fibroblasts of various donor ages and assess whether the temporal changes following hypomethylation match the chronological changes observed in primary fibroblasts from donor individuals of increasing age.

### 5.2.6 Fibroblast Reversibility

In some embodiments, the present disclosure provides methods for reducing or increasing the level of genomic nucleic acid methylation in a fibroblast. In related aspects, the methods further comprise monitoring the fibroblast for a sequence in age-related marker signature phenotype alterations.

### 5.2.7 Neuronal Reversibility

In certain embodiments, the present disclosure provides methods for reducing or increasing the level of genomic nucleic acid methylation in a midbrain dopamine neuronal culture. In other aspects, the methods further comprise monitoring the neurons for a sequence in age-related marker signature phenotype alterations.

### 5.3 Parkinson's Disease Modeling

Parkinson's disease has a prevalence of approximately 0.5-1.0×10⁶ patients affected in the United States. Symptoms include, but are not limited to, rigor, tremor, bradykinesia (slow movement) and/or poor balance/walking. Clinical pathology diagnoses PD primarily due to a loss of midbrain dopamine neurons. The etiology of PD is mostly unknown and sporadic, but multiple genes are involved in familial forms of PD.

In one embodiment, the present disclosure provides methods comprising inducing cellular aging to create late-onset neurodegenerative disease cells, which can, for example, be employed as PD model systems. In certain aspects, the cells are induced pluripotent stem cells.

Induced cellular aging provides a system to model age-related aspects of late-onset neurodegenerative diseases. Such a system can be used to directly test an interaction between genetic susceptibility and age-related vulnerability on disease phenotype.

The present disclosure also provides methods for inducing cellular age in iPSC-derived mDA neurons. In certain aspects, these methods may be employed for modeling of age-dependent effects in Parkinson's disease (PD). The data presented herein address the following issues, for example: (i) using directed differentiation techniques for the generation of authentic mDA neurons; (ii) establishing a broad range of genetic PD-iPSC lines; (iii) validating age-related marker signature phenotypes in iPSC-mDA neurons; (iv) demonstrating an interaction between age phenotypes and disease phenotypes; and (v) establishing gene-edited PD-iPSC lines. These gene-edited lines may contribute to the understanding of genetic susceptibility versus age-induced vulnerability.

The present disclosure also provides cells comprising at least one PD-iPS cell. In one aspect, the PD-iPS cell originates from PD patient skin fibroblasts. In other aspects, the fibroblasts that give rise to PD-iPS cells comprise at least one mutation selected from the group comprising Parkin, PINK1, LRRK2, α-synuclein, and glucocerebrosidase (GBA) (Kitada et al., Nature 392:605-608 (1998); Valente et al. Science 304:1158-1160 (2004); Zimprich et al., Neuron 44, 601-607 (2004); Polymeropoulos et al., Science 276:2045-2047 (1997); Toft et al., Neurology 66:415-417 (2006)). They thus express a disease phenotype.

In certain embodiments, the present application provides for methods of inducing hypomethylation to provide an accelerated dendrite degenerative and/or shortening phenotype in mDA neurons from iPSC-derived midbrain dopamine cell cultures from either PINK1-mutant or Parkin-mutant Parkinson's individuals.

### 5.4 Methods of Treatment

Cells may be isolated from healthy subjects, at risk subjects, and diseased subjects for use in generating undifferentiated iPS cells according to methodology presented herein or as otherwise available in the art. Primary somatic cells used for reprogramming may be isolated from a variety of bodily locations, such as circulating cells and/or cells in tissues of patients/subjects, including but not limited to fibroblasts, skin fibroblasts, white blood cells, circulating white blood cells, mucosal cells, and keratinocytes without regard for the “age” of the cell or the “age” of the donor. In some aspects, primary somatic cells may be young cells expressing a “young” cell marker signature isolated from young donors, which cells may or may not be expressing a disease signature. In other aspects, primary somatic cells may be old cells expressing an “old” marker signature. In further aspects, primary somatic cells may be cells expressing a disease marker signature regardless of the chronological age of the donor. These primary cells can be reprogrammed in culture to give rise to iPSC using any method for generating iPSC from somatic cells. Such methods, other than described or referenced herein, are known in the art.

Generated iPSCs of any origin, including cells generated by methods described herein, may be used in differentiation protocols for producing differentiating and differentiated iPSC-derived cells that may find use in hypomethylation aging compositions and methods of the present disclosures. Differentiating and differentiated iPSC-derived cells include but are not limited to default and nondefault differentiation lineages, including partially differentiated (i.e., differentiating) cells, so long as they are capable of expressing genetic and cell marker signatures of their particular cell types, i.e., permissive cells. Examples of cell types which may find use in aging induction using hypomethylation methods of the present disclosure are iPSC-derived cells including but not limited to neurons (any subtype, such as motoneurons, cortical neurons, peripheral sensory neurons, mid-brain dopamine neurons etc.), cardiomyocytes, hematopoietic stem cells (HSCs), pancreatic beta cells, astrocytes, etc.

Thus, iPSC derived cells at certain stages will find use in hypomethylation treatment according to the present disclosure including, but not limited to, iPSC derived cells beginning to undergo differentiation, iPSC derived cells progressing towards committed cells types, iPSC derived cells progressing towards a mature cell type, etc. For example, an iPSC-derived midbrain dopamine neuron, or precursor thereof, (for example, from a healthy subject) can be aged according to the present disclosure and can be used in a cell based therapy for introducing into a PD patient. Accordingly, the present disclosure provides for pharmaceutical compositions comprising the aged cells described herein.

Nonlimiting examples of specific iPSC-derived cell types and associated disease(s) which can be used in conjunction with the methods of inducing aging described herein include iPSC derived-neurons for neurodegenerative diseases, iPSC-derived cardiomyocytes for degenerative cardiac diseases, iPSC-derived hematopoietic stem cells for leukemia and other white blood cell diseases and disorders and more generally hematopoietic diseases/disorders, iPSC-derived pancreatic beta cells for Type I diabetes, Type II diabetes and certain other types of insulin regulation disorders such as Type II diabetes, iPSC-derived motoneurons for ALS, iPSC-derived cortical neurons for Alzheimer's, iPSC-derived mDA neurons and iPSC-derived cortical neurons for corticobasal degeneration, iPSC-derived astrocytes for neurodegenerative disorders, iPSC derived cardiomyocytes for cardiac hypertrophy and fibrosis, and the like.

In some embodiments, iPSCs of the present disclosure are differentiated into somatic cell types that are immature or take a long time to mature (as assessed for example by protein expression in the cells, gene expression profiles, functional tests, etc.). Genomic nucleic acid methylation levels can be reduced in such immature cells to induce maturation in the cell population so these cells may be used in cell therapy. Examples of such immature iPSCs differentiated cells are iPSC-derived mDA neurons which lack pacemaker activity, expression of the dopamine transporter DAT, and neuromelanin and which require an additional 5 months of maturation in vivo to rescue Parkinsonian mice (Isacson et al., Trends Neurosci 20:477-482 (1997; Kriks et al., Nature 480:547-551 (2011). Furthermore, based on the BrainSpan: Atlas of the Developing Human Brain (http://www.brainspan.org), gene expression data from pluripotent stem cell-derived neural cells matches the transcriptome of first trimester embryos. Genomic nucleic acid methylation levels can be reduced in immature neurons for the purpose of accelerating their maturation as assessed for the markers listed above as characteristic of the desired mature neuronal subtype contemplated for use in cell therapy and/or drug development. In particular, cells provided by methods of the present disclosure may find use in drug screening, i.e., evaluation of compound candidates for aging control agents, agents for the treatment of specific diseases or disorders, such as those described herein, etc.

Other examples of using iPSC derived cells are hematopoietic stem cells (HSCs) derived from iPSCs which do not express signature markers of adult HSCs and could benefit from hypomethylation treatment to induce expression of an adult marker signature (including without limitation HoxB4, Tek (a/k/a Tie2) and HoxA9). For examples of other markers see, McKinney-Freeman et al., Cell Stem Cell 11:701-714 (2012), showing transcriptomes of developing HSC purified from mice.

As another example, cardiomyocytes derived from iPSCs are immature and will find use in methods of the present disclosure for identifying induction of maturation markers including but not limited to electrophysiological properties, such as higher sodium currents, reduced sensitivity to lidocaine, beating frequency, sensitivity to tetrodotoxin (TTX), and organizational patterns of sarcomeric proteins, such as actinin, etc.

Beta cells derived from iPSCs will find use in methods of the present disclosures and contemplated for use in cell therapy and/or drug development. In particular, reducing the level of genomic nucleic acid methylation in iPS derived beta cells can be used for inducing expression of a maturation marker Ucn3, along with a capability to induce insulin expression, and release of insulin in response to glucose not found in immature cells. For example, Blum-Melton et al, Nat Biotechnol 30:261-264 (2012) show where beta-cell maturation is defined by a decrease in GSIS sensitivity to low glucose levels and by an increase in expression of Ucn3 as shown by intracellular FACS analysis of insulin and Ucn3.

The presently disclosed aged (or rejuvinated) cells can be administered or provided systemically or directly to a subject for treating or preventing a disorder, for example, Parkinson's disease (PD) or Alzheimer's disease (AD). In certain embodiments, the presently disclosed cells are directly injected into an organ of interest (e.g., an organ affected by a neurological disorder, for example, the central nervous system (CNS)). The presently disclosed cells can be administered (injected) directly to a subject's CNS.

The presently disclosed cells can be administered in any physiologically acceptable vehicle. Pharmaceutical compositions comprising the presently disclosed cells and a pharmaceutically acceptable carrier are also provided. The presently disclosed cells and the pharmaceutical compositions comprising thereof can be administered via localized injection, orthotopic (OT) injection, systemic injection, intravenous injection, or parenteral administration. In certain embodiments, the presently disclosed cells are administered to a subject suffering from a neurological disorder (e.g., PD or AD) via orthotopic (OT) injection.

The presently disclosed cells and the pharmaceutical compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed cells, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently cells.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed cells. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the presently disclosed cells is the quantity of cells necessary to achieve an optimal effect. An optimal effect include, but are not limited to, repopulation of the CNS of a subject suffering from a neurological disorder (e.g., PD or AD), and/or improved function of the subject's CNS.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the neurological disorder (e.g., PD or AD), or otherwise reduce the pathological consequences of the neurological disorder (e.g., PD or AD). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the presently disclosed cells is an amount that is sufficient to repopulate the CNS of a subject suffering from a neurological disorder (e.g., PD or AD). In certain embodiments, an effective amount of the presently disclosed cells is an amount that is sufficient to improve the function of the CNS of a subject suffering from a neurological disorder (e.g., PD or AD), e.g., the improved function can be about 1%, about 5%, about 10%, about 20%, about 30%, about 40%, about 50%, about 60%, about 70%, about 80%, about 90%, about 95%, about 98%, about 99% or about 100% of the function of a normal person's CNS.

The quantity of cells to be administered will vary for the subject being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the presently disclosed cells are administered to a subject. In certain embodiments, from about 1×10⁵ to about 1×10⁷ of the presently disclosed s cells are administered to a subject suffering from a neurological disorder (e.g., PD or AD). In certain embodiments, about 2×10⁵ of the presently disclosed cells are administered to a subject suffering from a neurological disorder (e.g., PD or AD). In certain embodiments, from about 1×10⁶ to about 1×10⁷ the presently disclosed cells are administered to a subject suffering from a neurological disorder (e.g., PD or AD). In certain embodiments, from about 2×10⁶ to about 4×10⁶ the presently disclosed cells are administered to a subject suffering from a neurological disorder (e.g., PD or AD). The precise determination of what would be considered an effective dose may be based on factors individual to each subject, including their size, age, sex, weight, and condition of the particular subject. Dosages can be readily ascertained by those skilled in the art from this disclosure and the knowledge in the art.

In certain embodiments, the cells that are administered to a subject suffering from a neurological disorder (e.g., PD or AD) for treating a neurological disorder are a population of midbrain dopamine neurons that are differentiated and aged according to the methods described herein. In certain embodiments, the cells that are administered to a subject suffering from a neurological disorder (e.g, PD or AD) for treating a neurological disorder are a population of midbrain dopamine neuron precursors that are differentiated and aged according to the methods described herein.

### 5.5 Method of Screening Therapeutic Compounds

In some embodiments, aged iPSC-derived cell types obtained as described herein may find use in disease modeling and for identifying therapeutically relevant cell stages during development, such as identifying hypomethylation-aged cellular stages for use in testing new drug compounds for use as therapeutics and for actual use in treatment of patients. Thus in some embodiments, primary somatic cell donors for iPSC-derived cell types have a disease or a disease phenotype induced in iPSC-derived cell/tissue culture including but are not limited to actual or model neurodegenerative diseases, such as Parkinson's disease (PD), Alzheimer's disease (AD), tauopathies, i.e., a class of neurodegenerative diseases associated with the pathological aggregation of tau protein in the human brain, cardiomyocyte-related diseases (such as cardiac hypertrophy, cardiac fibrosis, channelopathies, for example pathologies of sodium channels, arrhythmias etc.), pancreatic diseases, hematopoietic diseases, metabolic diseases, cancer etc.

The presently disclosed aged cells can be used to model disorders, for example, a neurological disorder such as Parkinson's disease (PD) and Alzheimer's disease (AD), and serve as a platform to screen for candidate compounds that can overcome disease related defects. The capacity of a candidate compound to alleviate a disorder (e.g., PD or AD) can be determined by assaying the candidate compound's ability to rescue a physiological or cellular defect in a diseased cell, for example, an iPSC-derived midbrain dopamine neuron (mDA), or precursor thereof, wherein the iPSC is prepared from a somatic cell obtained from a PD patient.

The presently disclosed subject matter provides for methods of screening compounds suitable for treating a disorder (e.g., PD or AD) in vitro. In certain embodiments, the method comprises identifying a compound that is capable of rescuing at least one cellular disease phenotype, for example, as described by table 2 or 4.

In certain embodiments, the method comprises: (a) providing (i) a population of the presently disclosed aged cells (e.g., iPSC-derived PD neurons or progenitors thereof), and (ii) a test compound; (b) contacting the cells with the test compound; and (c) measuring the level or presence of one or more disease phenotype, for example, as described by table 2 or 4, wherein a test compound that reduces the level of presence of the one or more disease phenotype is selected as a candidate therapeutic compound.

### 5.6 Methods of Determining Molecular Age

The present disclosure provides methods for determining the molecular age of a cell, for example, an iPSC-derived cell, such as an iPSC-derived somatic cell (e.g., iPSC-derived fibroblasts and iPSC-derived neurons), wherein the method comprises determining the level of methylation of a first cell's DNA, and comparing the level to the DNA methylation level of a young cell, wherein when the level of DNA methylation of the first cell is less than the DNA methylation of the young cell, the first cell is identified as having an aged or old molecular status.

In certain embodiments, the DNA methylation level of the first cell is compared to DNA methylation reference standard, wherein the reference standard corresponds to a DNA methylation level of a young cell.

In certain embodiments, the methods for determining the molecular age of a cell comprise determining the ratio of expression levels of one or more Line1 (L1), LTR, and/or ERV repetitive elements to one or more ALU repetitive elements, wherein a ratio greater than 1 is indicative of the cell having an aged or old molecular status.

### 5.7 Methods for Reducing Aging

Within certain embodiments, the present disclosure provides methods for reducing aging and/or maturation of a cell, for example, an iPSC-derived cell, such as an iPSC-derived somatic cell (e.g., iPSC-derived fibroblasts and iPS cell-derived neurons), which methods include increasing the level of genomic nucleic acid methylation or other repressive marks, thereby reducing the expression level or presence in the cell of one or more chronological marker signatures and/or other age-related characteristics of an age-appropriate cell, such as a mature cell and/or an old cell, as described herein. Within some aspects of these embodiments, a marker signature and/or characteristic is associated with aging and/or one or more disease phenotype. In certain embodiments, the methods re-establish or increase genome-wide epigenetic silencing of gene expression, and a more youthful cellular state.

For example, cell type-specific chronological marker signatures can include, but are not limited to, a combination of one or more disease phenotype or chronological markers presented in Tables 1 and 2 and/or the absence of one or more of the chronological markers presented in Tables 1 and 2. Cell type-specific characteristics can include, but are not limited to, one or more phenotypes such as, for example, neuromelanin accumulation in aged iPSC-derived dopamine neurons. Disease phenotypes (related to Parkinson's disease) in neurons include, but are not limited to, pronounced dendrite degeneration, progressive loss of tyrosine-hydroxylase (TH) expression, and/or enlarged mitochondria or Lewy body-precursor inclusions. In certain embodiments, the present application provides for hypomethylation-induced aging of Parkinson's disease (PD)-iPSC-derived dopamine neurons to produce disease phenotypes that may be based upon genetic susceptibility.

The methods of the present invention can be applied to the production of young cells or youthful cells from somatic cells (whether iPSC-derived or primary cells) or from stem cells, or from fully differentiated or partially differentiated cells.

The present disclosure also provides: (1) methods for reducing maturation or aging in a cell and promoting youthfulness in the cell, including a somatic, a stem cell, and/or a stem cell-induced somatic cell displaying a marker signature of an “aged” or of a “mature” cell; (2) methods of therapeutic use of cells with reduced age prepared according to the methods described herein for treating a subject with, for example, a late-onset disease and/or disorder such as Parkinson's disease (PD); and to methods for using reduced aging in cell cultures (whether somatic or stem cell cultures, iPSC-derived or primary, or cells in the course of differentiation) to study chronological effects in late-onset diseases and/or disorders, such as Parkinson's disease (PD), in cultures of age-appropriate cells; and (3) iPSC-derived cells, including age-appropriate iPSC-derived cells, which produce one or more chronological markers or do not produce one or more chronological markers, the presence or absence of which chronological markers is characteristic of a chronological marker signature and/or a particular cellular phenotype (see, Tables 1-3).

In certain embodiments, the methods of the present application comprise contacting a cell with an agent that increases nucleic acid methylation in an amount and for a period of time sufficient to increase the level of nucleic acid methylation in the cell. In some embodiments, the cell can be a stem cell or a somatic cell. In a more particular embodiment, the cell can be an iPSC-derived cell. In a still more particular embodiment the iPSC-derived cell is a neuron. In certain embodiments, the iPSC-derived neuron is a midbrain dopamine neuron (mDA neuron). In certain embodiments, the iPSC-derived mDA neuron is derived from a subject with Parkinson's disease.

In certain embodiments, the cell is contacted with an agent that increases nucleic acid methylation in an amount and for a period of time sufficient to decrease expression of repetitive elements, for example, LINE1 and/or MIR elements.

In certain embodiments, the agent that increases nucleic acid methylation comprises a PIWI protein and/or a PIWI-interacting RNA (piRNA) and/or a somatic transposon protection factor APOBEC3B and/or a CRISPR nucleic acid.

In certain embodiments, the agent that increases nucleic acid methylation comprises a DNA methyltransferase (DNMT) and/or a histone methyltransferase (HMT) and/or a methyl-CpG-binding protein (MeCP2) and/or a PHD and RING finger domains 1 protein (UHRF1).

In certain embodiments, the increase in nucleic acid methylation achieved by the methods of the present application increases epigenetic silencing of DNA transcription, wherein such an increase of epigenetic silencing comprises an increase in the levels of repressive histone marks, for example, H3K9me3 and/or H3K27me3.

In certain embodiments, the increase in the level of nucleic acid methylation comprises an increase in the level of histone protein H1.

In certain embodiments, the increase in the level of nucleic acid methylation comprises an increase in the level of heterochromatin marker HP1α.

In certain embodiments, the increase in the level of nucleic acid methylation comprises an increase in the level of nuclear morphology marker LaminB1.

In certain embodiments, the increase in the level of nucleic acid methylation comprises a decrease in the level of a marker of DNA damage, for example, yH2Ax.

In certain embodiments, the agent that increases nucleic acid methylation comprises a sirtuin 1 (SIRT1) activator, for example, resveratrol. In certain embodiments, the agent is administered at a concentration of between about 1 and 50 μM, or any values in between, for example, between about 5 and 50 μM, or between about 10 and 50 μM, or between about 20 and 50 μM, or between about 30 and 50 μM, or between about 40 and 50 μM, or between about 1 and 40 μM, or between about 1 and 30 μM, or between about 1 and 20 μM, or between about 1 and 10 μM, or between about 1 and 5 μM.

In certain embodiments, the agent that increases nucleic acid methylation comprises an mTOR inhibitor, for example, rapamycin. In certain embodiments, the agent is administered at a concentration of between about 0.5 and 20 μM, or any values in between, for example, between about 1 and 20 μM, or between about 5 and 20 μM, or between about 10 and 20 μM, or between about 15 and 20 μM, or between about 0.5 and 15 μM, or between about 0.5 and 10 μM, or between about 0.5 and 5 μM, or between about 0.5 and 1 μM.

### 5.7.1 Reversing Age or Genetic Lesion in Age-Induced Pluripotent Stem Cell Derived Midbrain Dopamine Neurons

The present disclosure provides a pluripotent stem cell derived midbrain dopamine neuron cell in which aging has been induced. Disease phenotypes in matched iso-genic pairs of mutant and control lines can be employed to evaluate the effect of removing genetic susceptibility from cells.

A reversal of age phenotype can be monitored by: (i) decreased p-AKT activity, (ii) absence or reduction in dendrite degeneration compared to controls, or (iii) reduced rates of apoptosis compared to hypomethylated PD-iPSC derived DA neurons. Gene editing of the mutated gene resets age-related behavior.

### 5.8 Kits

The presently disclosed subject matter provides for kits for inducing aging and/or maturation of a cell, for example, an iPSC-derived cell, such as an iPSC-derived somatic cell (e.g., iPSC-derived fibroblasts and iPSC-derived neurons), wherein the aged cell expresses one or more chronological markers of an aged cell. In certain embodiments, the kit comprises one or more inhibitors of nucleic acid methylation, and instructions for inducing age in the cell, such that the cell expresses one or more chronological markers of an aged cell.

In certain embodiments, the instructions comprise contacting the cell with the inhibitor(s) in an amount effective to decrease the level of DNA methylation in the cell.

The presently disclosed subject matter provides for kits for reducing aging and/or maturation of a cell, for example, an iPSC-derived cell, such as an iPSC-derived somatic cell (e.g., iPSC-derived fibroblasts and iPSC-derived neurons), wherein the expression of one or more chronological markers of age in the cell is decreased following a reduction in the cell's age. In certain embodiments, the kit comprises one or more agents that induces or increases nucleic acid methylation, and instructions for reducing age in the cell, such that the expression of one or more chronological markers of an aged cell are decreased in the cell following treatment of the cell according to the instructions.

In certain embodiments, the instructions comprise contacting the cell with the agent (s) in an amount effective to increase the level of DNA methylation in the cell.

In certain embodiments, the kit comprises instructions for administering a population of the presently disclosed cells, for example, stem-cell-derived neurons, such as midbrain dopamine neurons, or precursors thereof, or a composition comprising said cells, to a subject suffering from a disorder, such as a neurological disorder, for example, Parkinson's disease or Alzheimer's disease. The instructions can comprise information about the use of the cells or composition for treating or preventing the disorder. In certain embodiments, the instructions comprise at least one of the following: description of the therapeutic agent; dosage schedule and administration for treating or preventing the disorder, or symptoms thereof; precautions; warnings; indications; counter-indications; over dosage information; adverse reactions; animal pharmacology; clinical studies; and/or references. The instructions can be printed directly on a container (when present) comprising the cells, or as a label applied to the container, or as a separate sheet, pamphlet, card, or folder supplied in or with the container.

### 6. EXAMPLES

The presently disclosed subject matter will be better understood by reference to the following Example, which is provided as exemplary of the presently disclosed subject matter, and not by way of limitation.

### Example 1

### Directed Differentiation of Neuronal Cell Types

This example describes one method of directed differentiation techniques to generate specific neural cell types. Nearly pure populations of CNS lineages, such as midbrain dopamine (mDA) neurons, are used in the methods described herein. The protocol of Kriks et al, Nature 2011, infra, can be used (among other methods).

Briefly, a modified version of the dual-SMAD inhibition protocol can be used to direct cells towards floor plate-based mDA neurons as described previously (Kriks et al., Nature 480:547-551 (2011). iPSC-derived mDA neurons can be replated on day 30 of differentiation at 260,000 cells per cm² on dishes pre-coated with polyornithine (PO; 15 μg/ml)/Laminin (1 μg/ml)/Fibronectin (2 μg/ml) in Neurobasal/B27/L-glutamine-containing medium (NB/B27; Life Technologies) supplemented with 10 μM Y-27632 (until day 32) and with BDNF (brain-derived neurotrophic factor, 20 ng/ml; R&D), ascorbic acid (AA; 0.2 mM, Sigma), GDNF (glial cell line derived neurotrophic factor, 20 ng/ml; R&D), TGFβ3 (transforming growth factor type (33, 1 ng/ml; R&D), dibutyryl cAMP (0.5 mM; Sigma), and DAPT (10 nM; Tocris,). One to two days after plating, cells can be treated with 1 μg/ml mitomycin C (Tocris) for 1 hour to kill any remaining proliferating contaminants. iPSC-derived mDA neurons can be fed every 2 to 3 days and maintained without passaging until the desired timepoint for a given experiment. PO, laminin and fibronectin can be added to the medium every 7-10 days to prevent neurons from lifting off.

### Example 2

### Profiling of mRNA, 5hMC and DNA Methylation

This example describes one technology to profile mRNA, 5hMC and DNA methylation in the presently described age paradigm. These methods provide data regarding the molecular control of age-related factors.

5-mC Detection

An enhanced reduced-representation bisulfite sequencing (ERRBS) method may be used. In this protocol, genomic DNA is digested by Msp1 restriction enzyme and fragments are size selected to obtain fragments enriched for CpG sites. These fragments undergo bisulfite conversion, sequenced on Illumina HiSeq200035 and the sequencing data are analyzed by custom software that maps bisulfite-treated sequencing reads and outputs to the methylation status of identified CpG sites.

5-hmC Detection

A Hydroxymethyl Collector™ kit from Active Motif may be used. This protocol is based on the selective addition of a biotin moiety to 5-hmC positions followed by an immunoprecipitation (IP) step. Similar to ChIP-seq experiments, both the total cellular input and IP fragments are sequenced. 5-hmC modifications are identified as regions of high coverage over background levels.

Gene Expression Detection

The RNA-seq protocol may be used. This protocol is well known in the art and is routinely performed at the WCMC epigenomics core. Sequencing experiments will be multiplexed to reduce sequencing cost and to prevent batch effects.

### Example 3

### Gene Corrected PD-iPSC Lines

This example describes the use of a gene corrected PD-iPSC line (e.g., TALEN-based gene targeting). Although it is not necessary to understand the mechanism of a disclosure, it is believed that these cell lines provide access to iso-genic pairs of PD-iPSC and control iPSC to more precisely distinguish between disease factors related to age and factors related to genetic susceptibility to PD.

### Example 4

### Alternative Differentiation of Induced Pluripotent Stem Cells into Midbrain Dopamine Cells

Alternatively, neural differentiation of iPSC can be initiated using a modified version of the dual-SMAD inhibition (Chambers et al., Nat. Biotechnol. 27:275-280 (2009), herein incorporated by reference). Floor plate induction (Fasano et al., Cell Stem Cell 6:336-347 (2010), herein incorporated by reference) protocol can be used based on timed exposure to LDN-193189 (100 nM (ranging in concentration from 0.5-50 μM, Stemgent, Cambridge, Mass.), SB431542 (10 μM (ranging in concentration from 0.5-50 μM, Tocris, Ellisville, Mich.), SHH C25II (100 ng/ml (ranging in concentration from 10-2000 ng/ml, R&D, Minneapolis, Minn.), Purmorphamine (2 μM (ranging in concentration from 10-500 ng/ml, Stemgent), FGF8 (100 ng/ml (ranging in concentration from 10-500 ng/ml, R&D) and CHIR99021 (CHIR; 3 μM (ranging in concentration from 0.1-10 μM, Stemgent). “SHH” treatment refers to exposure, i.e. contact, of cells to a combination of SHH C25II 100 ng/ml+Purmorphamine (2 μM).

Cells can be plated (35-40×10³ cells/cm²) and cultured on matrigel or geltrex (used as purchased) (BD, Franklin Lakes, N.J.) in Knockout serum replacement medium (KSR) containing DMEM, 15% knockout serum replacement, 2 mM L-glutamine and 10-μM (ranging in concentration from 1-25 μM β-mercaptoethanol. KSR medium gradually shifted to N2 medium starting on day 5 of differentiation, by mixing in ratios of 75% (KSR):25% (N2) on day 5-6, 50% (KSR):50% (N2) day 7-8 and 25% (KSR):75% (N2) on day 9-10, as described previously (Chambers et al., Nat. Biotechnol. 27:275-280 (2009), herein incorporated by reference).

On differentiation day 11, media can be changed to Neurobasal medium/B27medium (1:50 dilution)/L-Glut (effective ranges 0.2-2 mM)) containing medium (NB/B27; Invitrogen) supplemented with CHIR (until day 13) and with BDNF (brain-derived neurotrophic factor, 20 ng/ml ranging from 5 to 100; R&D), ascorbic acid (AA; 0.2 mM (ranging in concentration from 0.01-1 mM), Sigma, St Louis, Mo.), GDNF (glial cell line-derived neurotrophic factor, 20 ng/ml (ranging in concentration from 1-200 ng/ml); R&D), TGFβ3 (transforming growth factor type (33, 1 ng/ml (ranging in concentration from 0.1-25 ng/ml); R&D), dibutyryl cAMP (0.5 mM (ranging in concentration from 0.05-2 mM); Sigma), and DAPT (10 nM (ranging in concentration from 0.5-50 nM); Tocris,) for 9 days.

On day 20, cells can be dissociated using Accutase® (Innovative Cell Technology, San Diego, Calif.) and replated under high cell density conditions (for example from 300-400 k cells/cm²) on dishes pre-coated with polyornithine (PO); 15 μg/ml (ranging in concentration from 1-50 μg/ml)/Laminin (1 μg/ml) (ranging in concentration from 0.1-10 μg/ml)/Fibronectin (2 μg/ml (ranging in concentration from 0.1-20 μg/ml) in differentiation medium (NB/B27+BDNF, AA, GDNF, dbcAMP (ranging in concentration as described herein), TGFβ3 and DAPT (ranging in concentration as described herein) until the desired maturation stage for a given experiment.

### Example 5

### Immunocytochemical Analyses

A list of antibodies and concentrations is provided in Table 5 below that can be used for detecting chronological markers. These antibodies can be used for detecting chronological markers by techniques including electronic microscopy (EM); flow cytometry (FC); immunocytochemistry (ICC); IHC, immunohistochemistry (IHC); western blot (WB), among others.

TABLE 5
Chronological Marker-specific Antibodies

| Antigen | Company | Host | Concentration |
|---|---|---|---|
| p-4EBP1 | Cell Signaling | Rabbit | 1:1000 (WB) |
| 4EBP1 (total) | Cell Signaling | Rabbit | 1:1000 (WB) |
| p-AKT | Cell Signaling | Rabbit | 1:250 (WB) |
| AKT (total) | Cell Signaling | Rabbit | 1:1000 (WB) |
| CD13-PE | BD | | 20 μl per |
| | | | 1M cells (FC) |
| Cleaved caspase-3 | Cell Signaling | Rabbit | 1:100 (ICC) |
| FOXA2 | Santa Cruz | Goat | 1:200 (ICC) |
| GFP | Abcam | Chick | 1:2000 (WB, IHC) |
| GFP | Aves | Chick | 1:3000 (EM) |
| γH2AX | Millipore | Mouse | 1:250 (ICC) |
| H3K9me3 | Abcam | Rabbit | 1:4000 (ICC) |
| HLA-ABC-APC | BD | | 20 μl per |
| | | | 1M cells (FC) |
| HP1γ | Millipore | Mouse | 1:200 (ICC) |
| Ki67 | Dako | Mouse | 1:100 (ICC) |
| Lamin A | Abcam | Rabbit | 1:100 (ICC) |
| Lamin A/C | Abcam | Mouse | 1:200 (ICC) |
| (clone JOL2) | | | |
| Lamin A/C | Santa Cruz | Goat | 1:100 (WB) |
| (clone N-18) | | | |
| Lamin B2 | Abcam | Mouse | 1:500 (ICC) |
| Lamin C | Abcam | Rabbit | 1:100 (ICC) |
| LAP2α | Abcam | Rabbit | 1:500 (ICC) |
| LMX1A | Millipore | Rabbit | 1:2000 (ICC) |
| MAP2 | Sigma | Mouse | 1:200 (ICC) |
| NANOG | R&D | Goat | 1:50 (ICC) |
| Nestin | R&D | Mouse | 1:300 (ICC) |
| NURR1 | R&D | Mouse | 1:1000 (ICC) |
| OCT4 | Santa Cruz | Mouse | 1:200 (ICC) |
| Sendai | MBL Int. | Rabbit | 1:500 (ICC) |
| SSEA3-FITC | BD | | 20 ul per |
| | | | 1M cells (FC) |
| SSEA4-PE | BD | | 20 ul per |
| | | | 1M cells (FC) |
| Total AKT | Cell Signaling | Rabbit | 1:500 (WB) |
| TUJ1 | Covance | Mouse/ | 1:500 (ICC) |
| | | Rabbit | |
| Tyrosine hydroxylase | Pel-Freez | Rabbit | 1:500 (ICC, |
| (TH) | | | IHC, WB) |

### Example 6

### Fibroblast Differentiation

Differentiation of iPSCs to fibroblast-like cells can be based on a protocol from Park et al., Nature 141-146 (2008)). Briefly, iPSC clones can be enzymatically passaged using dispase and plated as multicell clumps onto gelatin in iPSC maintenance medium that had been conditioned on MEFs for 24 hours and then supplemented with 10 ng/ml FGF₂ and 10 μM Y-27632. The next day the medium can be replaced with Minimal Essential Medium Alpha (Life Technologies) supplemented with 15% fetal bovine serum (Life Technologies) and continually changed every other day thereafter. The differentiating cells can be carefully passaged every 5-6 days using Accutase (Innovative Cell Technology, San Diego, Calif.) for the first two weeks and then trypsinized subsequently. Y-27632 can be added to the medium on the day of passaging to help support attachment. After four weeks fibroblast-like cells can be sorted based on high expression levels of CD-13 and HLA-ABC prior to phenotype assessment and overexpression studies. Sorted cells can be expanded in Minimal Essential Medium Alpha with 15% fetal bovine serum (no Y-27632) thereafter.

### Example 7

### mDA Neuron Differentiation

This example contains a longer version of the protocol of Example 1. A modified version of the dual-SMAD inhibition protocol can be used to direct cells towards floor plate-based mDA neurons as described previously (Kriks et al., Nature 480:547-551 (2011)).

iPSC-derived mDA neurons can be replated on day 30 of differentiation at 260,000 cells per cm2 on dishes pre-coated with polyornithine (PO; 15 μg/ml)/Laminin (1 μg/ml)/Fibronectin (2 μg/ml) in Neurobasal/B27/L-glutamine-containing medium (NB/B27; Life Technologies) supplemented with 10 μM Y-27632 (until day 32) and with BDNF (brain-derived neurotrophic factor, 20 ng/ml; R&D), ascorbic acid (AA; 0.2 mM, Sigma), GDNF (glial cell line-derived neurotrophic factor, 20 ng/ml; R&D), TGFβ3 (transforming growth factor type β3, 1 ng/ml; R&D), dibutyryl cAMP (0.5 mM; Sigma), and DAPT (10 nM; Tocris,).

One to two days after plating, cells can be treated with 1 μg/ml mitomycin C (Tocris) for 1 hour to kill any remaining proliferating contaminants. iPSC-derived mDA neurons can be fed every 2 to 3 days and maintained without passaging until the desired timepoint for a given experiment. PO, laminin and fibronectin can be added to the medium every 7-10 days to prevent neurons from lifting off.

### Example 8

### Assessment of Senescence

Senescence-activated beta-galactosidase can be assessed using the staining kit from Cell Signaling according to the manufacturer's instructions. Positive cell staining was manually assessed (2 replicates, 50 cells each).

Telomere Length Measurements by HT-QFISH

Cells were plated on a clear-bottom, black-walled, 96-well plate, including 4 well replicates per sample, and high throughput quantitative fluorescence in situ hydridization (HT-QFISH) was performed as previously described (Canela et al., Proc Natl Acad Sci USA 104:5300-5305 (2007)). Images were captured with the Operetta using a 20× objective. Image processing was performed using Harmony high content analysis software. Telomere length values were measured using individual telomere spots corresponding to the specific binding of a Cy3-labeled telomeric probe (>600 spots per sample) in quadruplicate samples, fluorescence intensities were converted into kilobases using control cell lines of known telomere length as described previously (Canela et al., Proc Natl Acad Sci USA 104:5300-5305 (2007) and McIlrath et al., Cancer research 61: 912-915 (2001)).

### Example 9

### Method for Screening Drugs Using Age-Modified Cells

iPSC can be obtained, for example, from human fibroblasts by methodology that is disclosed herein and as otherwise known in the art. Age-modified somatic cells can be obtained from iPSC by differentiation and reduction of genomic nucleic acid methylation. Specialized age-modified somatic cells can thus be obtained having the characteristics of somatic cells isolated from brain, heart, liver, kidney, spleen, muscle, skin, lung, blood, artery, eye, bone marrow, and the lymphatic system. Differentiation protocols yielding such somatic cells are known, including cardiomyocytes (See, e.g., Van Oorschot A A et al., Panminerva Med. 2010 June; 52(2):97-110), hepatocytes (See, e.g., Alaimo G. et al., J Cell Physiol. 2013 June; 228(6): 1249-54), kidney cells (See, e.g., De Chiara L. et al., J Am Soc Nephrol. 2014 February; 25(2):316-28), pancreatic beta cells (See, e.g., Roche E. et al., J Stem Cells. 2012; 7(4):211-28), white blood cells (See, e.g., de Pooter R F et al., Methods Mol Biol. 2007; 380:73-81).

Once cells are ready for screening, they can be plated to test various plating densities and cell culture vessels. For example, these cells can be plated on 6-well, 24-well, 96-well, 384-well plates or any other platforms that facilitate drug screening. Times for initiation and duration of trophic factor withdrawal will also be optimized once a suitable HTS format is selected.

Drug screens based on stem-cell derived somatic cells have been described. See, e.g., Yang et al., Cell Stem Cell 12:713-726 (2013). Briefly, a small molecule survival screen was carried out using iPSC-derived motor neurons (MNs) from both wild-type and mutant SOD1 mouse embryonic stem cells to search for drugs to counteract MN death in amyotrophic lateral sclerosis (ALS). Mouse ESCs were differentiated into MNs and plated in 96-well or 384-well plates. Additionally, human MNs derived from human ESCs and iPSCs after 30 days of differentiation, were also used. For the small molecule screen, freshly dissociated cells were plated at a density of 8,000 GFP+ cells (384-well plate) or 30,000 GFP+ cells (96-well plate) per well. Four days later, trophic factors were removed, and individual compounds were added to the wells. For the primary screen each compound was tested at three concentrations (0.1 mM, 1 mM, and 10 mM) in duplicate. After an additional 72 hr (day 7), cells were fixed and stained, and the number of MNs surviving was analyzed by counting the remaining GFP+ cells in the whole well. Survival is measured as fold increase compared to cultures maintained without trophic factors. Using this method, Yang and colleagues discovered that the compound kenpaullone had an impressive ability to prolong the healthy survival of MNs.

By combining age-modification methods described in the present disclosure and an HTS platform, drug screening can be performed on cells that represent late-onset human diseases. According to methods of the present disclosure, age-modified cells with appropriate age and/or maturation markers can be generated from a somatic cell or from a stem cell. For example, an age-appropriate iPSC-derived mDA neuron can be generated by reducing the level of genomic nucleic acid methylation in an iPSC derived neuron. Cells to be tested in a drug screen can be plated to test various plating densities and cell culture vessels. For example, cells can be plated on 6-well, 24-well, 96-well, 384-well plates or any other platforms that facilitate the drug screening. Times for initiation and duration of trophic factor withdrawal will also be optimized once a suitable HTS format is selected.

Molecules for use in a drug screen can come from a variety of sources, including small molecule compound libraries that can be designed in-house or obtained commercially. In the case of age-appropriate iPSC-derived mDA neurons, known drug molecules for neurodegenerative diseases, such as Parkinson's disease, which include biological and small molecules, can be tested. Such molecules can be screened at different concentrations, in combination with different cell densities, to optimize drug screen efficacy. For example, Yang et al. screened a collected of approximately 5000 small molecules to search for an ALS drug. For the primary screen, each compound was tested at three concentrations (0.1 mM, 1 mM, and 10 mM) in duplicate. After an additional 72 hr (day 7), MN cells were fixed, stained and accessed for survival. Yang et al., Id.

The phenotypic changes of the age-modified cells after exposure to candidate compounds (whether small molecules or biologics) can be selected according to the disease intended to be treated as well as according to the intended effects of these compounds/molecules on these cells. These phenotypic changes include, but not limited to, cell survival, morphological changes of the cells, secretion of certain factors by the cells, expression of certain cell surface molecules, interaction of cells with other cells and/or with a solid support, changes in optical, electrical, and chemical properties of the cell, fluorescence signals of the cell (e.g., when the cells are transfected with a fluorescent protein) and attenuation or elimination of disease markers, among others. One application of the methods described by the present disclosure is to screen for drugs that can prolong the healthy survival of neuronal cells that are key to neurodegenerative diseases such as Parkinson's and Alzheimer's diseases. Thus, a drug screen can be designed to select compounds that will promote survival of neurons. In the case of PD, age-modified mDA neurons derived from iPSC can be cultured, plated and exposed to compounds and their survival rate accessed. Furthermore, additional markers can be utilized as a basis for the drug screen in addition to cell survival. For example, aging/maturation-related markers, such as those listed in Table 2 or Table 3, can be used as criteria for drug screens. Compounds that can slow, halt or reverse the expression of one or more aging or disease markers could be candidates for drugs that may help treat these neurodegenerative diseases.

Hits can be defined as compounds/molecules that will effectively reverse one or more age-related or disease-related marker signatures described above. For example, if cell survival is used and an endpoint, molecules can be selected that substantially increase the number of surviving cells (e.g., age-appropriate iPSC-derived mDA neurons) while preserving cell-appropriate morphological characteristics.

Candidate compounds that are selected from a primary screen can, optionally, be retested and subjected to additional testing including, but not limited to, dose-response and toxicity assays. Lead compounds can be selected and can be structurally modified to improve desired characteristics and/or to reduce side effects. Other improvements to the lead compounds can include increased absorption, longer half-life, higher affinity to cells, and enhancement of local and/or systemic delivery. Lead compounds and modified variants thereof can be further studied in preclinical studies including in suitable cell culture and animal model systems and, those exhibiting favorable therapeutic and toxicity profiles can be subjected to further in vivo testing in human clinical trials.

### REFERENCES

Akalin et al., Genome Biol 13:R87 (2012)
Akalin et al., PLoS Genet 8:e1002781 (2012)
Anders & Huber, Genome Biol 11:R106 (2010)
Cerami et al., PLoS One 5:e8918 (2010)
Hetman & Pietrzak, Trends Neurosci 35:305-314 (2012)
Johnson et al., Curr Opin Cell Biol 10:332-338 (1998)
McCord et al., Genome Res 23:260-269 (2013)
Nuytemans et al., Hum Mutat 31:763-780 (2010)
Rieker et al., J Neurosci 31:453-460 (2011)
Ross-Innes et al., Nature 481:389-393 (2012)
Sinclair et al., Science 277:1313-1316 (1997)
Stroud et al., Genome Biol 12:R54 (2011)
Studer, et al., Proc Natl Acad Sci 106:12759-12764 (2009)
Tarca et al., Bioinformatics 25:75-82 (2009)
Vera et al., Cell Rep 2:732-737 (2012)

### Example 10

### Inducing Age in Cells by Hypomethylation

### Introduction

A fundamental step towards developing new therapies for incurable conditions is the study of disease-affected tissues. Until recently, this was hampered by the scarce availability of biopsies, in particular for brain disorders, such as Parkinson's (PD) or Alzheimer's disease (AD). Thanks to the revolutionary technology of induced pluripotent stem cells (iPSC), it is now possible to produce any cell type of the body, as an inexhaustible source of cells to study disease in the laboratory. This technique, termed “disease-modeling”, is used to analyze disease mechanisms in a dish and for screening new drugs on the “in-vitro version” of a patient, helping to predict efficacy or side effects.

Numerous studies have shown that iPSC are a powerful tool for this purpose, however, for now, only conditions of early childhood can be faithfully be recreated, whereas modeling disorders of old age, does not recapitulate the crucial symptoms occurring in patients. The source of this problem is intrinsic to the iPSC-method, which consists of reprogramming adult cells back to an embryonic state. This renders cells capable of producing any tissue of the body but simultaneously, it reverses their biological clock to a very young stage, equivalent to a newborn. It has recently been shown that cells derived from iPSC are rejuvenated compared to the original cells from old donors. While this phenomenon opens the compelling possibility of restoring cellular youth in old cells, it precludes the use of iPSC-technology for age-dependent diseases, as cells created from iPSC are too young to manifest age signs.

In light of these facts, it is proposed herein to elucidate for the first time how rejuvenation following reprogramming is encoded in the DNA, and in parallel, to develop a method for the acceleration of natural aging in a dish, in order to advance signs of age and disease symptoms in cells derived from iPSC.

There has been recent success in fast-forwarding cellular age through a disease-factor responsible for premature aging (progeria). This allowed for the first time to recreate in a dish those cellular anomalies seen in brains of PD patients. However, new data suggest that there may be a molecular discrepancy between premature and real aging, indicating that using a disease-factor might compromise the interpretation of experimental results.

Improvements in the methodology by mimicking a natural aging process observed in normal cells of most tissues is described herein. This mechanism is the loss of DNA methylation, a factor responsible for the correct functioning of the nuclear machinery and thus all cellular processes. The proven relevance of this aspect for age in different organs should render this technique applicable to a variety of cell types and diseases.

### SUMMARY

The advent of induced pluripotent stem cells (iPSC) has revolutionized the study of disease, allowing for the in-vitro generation of patient-specific cells to study disease mechanisms and for drug screening. At the same time, it provides a powerful biological paradigm to investigate the definition and malleability of cell identity. Differentiation of iPSC into various lineages creates cells of fetal-like nature that have re-attained a youthful state. Understanding this process might reveal the molecular mechanisms that control and possibly reverse cellular age. Conversely, this phenomenon represents a barrier for mimicking aspects of old age in iPSC-derived cells and thus for modeling late-onset diseases.

One embodiment of the present example is to elucidate the genomic processes that dictate cellular rejuvenation through reprogramming by transcriptomic and epigenetic profiling of primary cells of different donor ages and their fate-matched iPSC-derived progeny. This should both yield a comprehensive set of new molecular markers for the measurement of biological aging, as well as potentially indicate which factors could be manipulated to reverse cellular age.

Second, a novel strategy is proposed to accelerate age in-vitro, aimed at facilitating the modeling of age-dependent disorders with iPSC-technology. It has been previously demonstrated that expression of progerin was able to induce age-related phenotypes from iPSC-derived dopamine neurons. (See International Publication No. WO/2014/172507, published Oct. 23, 2014, which is incorporated by reference in its entirety for all purposes). The approach described by the present example aims to uncouple the disease-related component brought by the expression of progerin from the aging component. To improve the methodology for aging cells, the present example recapitulates a naturally occurring, age-dependent event, which is gradual loss of DNA methylation. Given the conserved role of DNA hypomethylation for aging in different tissues and cell types, such efforts may provide a simple tool to fast-track biological aging in multiple lineages.

The rejuvenating effects that accompany cellular reprogramming represent a fascinating but marginally explored territory of stem cell biology. At the same time, this phenomenon creates an obstacle to the use of iPSC for the study of late-onset diseases. Several groups have tried to bypass this limitation by challenging cells with damaging agents or environmental stressor in order to trigger pathological cell behaviors^{21,22}. While for many diseases, e.g. PD, the exposure to environmental toxins is a major risk factor, acute treatment of cells does not recapitulate real disease ontogeny and might therefore not yield truly significant results.

It has been shown that acceleration of cellular age is feasible through expression of progerin and can elicit previously unidentified degenerative phenotypes in iPSC-based models of PD, proving the concept that inducing age is necessary to promote age-dependent phenotypes in iPSC-derived cells. Preliminary data however reveals that progeria does not share transcriptional and epigenetic features of normal age, indicating that expression of progerin might not faithfully reflect physiological aging. Such a technical bias could potentially lead to artifactual results and misinterpretations.

The present example characterizes the molecular dynamics of aging and cellular rejuvenation in detail, using cutting edge technology and a unique set of primary and iPSC-derived isogenic cell lines from different donor-ages. This has the potential to identify novel molecular markers of physiological aging and to elucidate the molecular signature that governs the rejuvenated state of stem cells, a fundamental, unanswered question in the stem cell field. The experiments seek a causative link between global hypomethylation and aging which would have profound effects on the aging field, demonstrating active involvement of DNA methylation in the aging processes. The methods described herein may therefore provide a simple tool for the manipulation of cellular age in vitro, to allow for more accurate in vitro models of age-dependent diseases via iPSC-technology.

### 1. Genomic Profiling of Cellular Rejuvenation Through Reprogramming

Understanding and reversing the inexorable process of aging is an ancient dream of mankind. Evidence shows that the pace of aging can be manipulated by different interventions^{2, 3}. Yet, only few processes are able to reverse an aged state back to a more youthful state, among these is reprogramming cells to pluripotency³. In fact, the generation of induced pluripotent stem cells (iPSC) not only rewinds the biological clock from a developmental perspective, but also erases features of cellular age³⁻⁶. Compelling evidence has recently been provided showing that re-differentiation of iPSCs into various lineages leaves cells “rejuvenated” by resetting numerous hallmarks of biological aging′. A number of studies has described different aspects of this phenomenon, however these mostly focus on phenotypic comparisons of cellular features before and after reprogramming⁷⁻⁹. While it has been suggested that pluripotency might restore cellular youth through epigenetic mechanisms^{4,10}, an in-depth genomic analysis of this process has not been reported. Understanding how rejuvenation is encoded in the genome could provide invaluable knowledge on the molecular determinants of age and open the possibility of devising methods for reprogramming cellular age independently of cellular fate.

In this light, this working example generates a comprehensive representation of how transcriptional and epigenetic features that define cellular age are remodeled after reprogramming and re-differentiation into the same cell type to restore a youthful identity. This is achieved by genomic profiling of primary fibroblasts from donors of different ages and their iPSC-derived fibroblast progeny. The strength of this approach lies in the unique advantage of iPSC-technology to reverse cellular age, while restoring cellular fate, thereby comparing isogenic cells prior and after reprogramming and eliminating the effect of genetic variability.

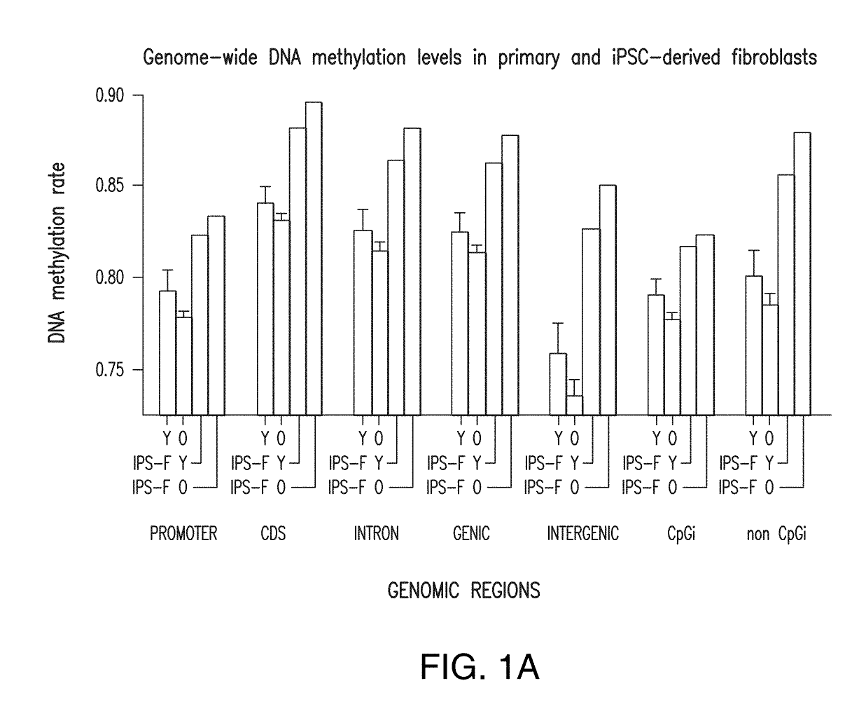

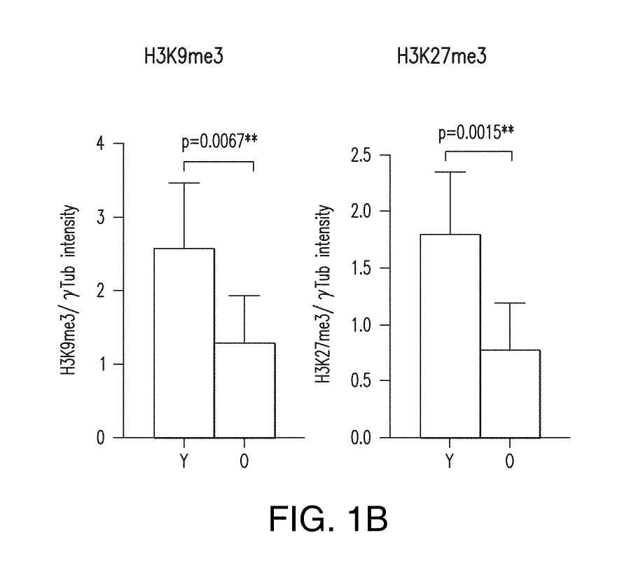

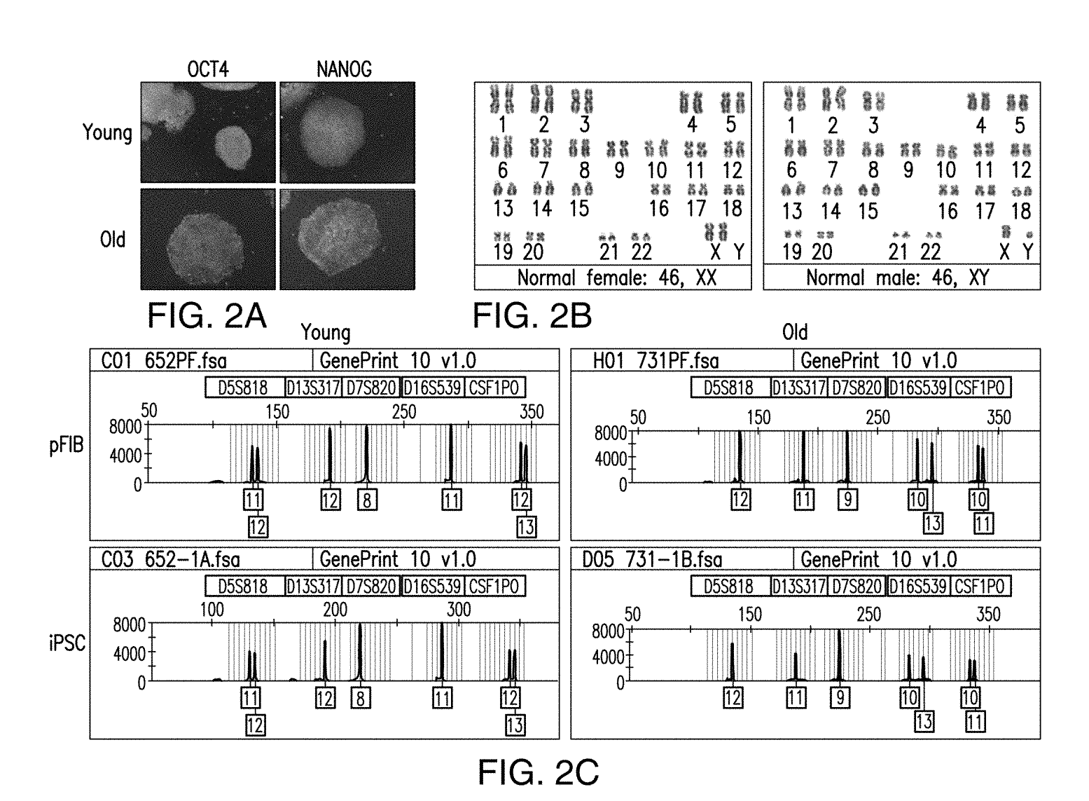

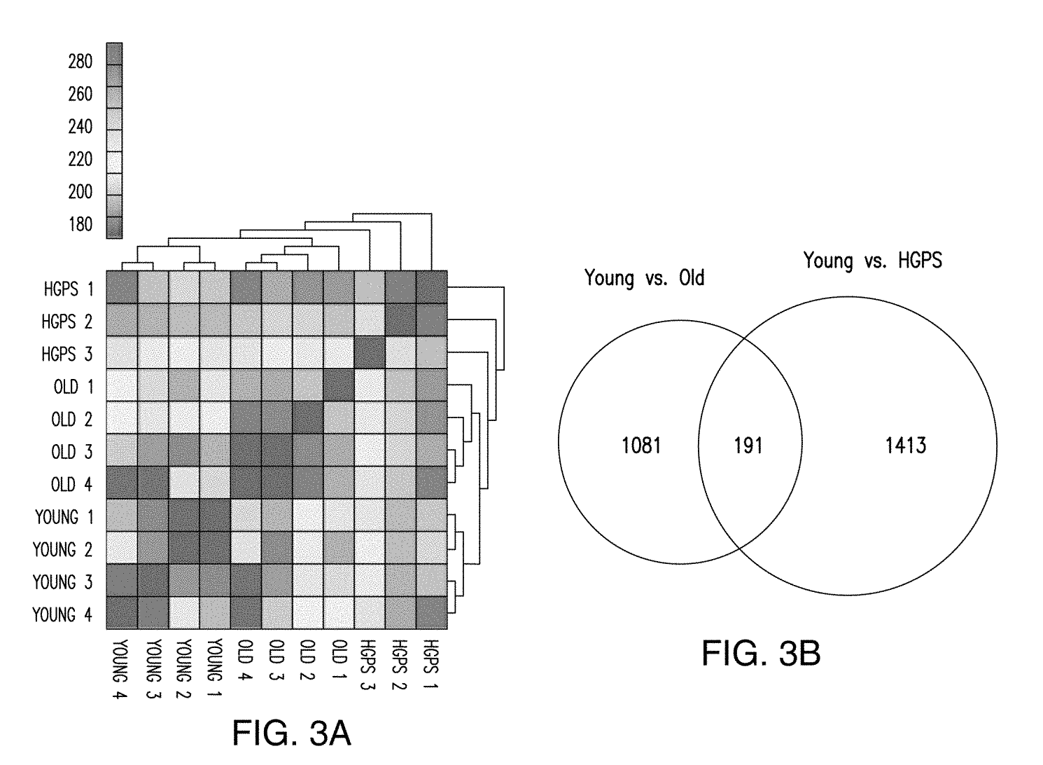

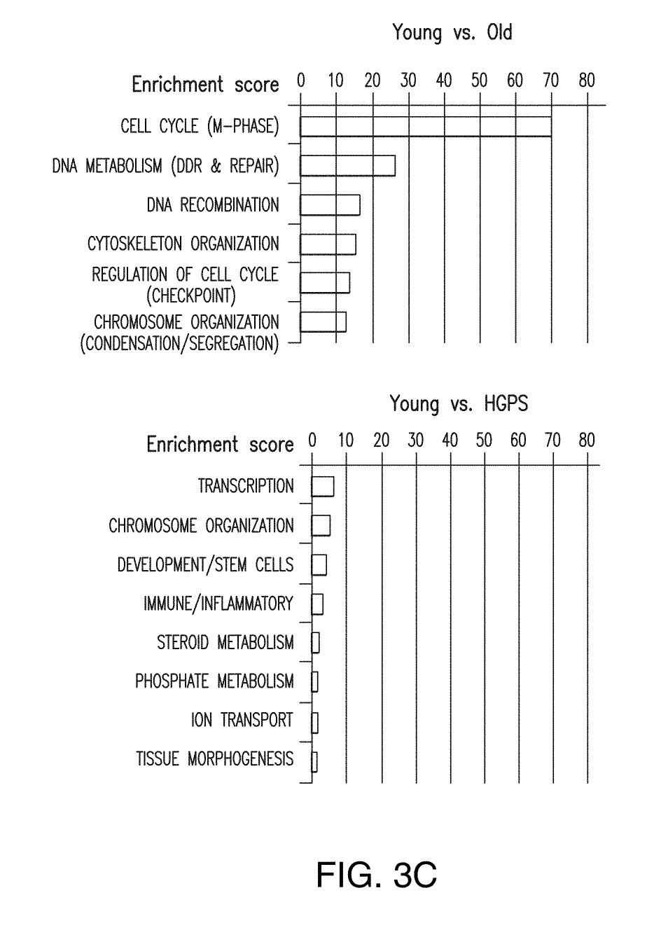

A cohort of primary fibroblast lines was obtained from three age groups: young, middle-aged and old. iPSCs were generated and validated (FIG. 2) for 3 young, 2 middle aged, 4 old lines, and further iPSC derivation is underway. Differentiation of iPSCs back into fibroblasts follows an established protocol and the attainment of bone fide fibroblasts was validated by cell surface marker expression⁷. Transcriptomic and DNA methylation profiles were generated for all primary fibroblasts (RNA-Seq and ERRBS, respectively). ChIP-seq of major histone modifications with a reported role in aging¹¹ (H3K9me3, H3K27me3, H3K4me3, H3K36me3) is being optimized. Initial transcriptomic data of old and young primary cells shows a clear age-related segregation, indicating differential regulation of specific pathways between young and old cells (FIG. 3a). Aging has been associated with defined changes in the epigenetic landscape, in particular DNA hypomethylation and loss of repressive histone modifications^{11,12}. In agreement with this, the present data confirms decreased genome-wide levels of DNA methylation and the repressive marks H3K9me3 and H3K27me3 in aged cells (FIG. 1). Interestingly, preliminary results demonstrate that methylation levels are restored in iPSC derived fibroblasts of both young and old donors (FIG. 1A). This exciting observation provides a first molecular hint to how cellular age might be reversed upon reprogramming and high levels of methylation in iPSC-derived cells could suggest their fetal nature. Given the robustness of aging related changes in DNA methylation, independent groups have proposed the use of specific CpG sites as “epigenetic age predictors”¹³⁻¹⁶. In particular, a recent study reveals the existence of 353 CpG sites, whose methylation state strictly correlates with chronological age across all tested human tissues and cell types¹⁴. To incorporate this “molecular age marker” into the present example, a customized methylation platform was developed and is currently being tested.

5-hydroxy-methyl-cytosine (5hmC) is a recently discovered DNA modification with a reported role in pluripotency and aging in the brain¹⁶. 5hmC profiles from primary young and old cells are currently being generated, which may provide insight into a possible age-dependent role of this novel epigenetic mark in a somatic, non-neuronal cell type.

The findings of the present example may identify global molecular features defining the aged state in primary cells and elucidate how this signature is lost, entirely or partially, upon reprogramming and re-differentiation into the same cell type. This paradigm bears the unprecedented benefit of directly comparing isogenic lines of the same identity before and after reprogramming, excluding the effect of genetic diversity. Such a method may uncover mechanisms of rejuvenation that could be applied to attempts at uncoupling a reversal of cellular age from cellular fate. Finally, a set of molecular age markers identified by the methods described herein may serve as a tool to measure biological age in the context of efforts to induce aging in-vitro.

Additionally, the present example compares transcriptomes of iPSC-derived fibroblasts and primary fetal fibroblasts. Such a comparison may compensate for any imperfect restoration of fibroblast identity in iPSC-derived fibroblasts that may interfere with the comparative analysis aimed at identifying genomic changes signifying age.

2. Improved In-Vitro Aging Strategy to Induce Physiological Age in iPSC-Derived Lineages

In the last years, iPSC-technology has proven a revolutionary tool for the study of human disease and the discovery of novel therapeutic targets. In fact, a series of successful studies supports the potential of iPSC-based disease modeling for a variety of disorders. However, until recently only developmental or juvenile pathologies could be faithfully recreated in vitro using patient-specific iPSC¹⁷⁻²⁰. In fact, modeling of age-dependent conditions, such as neurodegenerative disorders do not reproduce the characteristic degenerative phenotypes, though several reports suggest the presence of early stage molecular and biochemical disease indicators²¹⁻²³. Lack of degenerative features may be due to the immature or youthful nature iPSC-derived cells and hence that modeling of late-onset diseases may require the implementation of “age” to deliver truly relevant results. This is supported by recent work, which for the first time succeeded in eliciting degenerative features in iPSC-derived midbrain dopamine neurons (mDA) from Parkinson's disease (PD) patients, by accelerating cellular age through expression of progerin⁷. Progerin is a mutant protein responsible for a severe form of premature aging, known as Hutchinson Gilford Progeria Syndrome (HGPS)²⁴ and cells derived from HGPS patients display many phenotypic age markers seen in cells from old donors. However, in spite of the striking resemblance of HGPS children to old individuals, it remains uncertain whether progeria is equivalent to “true” aging from a molecular perspective²⁵. The risk of employing progerin in iPSC models of disease is a potential bias deriving from the use of a disease-factor. To investigate whether shared molecular features determine the phenotypic similarity between progeria and old cells, gene expression and DNA methylation profiles of primary young, old and progeria fibroblasts were compared. In contrast to the numerous phenotypic marks, that are widely shared between aged and progeria cells⁷, transcriptomic (FIG. 3) and epigenetic profiles (data not shown) of progeria cells were significantly different from both young and old healthy cells. This new data argues that at the global genomic level, progeria cells do not align closely to old cells. This discrepancy is further reflected in the limited overlap of differentially regulated genes in old versus young compared to progeria versus young cells (FIG. 3B). Finally, Gene Ontology analysis of this data shows enrichment of clearly distinct signaling pathways in healthy old or progeria cells compared to young samples (FIG. 3C). Taken together, these data indicate that separate pathways might underlie the common phenotypes of premature and real aging and highlight the necessity of finding alternative strategies to induce age, with higher fidelity to the natural aging process.

2a. Novel Strategy for Induced In-Vitro Aging Through Moderate Genomic DNA Hypomethylation

In light of recent results described above in section 2, an improved in-vitro aging paradigm is described to better reflect the natural aging process. Here, a strategy is proposed based on inducing moderate, genome-wide DNA hypomethylation, to recapitulate a physiological aging mechanism that is evolutionarily conserved across species and tissues.

Gradual loss of genome-wide DNA methylation with age is the most robust molecular signature of aging from vertebrates to mammals including humans. Global hypomethylation is a molecular hallmark of both aged and cancerous cells^{1,2,11,12}. In-vivo, methylation loss correlates with several age-related diseases in addition to cancer, in particular neurodegenerative disorders^{12,26}. While it has been shown that genomic hypomethylation can cause tumor formation^{27,28}, a mechanistic link to aging has not been explored. It was demonstrated that downstream events of DNA hypomethylation lead to genomic instability, giving rise to mutagenic events^{22,23,29}, or cellular senescence^{30,31}. Cancer incidence drastically increases with age, age is the main risk factor for the majority of cancers, and aged and tumor cells share similar genomic features³².

The present example hypothesizes, without being bound to any theory, that below a certain threshold and in absence of mutagenic events, age-related DNA hypomethylation compromises genomic stability and interferes with normal nuclear functions, ranging from transcription to repair, eventually resulting in the loss of homeostasis that defines the aged cellular state.

The effects of genomic DNA hypomethylation have been studied, however mainly in the contexts of embryonic development and cancer. Moreover, these studies mostly involved severe reductions of methylation levels through e.g. knockout mice^{33,34}, strong hypomorphs^{27,28,35} or acute treatments with potent DNA methyltransferase (DNMT) inhibitors³¹. Deletion or drastic reduction of DNMT activity is not compatible with embryonic development in mice or with cellular proliferation in vitro^{27,28,31,33-36}. The effects of a chronic, subtle DNA hypomethylation to levels permissive for cell proliferation, has not been investigated.

This example explores whether inducing a prolonged, moderate decrease in global DNA methylation, such as occurs in aged tissues (−10-30%), is capable of reproducing cellular hallmarks of age. This will be attempted initially by pharmacological means, through weak DNMT inhibitors (Zebularine³⁷) and will involve a “pulse and chase” treatment, aimed at maintaining stable levels of hypomethylation throughout cell cycles. Reinstating DNMT functionality by inhibitor withdrawal is not expected to restore original methylation as the main DNMT activity in somatic cells is mediated by DNMT1, which can maintain methylation levels but has neglectable de novo methylation capacity^{38,39}. In parallel, a genetic approach will be utilized, either by moderate DNMT1 knockdown via siRNA, or through the generation of lines carrying weak DNMT hypomorphic mutations⁴⁰. The ability to promote cellular age with this new strategy will be evaluated first on a phenotypic level, employing the markers described in a recent report′, which have been validated in the lines utilized for the current example. Second, “molecular age” will be measured based on the newly identified aging signatures defined in the genomic screen. The efficacy of this novel age-inducing strategy will be first tested on primary and iPSC-derived fibroblasts. Later, this paradigm will be adapted to disease-relevant cell types such as iPSC-mDA neurons from PD patients.

A decrease in global methylation was confirmed in the cohort of primary cells described by the present example, as well as re-increased levels in their respective iPSC-derived fibroblasts (FIG. 1A). These preliminary data further confirm DNA methylation as a robust aging marker and indicate that re-acquired methylation levels might play a role in the rejuvenating effects of reprogramming.

The present approach proposes that DNA methylation has causative effects for cellular aging, as well as in the previously unexplored attempt to induce low, chronic levels of demethylation, that allow for the accumulation of downstream defects throughout proliferation. The present approach would not only establish a first mechanistic connection between DNA methylation and aging, but would also provide a simple tool to accelerate age in-vitro.

In the present example, to prevent possible secondary mechanisms that could mitigate the effect of hypomethylation, epigenetic derepression can be aided with validated and commercially available histone methyltransferase-inhibitors to prevent compensatory deposition of repressive histone marks such as H9K9me3 by G9A or SUVh1/2. The present example will also attempt to identify specific treatment conditions as well as suitable, sufficiently sensitive assays to detect subtle (−10-30%) decreases in methylation.

2b. Induced Aging by Hypomethylation in iPSC-Derived Lineages for the Modeling of Late-Onset Disease

One embodiment of the present example is to offer an entry point for iPSC technology to the modeling of late-onset diseases. It has recently been shown that ectopic expression of progerin triggers the appearance of age dependent phenotypes of PD, unseen in previous PD iPSC-models^{7,41}. The current example aims at refining the technique towards a more physiological, non-pathological way to accelerate age in-vitro.
The present example tests whether the age-inducing paradigm described herein allows for improved modeling of the age dependent effects of PD in iPSC-derived mDA neurons from PD patients. The methodology optimized above will be transferred into iPSC-mDA, which could require cell-type specific adaptations to the protocol. Initially, a detailed in-vitro and in-vivo phenotypic comparison will be performed of PD-iPSC-derived mDA neurons that have been aged with the novel protocol described herein, or, in comparison, with the established progerin method, focusing on a set of known features previously described for PD-derived mDA neurons⁷. Finally, the in-vivo significance of the new approach for promoting “true biological age” will be evaluated by aligning the gene expression profiles of in-vitro aged iPSC-mDA neurons to primary human brain tissue from substantia nigra of old and young donors. Primary tissue is available from the National Disease Research Interchange (NDRI), through which a set of samples of different ages has been collected that will soon enter the pipeline for gene-expression and DNA methylation profiling.
The present example is focused on iPSC-based models of PD, however, given that age-associated DNA hypomethylation was reported for most tissues, the methodology described herein could be applied to other cell types, inside and outside of the central nervous system. In this context, the approach described herein may also be applied to iPSC-models of e.g. Alzheimer's disease (AD) or Amyotrophic Lateral Sclerosis (ALS).

In certain embodiments, the strategy described herein is based on inhibition of DNMTs, whose activity in adult somatic tissues mainly consists in the maintenance of methylation patterns in a replication-dependent fashion. Neurons do not exhibit replication-dependent DNMT activity. As such, in certain embodiments, hypomethylation will be induced during the patterning stage of the neural induction protocol⁴². This stage is transient and equivalent to a neural stem cell (NSC) population. In addition to the transient NSC population, a highly neurogenic long-term neural stem cell line (LTNSC) is available that can be patterned to differentiate into various neurons including mDA and on which various demethylation strategies can be tested that will give us the strongest demethylation without altering cell fate.

### Methods

1. Primary fibroblasts from healthy and HGPS donors were obtained from Coriell and comprise 4 lines per donor group (young: 10-11 y, middle-aged: 31-51 y, old: 71-96 y and HGPS: 3-14 y). Sequencing is conducted at the Weill Cornell (WCMC) Epigenomics Core. RNA-Seq is based on both PolyA± (FIG. 1) and Total RNA-Seq (in progress). DNA methylation was profiled by Enhanced Reduced Representation Bisulphite Sequencing (ERRBS). A platform for customized methylation analysis (“clock-CpGs”¹⁴, Aim1) was developed using the Sure Select system (Agilent). 5hmC analysis is performed according to reference (43). ChIP-Seq of histone modifications comprises H3K9me3, H3K27me3, H4K4me3, and H3K36me3. Chromatin preparation conditions have been optimized based on the Covaris truChIP protocol. Generation of iPSCs was done by SeV reprogramming. iPSC validation is based on pluripotency markers, karyotyping, STR profiling (FIG. 2) and EB formation. iPSC-fibroblasts are derived according to reference (7). A different protocol may be used for the directed differentiation of iPSC into a paraxial dermatome fate, the specific lineage that gives rise to dermal fibroblasts.

The present example aims to elicit moderate demethylation, to a slightly higher degree than what observed in-vivo (−10% to −30% of young levels), to achieve an accelerated aging effect. Cells may be exposed to “pulse-chase” treatments and/or low concentrations of weak DNMT inhibitors (Zebularine³⁷) and/or moderate DNMT1 knockdown via Dox-inducible siRNA, allowing for titratable siRNA dosage. To eliminate off target effects inducible lines carrying weak DNMT1 hypomorphic mutations may be generated (e.g. as described by reference 40). Preliminary experiments may be used to determine suitable conditions (concentration and duration) of chemical or siRNA treatment, to attain the desired methylation levels and elicit aging phenotypes. Screens may be conducted using a high-content imaging system (Operetta) that allows for automated image acquisition and analysis in a multi-well format. Experiments may initially be carried out in primary young fibroblasts and iPSC-derived fibroblasts, compared to primary old and iPSC-fibroblasts aged with progerin. This paradigm may subsequently be transferred to iPSC-derived, disease-relevant cell types such as iPSC-mDA neurons from PD patients.

Midbrain dopamine neurons (mDA) for in-vitro and in-vivo induced aging may be generated from PD-iPSC by developed protocols⁴². A broad range of genetic PD-iPSC lines is available through participation in the PD-iPSC consortium (http://pdips.org). To assess the impact of induced aging on in-vitro modeling of PD in iPSC-mDAs, focus may be on three main phenotypes: neurite degeneration, apoptosis and α-synuclein aggregation. In-vivo analysis will involve transplantation studies into 6-OH-DA lesioned NODSCID mice.

### Discussion

Effective disease-modeling via iPSC depends upon the generation of relevant cellular phenotypes. A limitation of the iPSC system is the incapability of reproducing typical degenerative aspects of age-dependent disease. Evidence has been recently provided that this obstacle is to be attributed to a rejuvenating effect of the reprogramming process, which generates cells that are too young to display age-dependent phenotypes. Accordingly, it is widely accepted that differentiation of iPSC into various lineages yields fetal-like cells. These findings open new avenues for the interrogation of the nature of aging and its programming at the cellular level. Yet, they also raise the question as to how well iPSC-technology can model age-dependent conditions. One argument is that most iPSC work done to date utilizes cells that are not sufficiently aged to exhibit degenerative phenotypes of late-onset diseases. To circumvent this barrier, current approaches seek to elicit pathological cell responses with toxic compounds. While environmental factors are major risk factors for disease, acute exposure does not recapitulate the natural progression of disease and hence, results obtained by these means are of questionable relevance. The present example proposes that faithful modeling of late-onset conditions with iPSC requires the incorporation of biological age. A recent study utilizing progerin to accelerate age in-vitro provides proof-of-principle that induced aging of iPSC-derived lineages is necessary and feasible to attain relevant in-vitro models of diseases of age. However, in light of recent data, indicating a discrepancy between progeria and real age, there is a need of devising alternative strategies with closer resemblance to the physiological aging process.

The methods described herein have the dual aim to uncover the molecular mechanisms that dictate rejuvenation through pluripotency and at the same time present a novel approach to induce age in-vitro by mimicking a naturally occurring process, namely the gradual loss of genomic DNA methylation with age.

Induced pluripotent stem cells (iPSC) are a powerful technology for the study of human disease. However, while the study of developmental and juvenile disorders through iPSC has yielded consistent results in reproducing pathological mechanisms in-vitro, modeling of late-onset conditions, such as Parkinson's (PD) or Alzheimer's disease (AD), is still limited by difficulties in recreating characteristic degenerative phenotypes. The lack of age-dependent phenotypes in iPSC-derived cells may be due to their immature and youthful nature, and thus, that effective modeling of neurodegenerative and other age-dependent disorders requires the implementation of cellular “age”. This is supported by recent work, which describes how phenotypic age marks are erased upon reprogramming and not re-acquired after differentiation. Furthermore, in-vitro acceleration of cellular age using progerin, a mutant protein responsible for premature aging, was sufficient to elicit previously unseen, degenerative features in iPSC-derived dopamine neurons from PD patients.

While this approach provided proof of concept for the application of induced aging to iPSC disease models, data from the present example shows that progeroid aging and normal aging are considerably distinct on a transcriptional and epigenetic level. Expression of progerin might therefore not fully recapitulate real aging and potentially compromise experimental results.

The overarching aim of this example is to enhance current iPSC-based disease models by developing improved strategies to induce physiological age, a fundamental component of neurodegenerative pathologies such as PD and AD.

First, the present example aims to elucidate how cellular age is reset upon reprogramming on a genomic level, through a comprehensive analysis of the transcriptional and epigenetic changes prior to and after reprogramming.

### REFERENCES

1. Heyn, H., et al. Distinct DNA methylomes of newborns and centenarians. Proc Natl Acad Sci USA 109, 10522-10527 (2012).
2. Lopez-Otin, C., Blasco, M. A., Partridge, L., Serrano, M. & Kroemer, G. The hallmarks of aging. Cell 153, 1194-1217 (2013).
3. Rando, T. A. & Chang, H. Y. Aging, rejuvenation, and epigenetic reprogramming: resetting the aging clock. Cell 148, 46-57 (2012).
4. Mahmoudi, S. & Brunet, A. Aging and reprogramming: a two-way street. Curr Opin Cell Biol 24, 744-756 (2012).
5. Freije, J. M. & Lopez-Otin, C. Reprogramming aging and progeria. Curr Opin Cell Biol 24, 757-764 (2012).
6. Marion, R. M., et al. Telomeres acquire embryonic stem cell characteristics in induced pluripotent stem cells. Cell stem cell 4, 141-154 (2009).
7. Miller, J. D., et al. Human iPSC-Based Modeling of Late-Onset Disease via Progerin-Induced Aging. Cell Stem Cell 13, 691-705 (2013).
8. Lapasset, L., et al. Rejuvenating senescent and centenarian human cells by reprogramming through the pluripotent state. Genes Dev 25, 2248-2253 (2011).
9. Agarwal, S., et al. Telomere elongation in induced pluripotent stem cells from dyskeratosis congenita patients. Nature 464, 292-296 (2010).
10. Frobel, J., et al. Epigenetic rejuvenation of mesenchymal stromal cells derived from induced pluripotent stem cells. Stem cell reports 3, 414-422 (2014).
11. Fraga, M. F. & Esteller, M. Epigenetics and aging: the targets and the marks. Trends in genetics: TIG 23, 413-418 (2007).
12. Johnson, A. A., et al. The role of DNA methylation in aging, rejuvenation, and age-related disease. Rejuvenation research 15, 483-494 (2012).
13. Bocklandt, S., et al. Epigenetic predictor of age. PloS one 6, e14821 (2011).
14. Horvath, S. DNA methylation age of human tissues and cell types. Genome Biol 14, R115 (2013).
15. Weidner, C. I., et al. Aging of blood can be tracked by DNA methylation changes at just three CpG sites. Genome Biol 15, R24 (2014).
16. Florath, I., Butterbach, K., Muller, H., Bewerunge-Hudler, M. & Brenner, H. Cross-sectional and longitudinal changes in DNA methylation with age: an epigenome-wide analysis revealing over 60 novel age-associated CpG sites. Human molecular genetics 23, 1186-1201 (2014).
17. Hua, H., et al. iPSC-derived beta cells model diabetes due to glucokinase deficiency. The Journal of clinical investigation 123, 3146-3153 (2013).
18. Lee, G., et al. Modelling pathogenesis and treatment of familial dysautonomia using patient-specific iPSCs. Nature 461, 402-406 (2009).
19. Lafaille, F. G., et al. Impaired intrinsic immunity to HSV-1 in human iPSC derived TLR3-deficient CNS cells. Nature 491, 769-773 (2012).
20. Lee, G. & Studer, L. Induced pluripotent stem cell technology for the study of human disease. Nat Methods 7, 25-27 (2010).
21. Nguyen, H. N., et al. LRRK2 mutant iPSC-derived DA neurons demonstrate increased susceptibility to oxidative stress. Cell stem cell 8, 267-280 (2011).
22. Seibler, P., et al. Mitochondrial Parkin recruitment is impaired in neurons derived from mutant PINK1 induced pluripotent stem cells. J Neurosci 31, 5970-5976 (2011).
23. Woodard, C. M., et al. iPSC-Derived Dopamine Neurons Reveal Differences between Monozygotic Twins Discordant for Parkinson's Disease. Cell reports 9, 1173-1182 (2014).
24. Eriksson, M., et al. Recurrent de novo point mutations in lamin A cause Hutchinson-Gilford progeria syndrome. Nature 423, 293-298 (2003).
25. Burtner, C. R. & Kennedy, B. K. Progeria syndromes and ageing: what is the connection? Nature reviews. Molecular cell biology 11, 567-578 (2010).
26. Lu, H., Liu, X., Deng, Y. & Qing, H. DNA methylation, a hand behind neurodegenerative diseases. Frontiers in aging neuroscience 5, 85 (2013).
27. Eden, A., Gaudet, F., Waghmare, A. & Jaenisch, R. Chromosomal instability and tumors promoted by DNA hypomethylation. Science 300, 455 (2003).
28. Gaudet, F., et al. Induction of tumors in mice by genomic hypomethylation. Science 300, 489-492 (2003).
29. Rodriguez, J., et al. Chromosomal instability correlates with genome-wide DNA demethylation in human primary colorectal cancers. Cancer research 66, 8462-9468 (2006).
30. Holliday, R. Strong effects of 5-azacytidine on the in vitro lifespan of human diploid fibroblasts. Experimental cell research 166, 543-552 (1986).
31. Young, J. I. & Smith, J. R. DNA methyltransferase inhibition in normal human fibroblasts induces a p21-dependent cell cycle withdrawal. The Journal of biological chemistry 276, 19610-19616 (2001).
32. Zane, L., Sharma, V. & Misteli, T. Common features of chromatin in aging and cancer: cause or coincidence? Trends in cell biology 24, 686-694 (2014).
33. Li, E., Bestor, T. H. & Jaenisch, R. Targeted mutation of the DNA methyltransferase gene results in embryonic lethality. Cell 69, 915-926 (1992).
34. Okano, M., Bell, D. W., Haber, D. A. & Li, E. DNA methyltransferases Dnmt3a and Dnmt3b are essential for de novo methylation and mammalian development. Cell 99, 247-257 (1999).
35. Lei, H., et al. De novo DNA cytosine methyltransferase activities in mouse embryonic stem cells. Development 122, 3195-3205 (1996).
36. Yamada, Y., et al. Opposing effects of DNA hypomethylation on intestinal and liver carcinogenesis. Proc Natl Acad Sci USA 102, 13580-13585 (2005).
37. Kilgore, J. A., et al. Identification of DNMT1 selective antagonists using a novel scintillation proximity assay. The Journal of biological chemistry 288, 19673-19684 (2013).
38. Chuang, L. S., et al. Human DNA-(cytosine-5) methyltransferase-PCNA complex as a target for p21WAF1. Science 277, 1996-2000 (1997).
39. Bostick, M., et al. UHRF1 plays a role in maintaining DNA methylation in mammalian cells. Science 317, 1760-1764 (2007).
40. Egger, G., et al. Identification of DNMT1 (DNA methyltransferase 1) hypomorphs in somatic knockouts suggests an essential role for DNMT1 in cell survival. Proc Natl Acad Sci USA 103, 14080-14085 (2006).
41. Koch, P., Opitz, T., Steinbeck, J. A., Ladewig, J. & Brustle, 0. A rosette-type, self-renewing human ES cell-derived neural stem cell with potential for in vitro instruction and synaptic integration. Proc Natl Acad Sci USA 106, 3225-3230 (2009).
42. Kriks, S., et al. Dopamine neurons derived from human ES cells efficiently engraft in animal models of Parkinson's disease. Nature 480, 547-551 (2011).
43. Song, C. X., et al. Selective chemical labeling reveals the genome-wide distribution of 5-hydroxymethylcytosine. Nat Biotechnol 29, 68-72 (2011).

### Example 11

### Reducing Age in Cells by Increasing Methylation

### Introduction

PIWI proteins are known as the germline-specific clade of the Argonaute superfamily of RNAi effector proteins. PIWIs are expressed in all metazoans analyzed so far and through the interaction with their RNA partners, piRNAs (PIWI-interacting RNAs), they suppress the activity of transposable elements in germ cells, an essential function for germline development and fertility (Aravin et al., 2007, Juliano et al., 2011). Silencing of transposons and other repetitive sequences (such as centromeric and telomeric regions) by the PIWI-piRNA system employs multiple pathways. The best-characterized mechanism is a direct cleavage of repeat transcripts through the endonucleolytic activity of PIWI proteins. In addition, transcriptional repression can occur via epigenetic means, i.e. the recruitment of heterochromatin-forming factors, including histone modifiers and DNA methyltransferases (DNMTs) (Peng and Lin 2013). It was shown that PIWI proteins mediate de novo DNA methylation of transposable elements in the mammalian germline (Kuramochi-Miyagawa et al., 2008). Locus-specificity of PIWI-mediated silencing is imparted by the interaction with a piRNA guide, which directs PIWIs to specific genomic regions through complementary base-pairing with target sequences. Here, PIWI-piRNA complexes recruit a multitude of epigenetic modifiers that initiate transcriptional repression and heterochromatinization of the target locus (Ross et al., 2014).

In addition to an involvement in metazoan germline development and fertility, PIWI proteins have recently been implicated in organismal longevity in C. elegans (Simon et al., 2013). PIWI proteins are also expressed in the adult stem cell niche throughout evolution, including in humans, where they are found e.g. in hematopoietic stem cells and progenitors. While immortality of the germline is believed to depend upon the ability to safeguard genomic integrity over generations, e.g. by keeping parasitic elements in check, PIWI-mediated genome protection may provide a fundamental mechanism to preserve multipotency and self-renewal of adult stem cells throughout organismal lifespan (reviewed in Juliano et al., 2011, Ross et al., 2014).

### Methods

A large portion of the age-related nuclear defects mediated by a loss of global DNA methylation and repressive histone marks may be attributed to a hyperactivation of transposable elements and other repetitive sequences. Increased expression of genomic repeats in aged tissues has been previously described (Heyn et al., 2012).

The present example describes a technique that employs the PIWI-piRNA system to restore epigenetic silencing at those loci that are aberrantly expressed in aged tissues as a consequence of DNA hypomethylation and loss of repressive histone marks. PIWI proteins are enriched in the germline and adult stem cell compartments, and are absent from most somatic tissues. Controlled re-introduction of PIWI proteins in somatic cells, in concert with targeted, locus specific, piRNA expression, could represent a strategy to direct re-silencing of repetitive and parasitic genomic loci that have aberrantly lost epigenetic repression as a function of chronological age.

Results

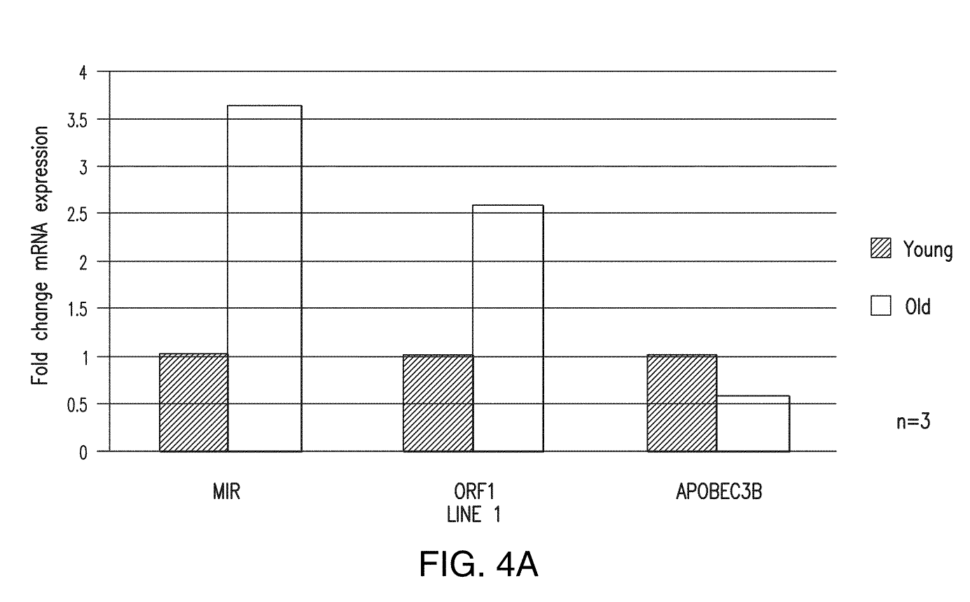

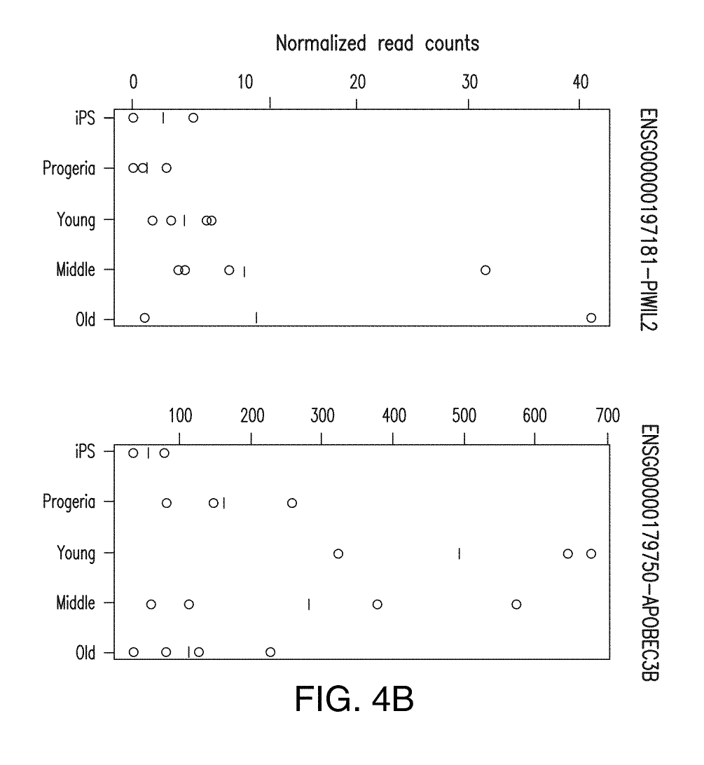

The presence of LINE1 and MIR elements in primary fibroblasts of young and old donors was determined using RT-qPCR. Increased expression of the analyzed repetitive elements was detected in old samples compared to young samples (FIG. 4A).

PIWIL2 and APOBEC3B expression in primary fibroblasts from different age donor groups was determined using RNA-Seq analysis. Minimal expression of PIWI proteins was detected in somatic cells. Additionally, a gradual decrease in the level of the somatic transposon protection factor APOBEC3B (a somatic factor responsible for transposon clearance) was detected as sample age increased (young>middle-aged>old).

### Example 12

### Fibroblasts from Old Subjects have Decreased Levels of DNA Methylation Methods

Primary fibroblasts were collected from young (aged 10-11 years) and old (aged 71-96 years) subjects. Genome-wide levels of DNA methylation, as well as methylation levels of specific repetitive elements, in the fibroblasts were determined by Reduced Representation Bisulphite Sequencing (ERRBS) as well as by fluorimetric measurement of global DNAm levels. Transcriptional expression of repetitive elements was determined by Total RNA-Seq analysis. Expression levels H3K9me3 and H3K27me3, two marks of transcriptional repression, were also determined using Western blot analysis.

### Results

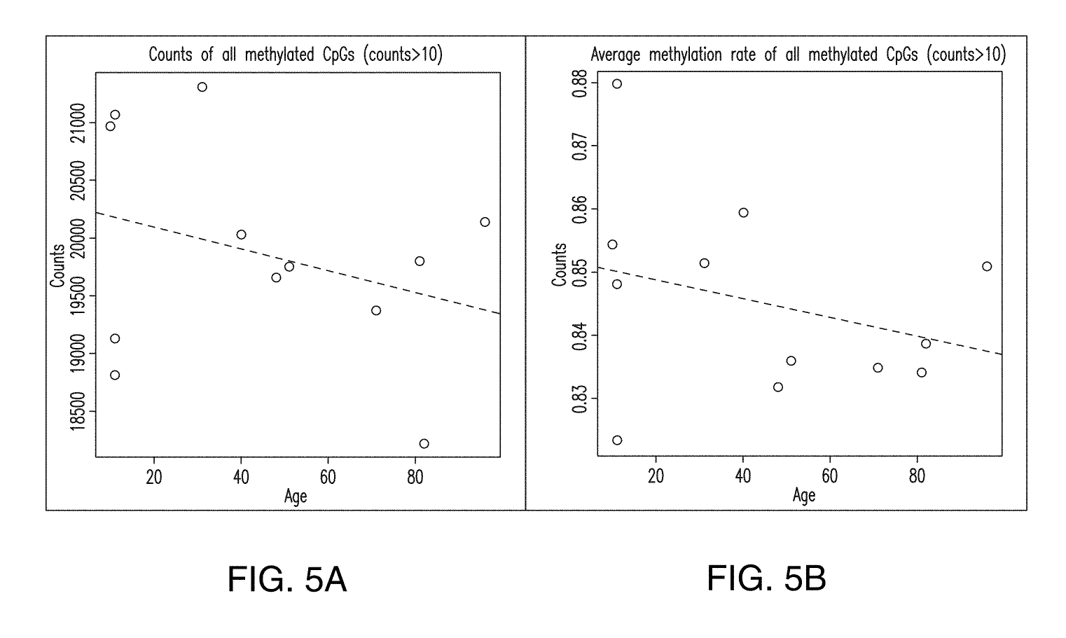

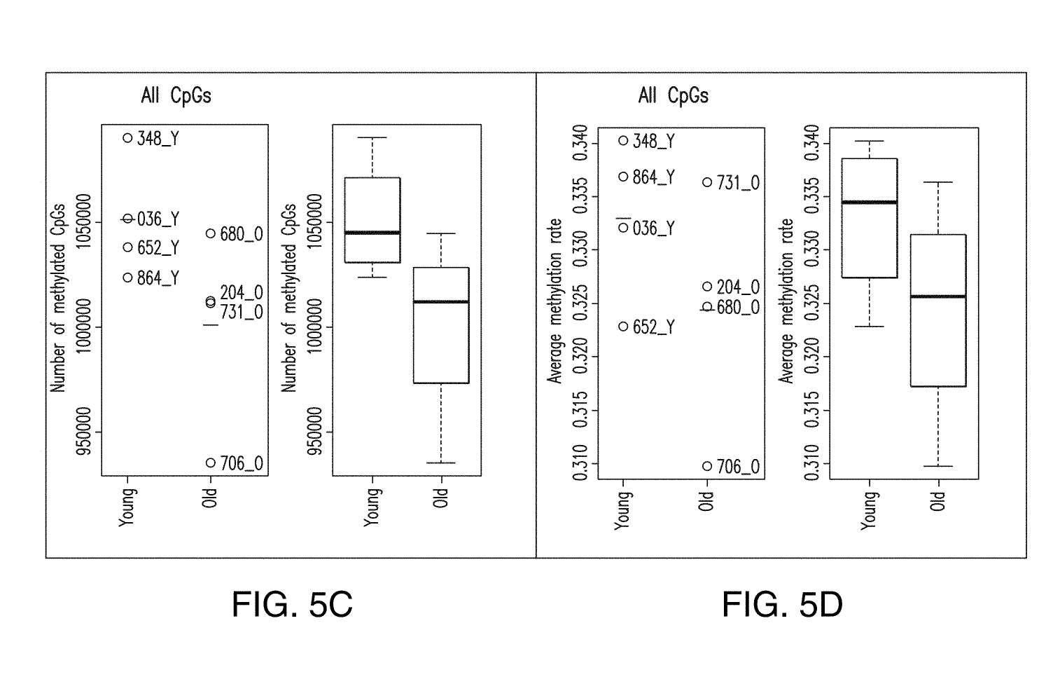

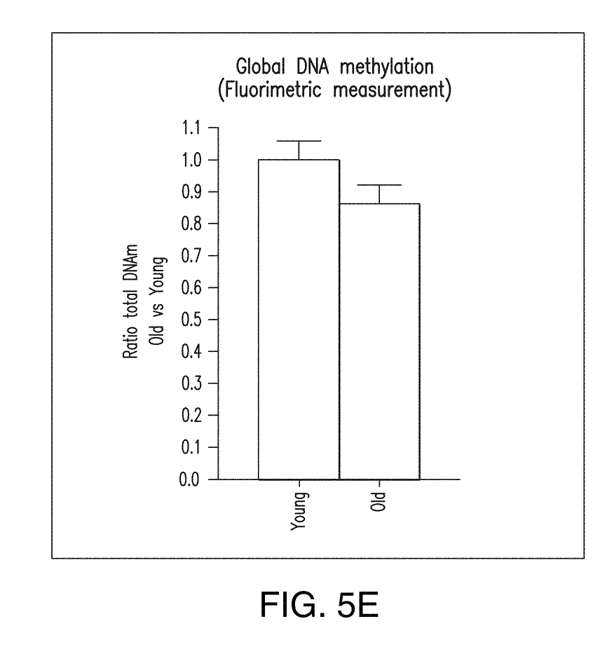

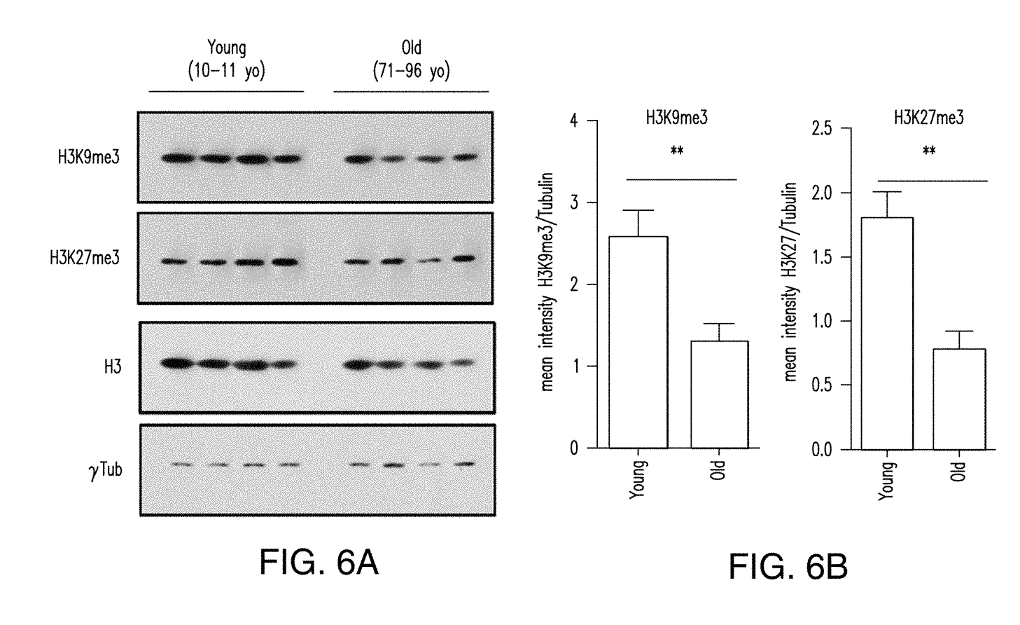

Primary fibroblasts from young subjects exhibited higher levels of global DNAm compared to fibroblasts from old subjects (FIG. 5A-E). The young fibroblasts exhibited a greater number of methylated CpGs (FIGS. 5A and C), as well as a higher rate of CpG methylation (FIGS. 5B and D). Similarly, with regard to the specific epigenetic marks of transcriptional repression, H3K9me3 and H3K27me3, the young fibroblasts exhibited greater expression of these two marks compared to the old fibroblasts (FIG. 6A-B).

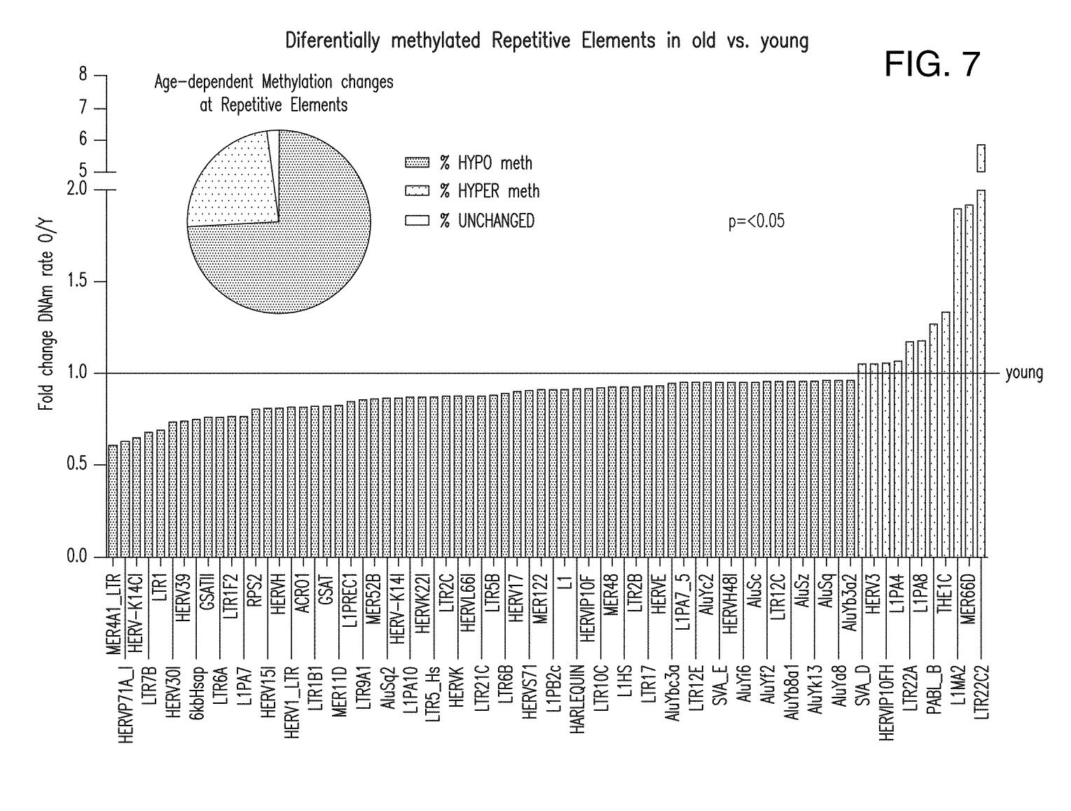

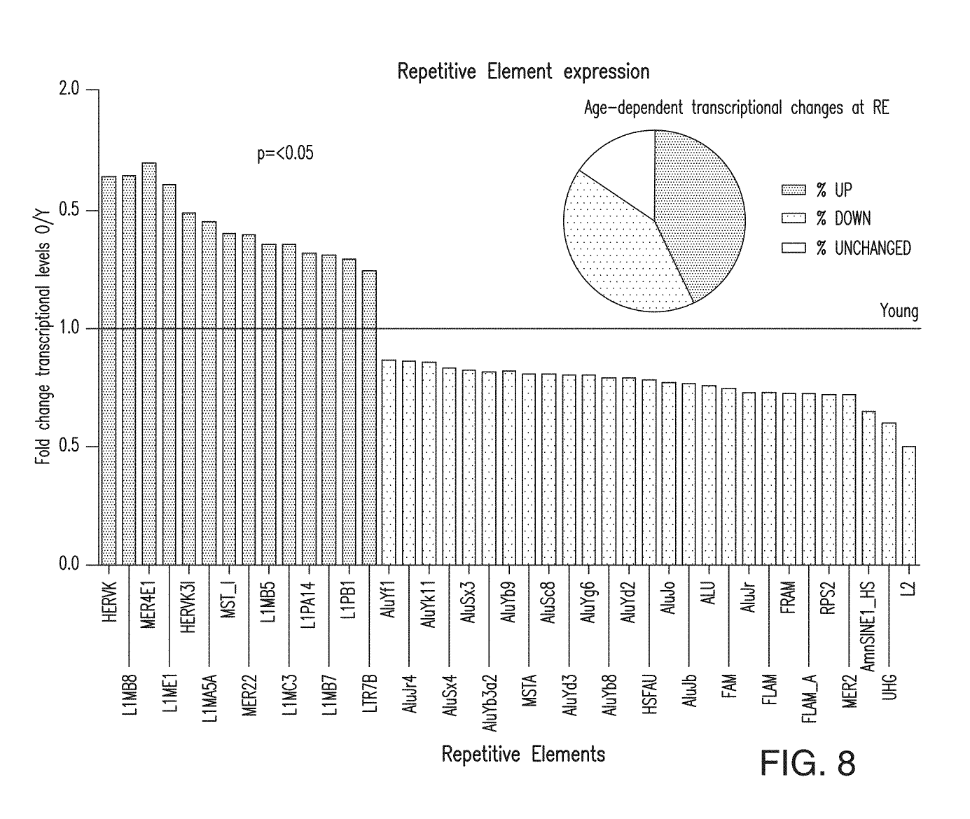

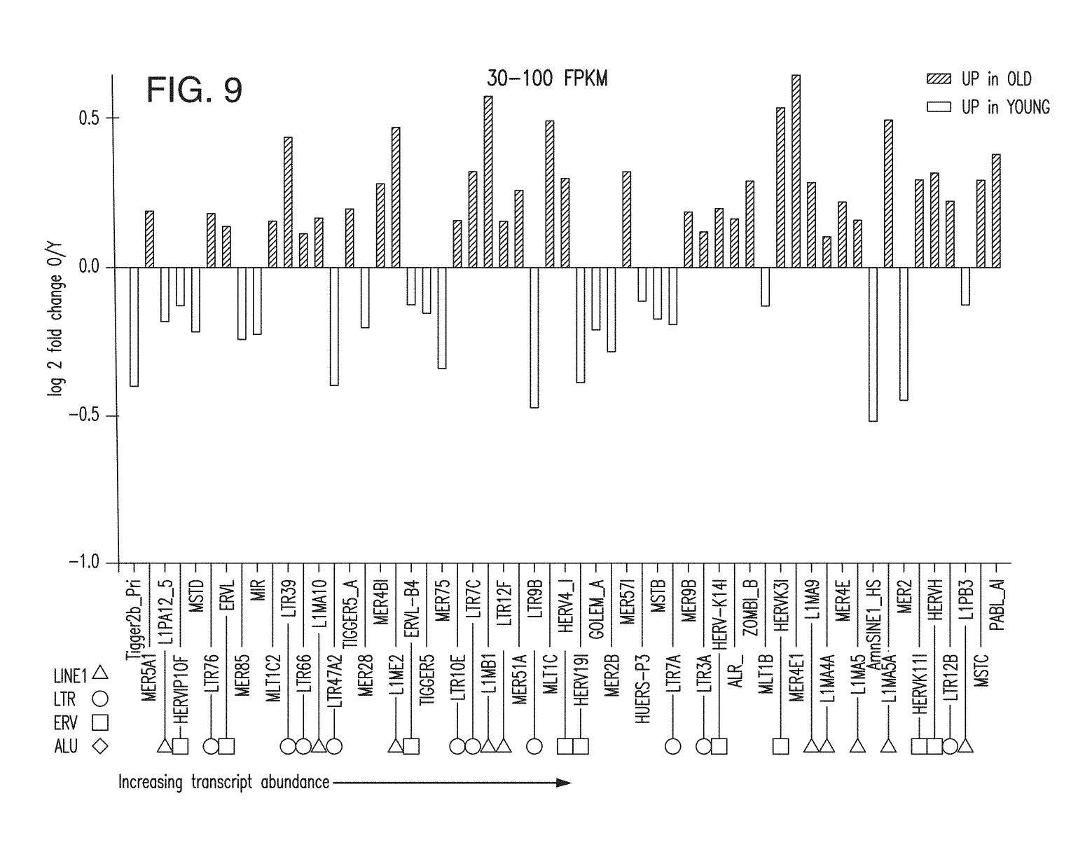

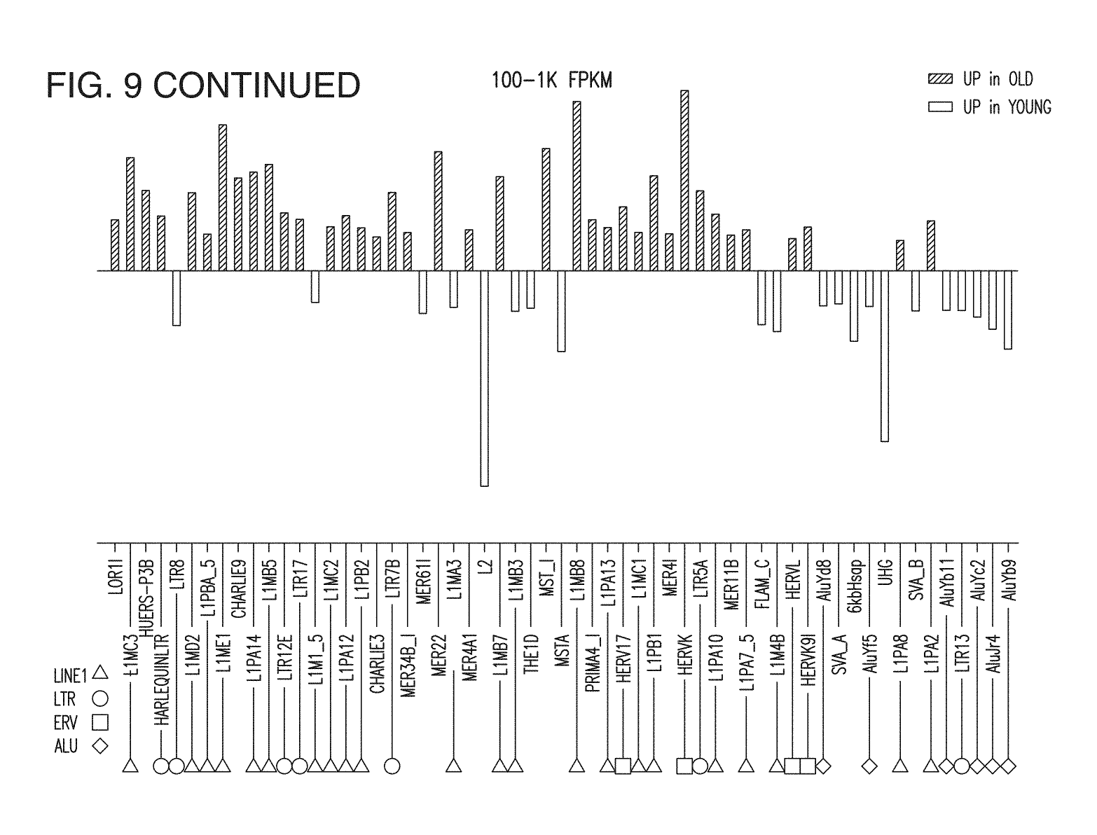

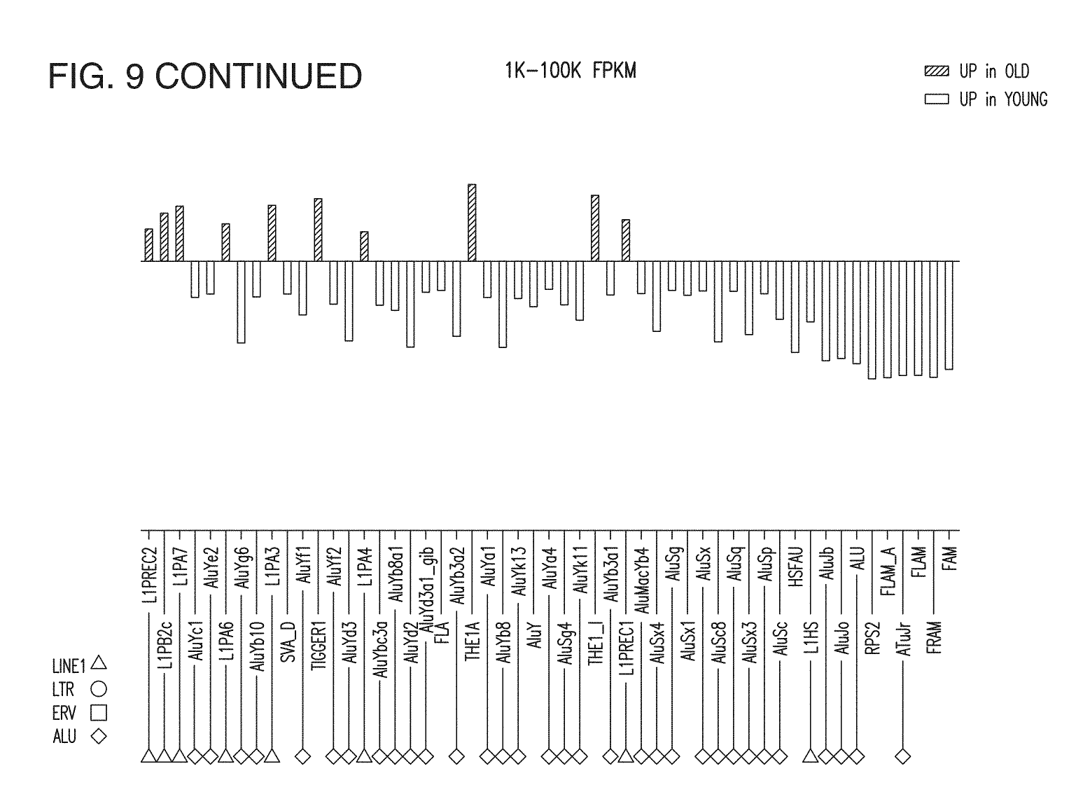

The age-dependent loss of methylation and transcriptional repression was predominant at non-coding repetitive elements, such as transposable elements. (FIGS. 7 and 8), wherein 75% of repetitive elements were hypomethylated in old fibroblasts compared to young fibroblasts (FIG. 7). Additionally, age-dependent differential expression of repetitive transcripts were detected in the old versus young fibroblasts, wherein LINE1 elements were preferentially upregulated and ALU elements downregulated in the old fibroblasts. (FIG. 8). Furthermore, the repetitive elements that were upregulated in the old fibroblasts were primarily low abundance elements (30-1000 FPKM), mainly originating from LINE1 (L1), LTR elements and Endogenous Retroviruses (ERVs), whereas high abundance transcripts (10,000-100,000 FPKM), mostly originating from ALU elements, appear downregulated in the old fibroblasts and upregulated in the young fibroblasts. (FIG. 9).

### Example 13

### Induction of Aged Phenotype in Cells by Increasing DNA Methylation

### Summary

Aging in humans is a process that is not well understood, especially in the realm of late-onset diseases. Induced pluripotent stem cells (iPSCs) provide a promising model to study late-onset disease, but recently it has become clear that inducing pluripotency reverses the age signature of cells, indicating that aging is a “programmed” state. DNA methylation and histone modifications are important processes in epigenetics. DNA methylation occurs through the addition of a methyl group to the 5-carbon of cytosine, creating 5-methylcytosine (5-mC). 5-mC patterns are established during development by DNA methyltransferase (DNMTs), DNMT3a and DNMT3b, and maintained during replication by DNMT1. These 5-mC areas act as inhibitors of transcription by blocking the recruitment of transcription factors, regulating which genes are and are not expressed. To test whether epigenetic modifications could induce either an “aged” or a “rejuvenated” state, young and old fibroblasts were treated with drug compounds that modulated the regulation of DNA methylation and histone modifications. Two of the compounds, Decitabine and Zebularine, act as DNMT1 and DNMT3a&b inhibitors. Another compound, SW155246, acts as a selective DNMT1 inhibitor. The fourth inhibitor compound was Chaetocin which acts as a SUV3/9 inhibitor. SUV3/9 plays an important role in heterochromatin organization and maintenance of histone methylation during cell replication. To determine whether the compounds exhibited an “aged” or “rejuvinating” effect at the cellular level, the level of DNA damage (yH2Ax level), which increases with age, was examined. Expression levels of histone protein H1, heterochromatin marker HP1α, H3K9me3, H3K27me3, nuclear morphology marker LaminB1, and global DNA methylation, which are markers of youthful cellular age, were also examined. The tested compounds were able to modulate the expression levels of the various markers tested.

The effect of resveratrol, a compound in red wine that is a sirtuin 1 (SIRT1) activator, and rapamycin, an mTOR inhibitor, on the epigenetic state of the cells were also determined. Sirtuins are a family of proteins whose function is not well understood; however, studies have shown that SIRT1 plays a role in DNA damage response and metabolism. mTOR is a protein kinase that plays a role in regulating cell proliferation, cell growth, protein synthesis, transcription, and activation of autophagy, a process through which unnecessary or dysfunctional cellular components are degraded, helping cells to survive by maintaining cellular energy levels. Resveratrol and rapamycin increased the levels of the youthful age markers, which was greater in young cells compared to old cells.

### Methods

### Cell Samples

Cell samples were all supplied by Coriell Cell Repositories. The cell line from a young individual (<20 years) was GM03348 (“348”). The cell line from an old individual (>65 years) was GM04204 (“204”).

### Cell Culture

Cells were cultured in human fibroblast medium made from 95% Gibco's Minimum Essential Medium, 4.5% fetal bovine serum, and 0.5% Penicillin/Streptomycin. Cells were plated on 15 cm plates and passaged every 2-3 days as necessary, and fed every other day. For experimentation, cells were plated in 96 well plates or 6 well plates. Before fixation, cells were treated with a preextraction buffer containing 20 mM Hepes pH 7.9, 0.5% Triton X-100, 50 mM NaCl, and 300 mM sucrose for 7 minutes at 4° C. to reduce the high levels of background seen in initial stainings. Cells were then fixed with 4% paraformaldehyde for 15 minutes at room temperature.

### Toxicity Testing

Cells were treated with different concentrations of six compounds for 4 days, then tested for overall cell viability with Resazurin Sodium Salt (Sigma Aldrich). Viable cells are able to reduce resazurin into resofurin, which is highly fluorescent. Cells were placed in medium with 10 ug/ml of resazurin for 90 min, then medium was removed from cells and read through PerkinElmer ENSPIRE plate reader. The plate reader measures fluorescence, which is analyzed based on the positive control for 100% viability, and the negative control for 100% non-viability. The same procedure was repeated again 6 days later for second toxicity timepoint.

### Immunofluorescence Detection of Marker Expression

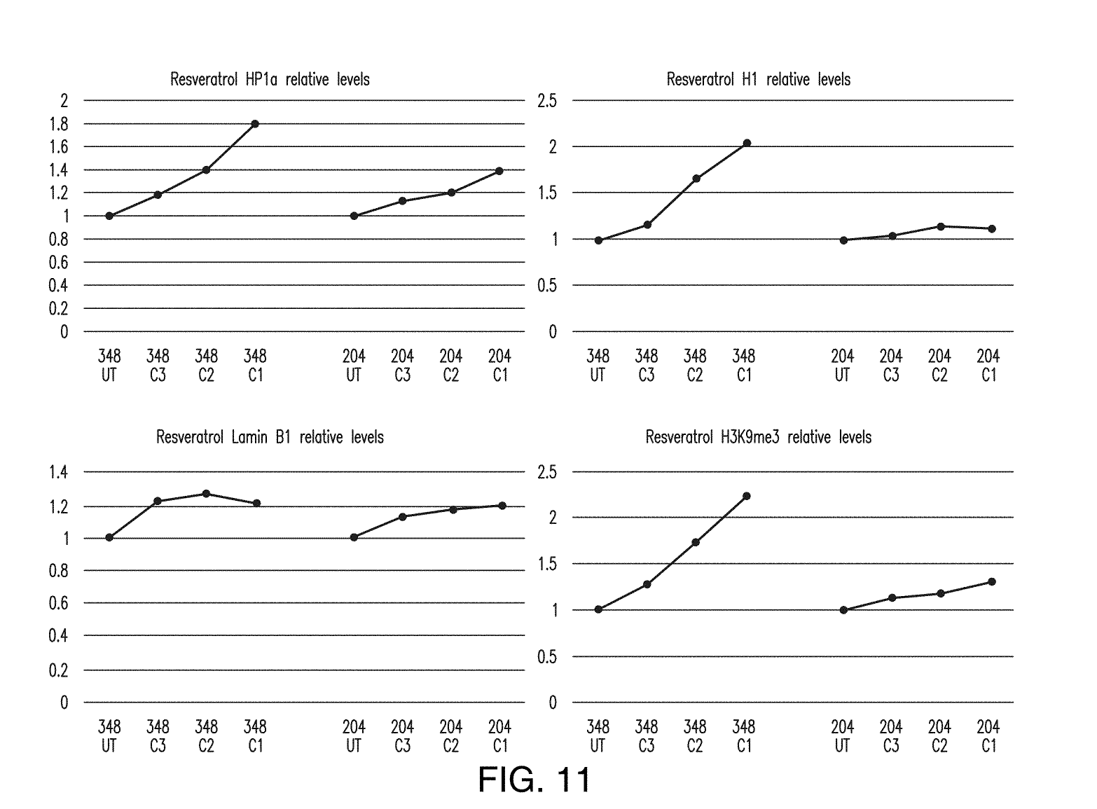

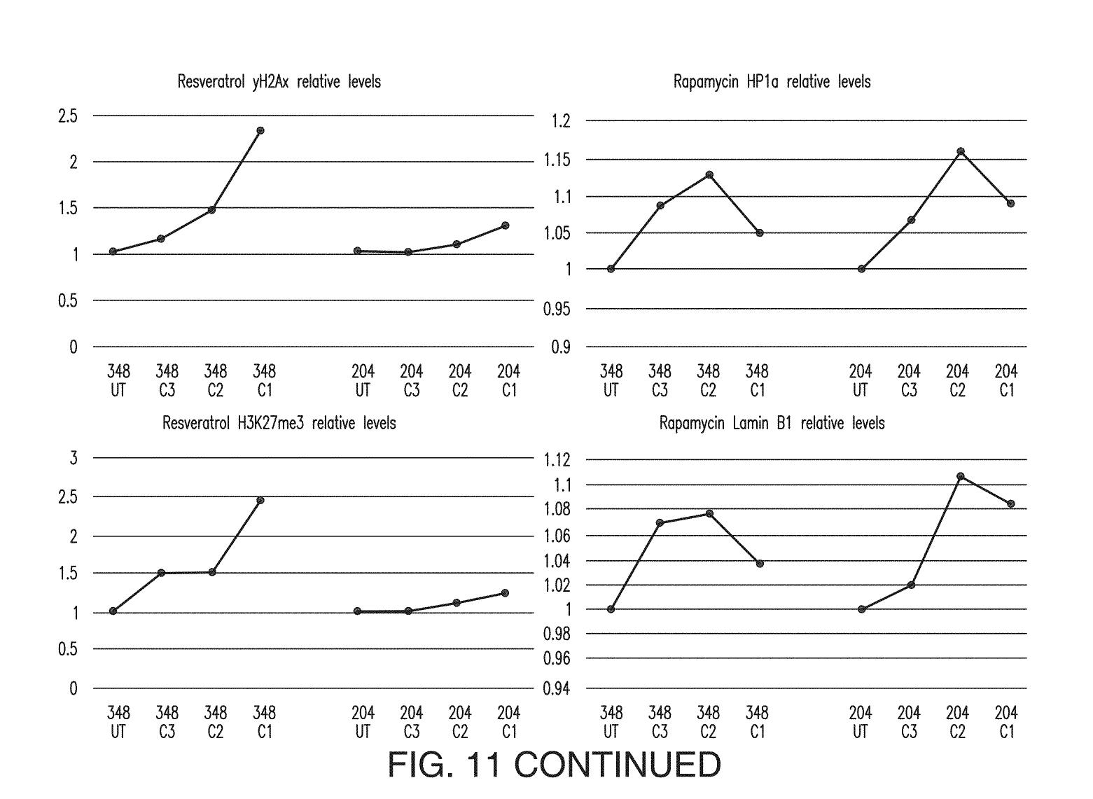

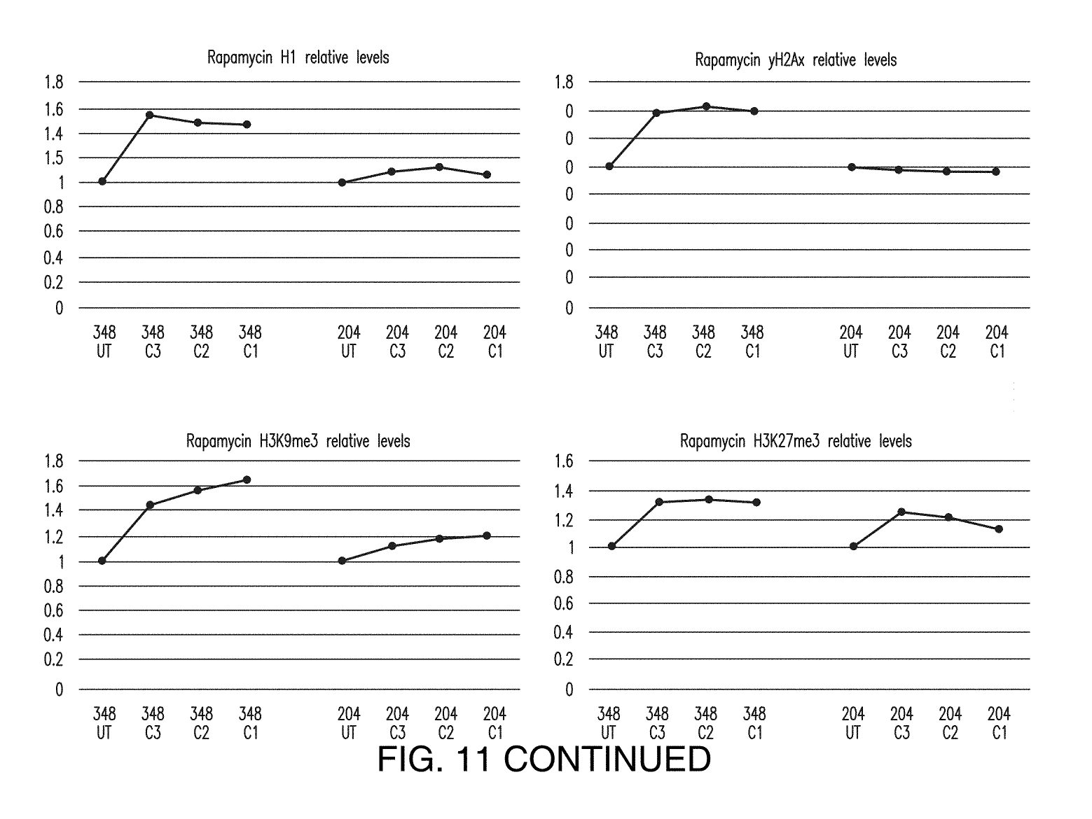

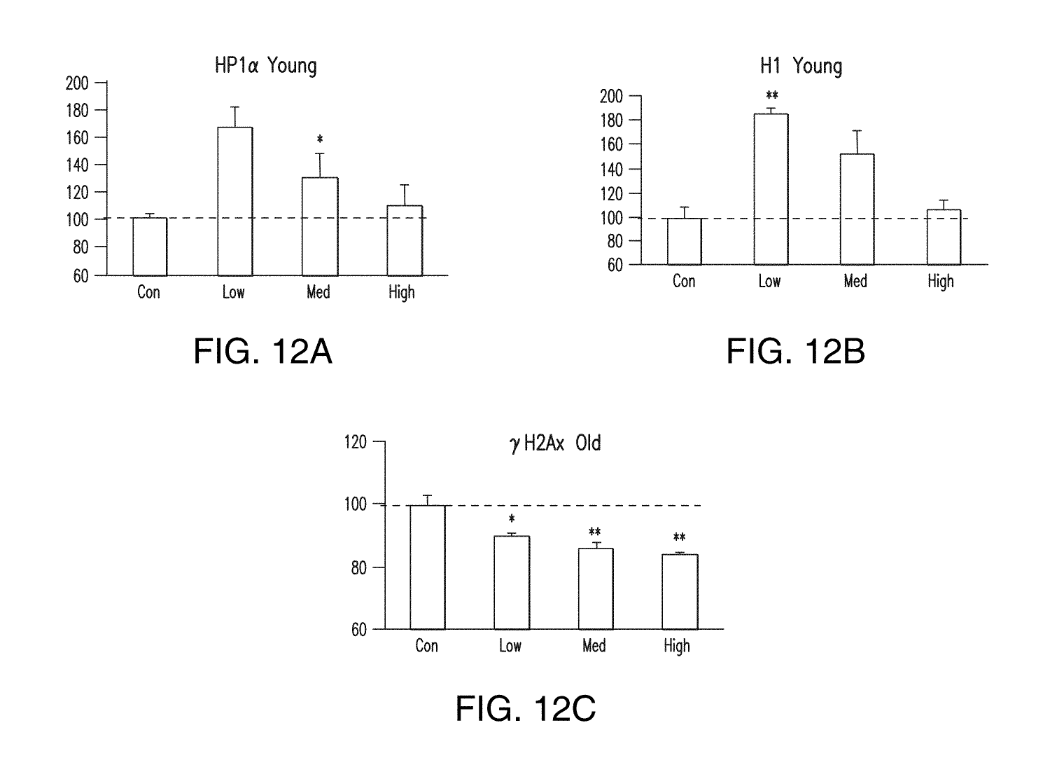

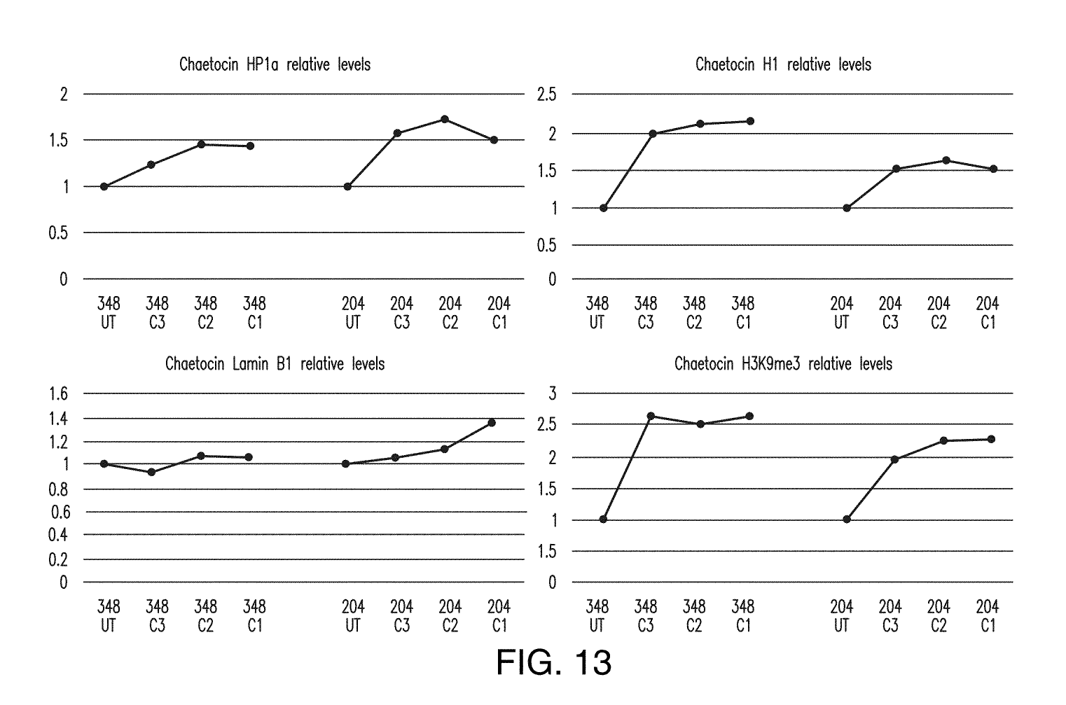

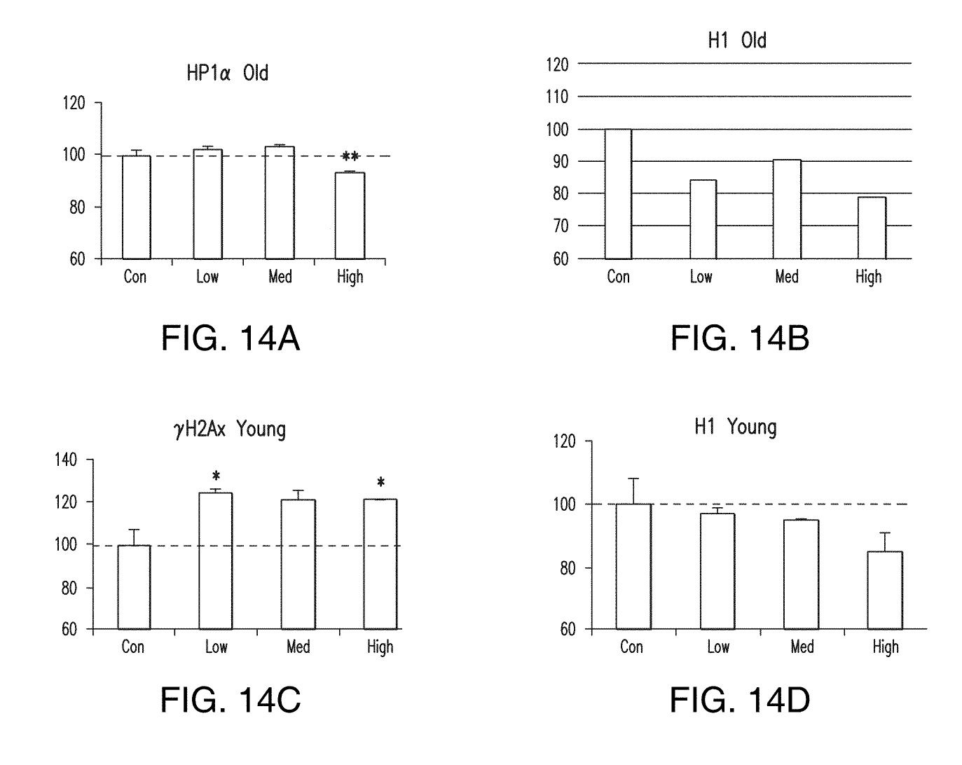

After fixing, cells were incubated in Permeablization Buffer containing 0.3% Triton X-100 and 1% BSA in PBS for 35 minutes at room temperature. Cells were then incubated with primary antibodies to markers (1:1000 in PBS) at 4° C. overnight. Following incubation, cells were washed 3× with PBS, then incubated with secondary antibodies (1:500 in PBS) for 30 minutes at room temperature, protected from light. Following this incubation, cells were washed 1× with PBS, then incubated with DAPI 1:1000 in PBS for 7 minutes at room temperature, protected from light, then washed 2× with PBS. Stainings were imaged using either using the Hamamatsu Olympus IX81 microscope, or the PerkinElmer Operetta High Content Imaging System. The expression level of each marker was determined relative to the untreated control on the same treatment plate (FIGS. 11, 13, 15 and 16), or to the average of the untreated controls across all treatment plates for the same marker (FIGS. 12 and 14).

### Imaging and Quantification

Imaging of immunofluorescence was done using the PerkinElmer Operetta High Content Imaging System. Each well had several (>15) random spots in each plate imaged, and staining intensity quantification was done using the Operetta Harmony software. Analysis is based on mean intensity levels of each staining within a single well compared to mean intensity levels in other wells.

### DNA Methylation Quantification and Analysis

Genomic DNA was extracted from treated cell samples using the Quick-gDNA MicroPrep kit (Zymo). Global levels of DNA methylation were measured using the colorimetric Methylflash Methylated DNA quantification kit (Epigentek) according to manufacturer instructions, and read using the PerkinElmer ENSPIRE plate reader. gDNA sample size was 50 ng. Analysis was performed according to manufacturer instructions.

### Compounds

Stock solutions of each of the compounds were created by resuspending each in DMSO according to the maximum solubility provided by the manufacturer. Stock solutions were: Decitabine (Tocris Bioscience #2293), 50 mM; Zebularine (Tocris Bioscience #2624), 100 mM; SW155246 (Sigma #SML1136), 10 mM; Chaetocin (Tocris Bioscience #4504), 10 mM; Resveratrol (Calbiochem #554325), 100 mM. Rapamycin (Sigma #R8781) came as a 2.74 mM stock solution in DMSO.

### Results

### Determining the Proper Density for Treatments

Experiments were performed with two lines of human dermal fibroblasts, one from a young donor (<18 years old) (“young cells”), and one from an old donor (>65 years old) (“old cells”). Before the cells were to be treated with the compounds, toxicity tests were run to determine what concentration of each compound the cells should be treated with. To determine the proper cell density for this test, we designed an experiment using 96-well plates with 10 different initial cell densities, from 1000 cells/well to 10,000 cells per well, with each density having 6 replicates. After three days, cells were fixed and stained with an antibody to Vimentin (an intermediate filament) and DAPI (a nuclear marker) to determine the ideal cell density. By assessing the confluence of each initial density at the three-day mark, an initial density of 2,500 cells per well was determined to be ideal for the length of the toxicity assay we would be running (7 days).

### Assessment of the Toxicity of Each Drug and Treatment of Cells

The toxicity of each compound was tested before the actual cell treatment was started to determine the maximum concentration that cells could be exposed to without losing viability and the ability to proliferate. Initial concentrations of each drug to be used were determined by examining prior experiments that had used each of the compounds. Cells were treated in 96 well plates, with the first column being a negative control (cells treated with 0.1% Triton-X 100 to ensure cell death), and the last column being a positive control (cells treated with DMSO, the compound used to dissolve the drugs, 1:1000). Triplicates of each intermediate concentration were used, and each concentration was ½ the molarity of the previous concentration. The concentrations of Resveratrol and Zebularine ranged from 400 μM to 0.00038 μM (13, 14). The concentrations of Rapamycin ranged from 100 μM to 0.000095 μM (15). The concentrations of Chaetocin ranged from 30 μM to 0.000028 μM (16). The concentrations of Decitabine ranged from 200 μM to 0.00019 μM (17). The concentrations of SW155246 ranged from 50 μM to 0.00047 μM (18).

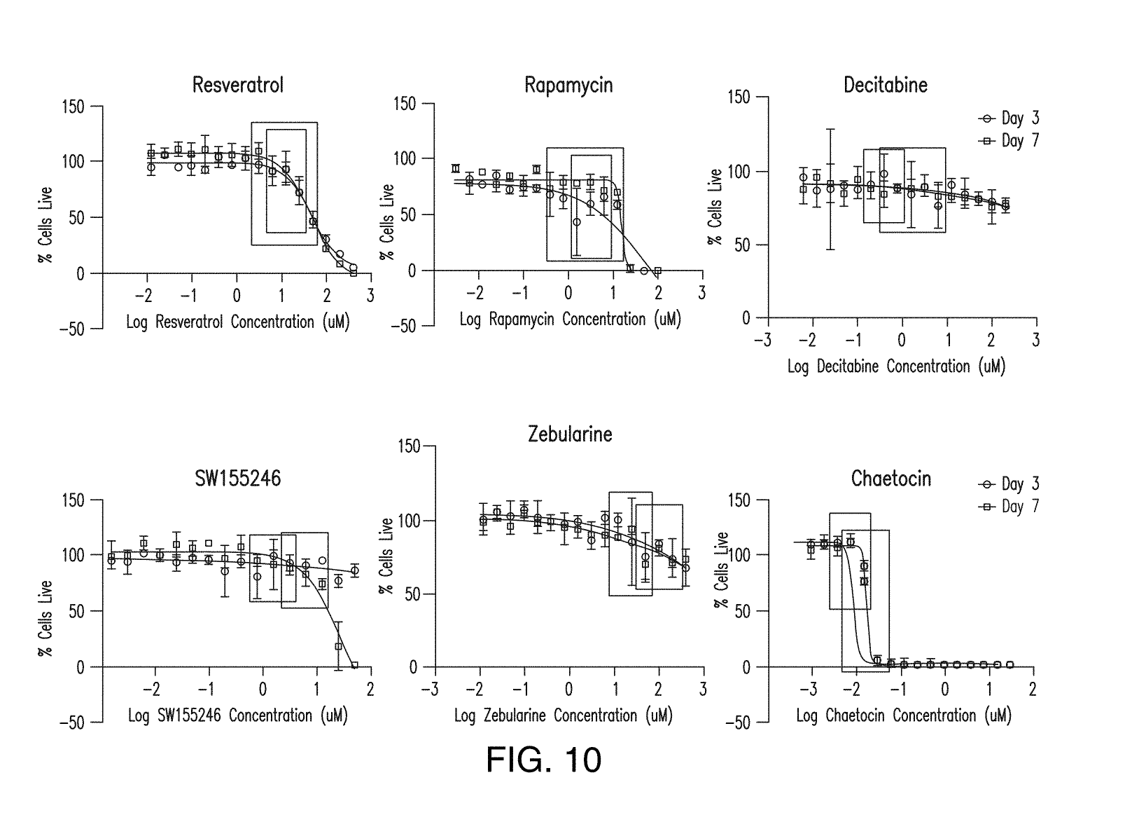

Cells were treated with these concentrations of each compound and tested for toxicity using Resazurin Sodium Salt on Day 4 (see Methods). After the Resazurin assay was conducted, cells were incubated in fresh medium with the same concentrations of compounds as they had been treated with on Day 1, and tested again using the Resazurin assay on Day 7. Using data from both of these days, we determined concentrations of each compound for treatment (FIG. 10). These concentrations are referred to as C1, C2 and C3, with C1 being the highest concentration. The concentrations used were: Resveratrol 25 μM, 12.5 μM and 6.25 μM; Rapamycin 6.25 μM, 3.125 μM, 1.5625 μM; Decitabine 0.8 μM, 0.4 μM, 0.2 μM; Zebularine 50 μM, 25 μM, 12.5 μM; Chaetocin 0.00732 μM, 0.00366 μM, 0.00183 μM; and SW155246 3.2 μM, 1.6 μM, 0.8 μM.

Cells were then treated with each of the six compounds for three days. Each compound had both a young and old untreated control, as well as the three pre-determined concentrations for both young and old cells. In addition, a six-well plate was set up for Decitabine, Zebularine, Chaetocin and SW155246, with an additional 3 cm plate for use as untreated controls for quantification of global levels of DNA methylation. After treatment, cells were stained with immunofluorescence for HP1α, H1, γH2Ax, Lamin B1, H3K9me3 and H2K27me3.

### Effects of Resveratrol and Rapamycin on Aging Markers

After three days of culture with exposure to either Resveratrol or Rapamycin, cells began to show higher levels of “five markers of a younger state,” which are markers that are indicative of a younger cellular “age”. Markers assessed included: histone protein H1, which is a histone linker protein; heterochromatin marker HP1α; H3K9me3 and H3K27me3, both of which are methylated sites correlated with transcriptional repression; and nuclear morphology marker LaminB1. Therefore, aging is associated with a decrease in these markers. DNA damage marker γH2Ax was also examined, where levels of γH2Ax tends to increase with age.

After treatment with Resveratrol, the five markers of a younger state all increased, as shown in FIG. 11 and FIG. 12. Increases in these markers are consistent with an anti-aging effect. However, γH2Ax levels also increased. Resveratrol had more of an effect on young cells than on old cells. Cells treated with Rapamycin also showed an increase in the five markers of a younger state (FIG. 11). There was again a greater effect on young cells than old cells, though the difference between the two ages was less than seen for Resveratrol. Like Rapamycin, Resveratrol also increased γH2Ax. An increase in γH2Ax level, as well as increases in expression of the other markers, can occur at concentratins where the compounds are toxic to the cells.

### Effects of Inhibitor Compounds on Aging Markers

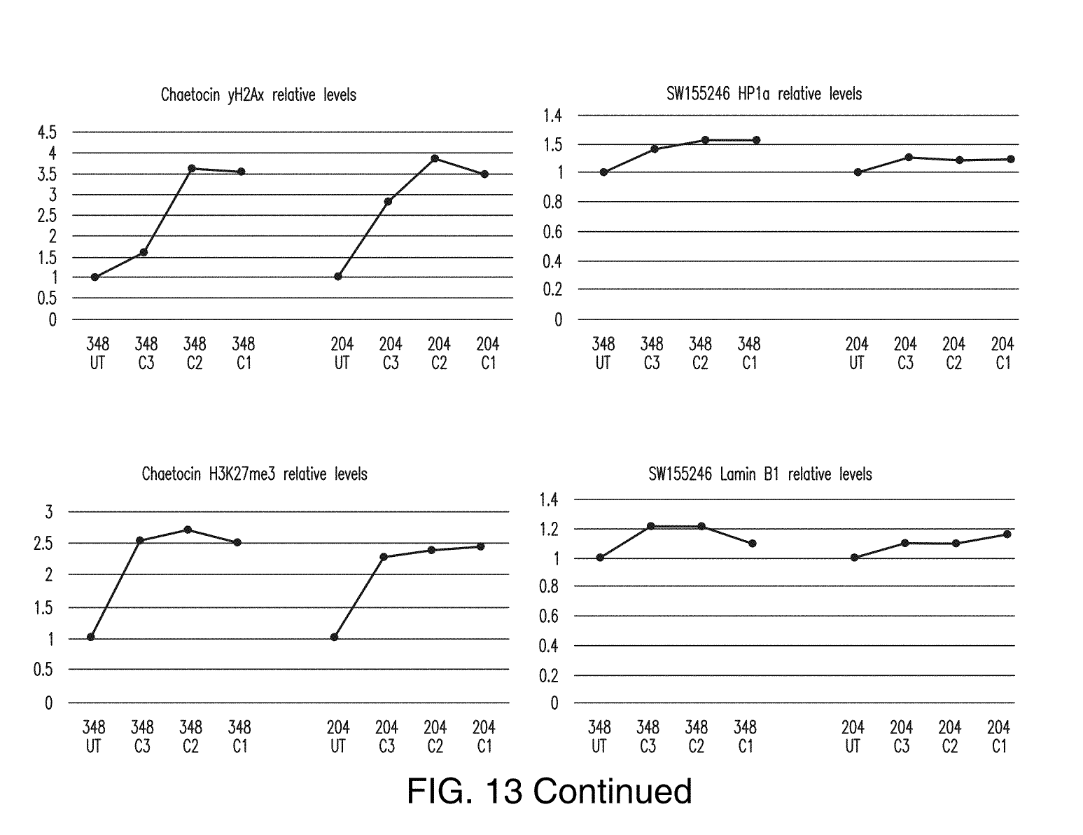

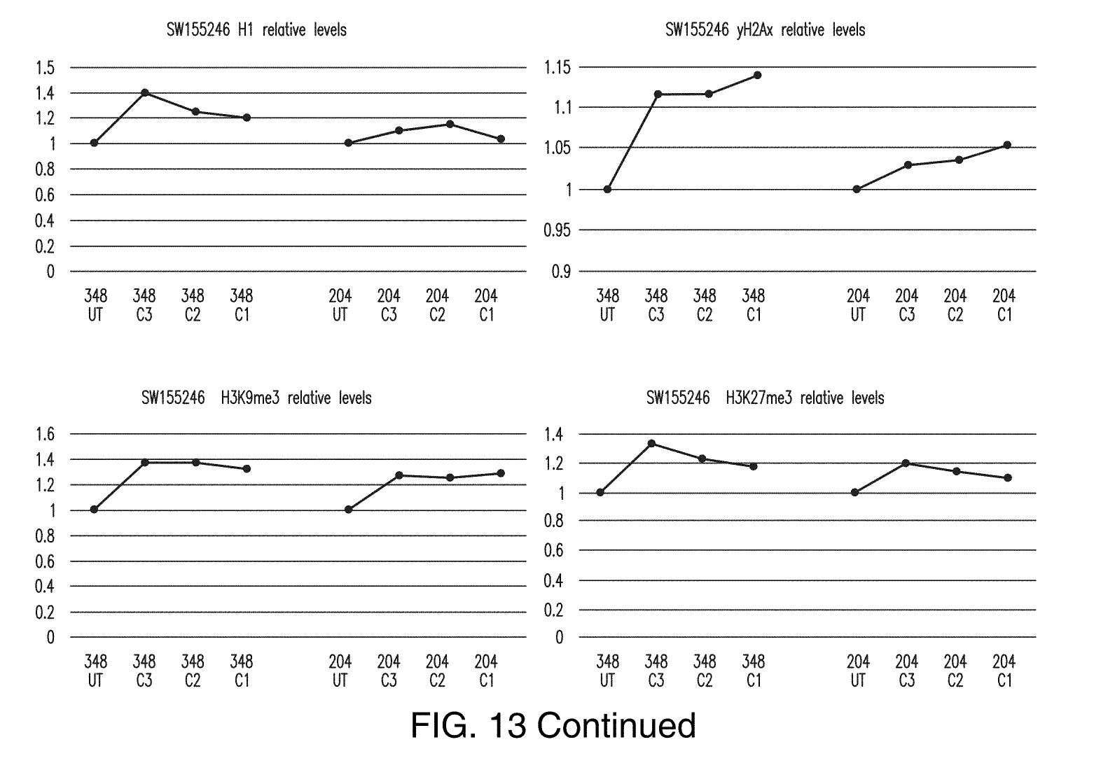

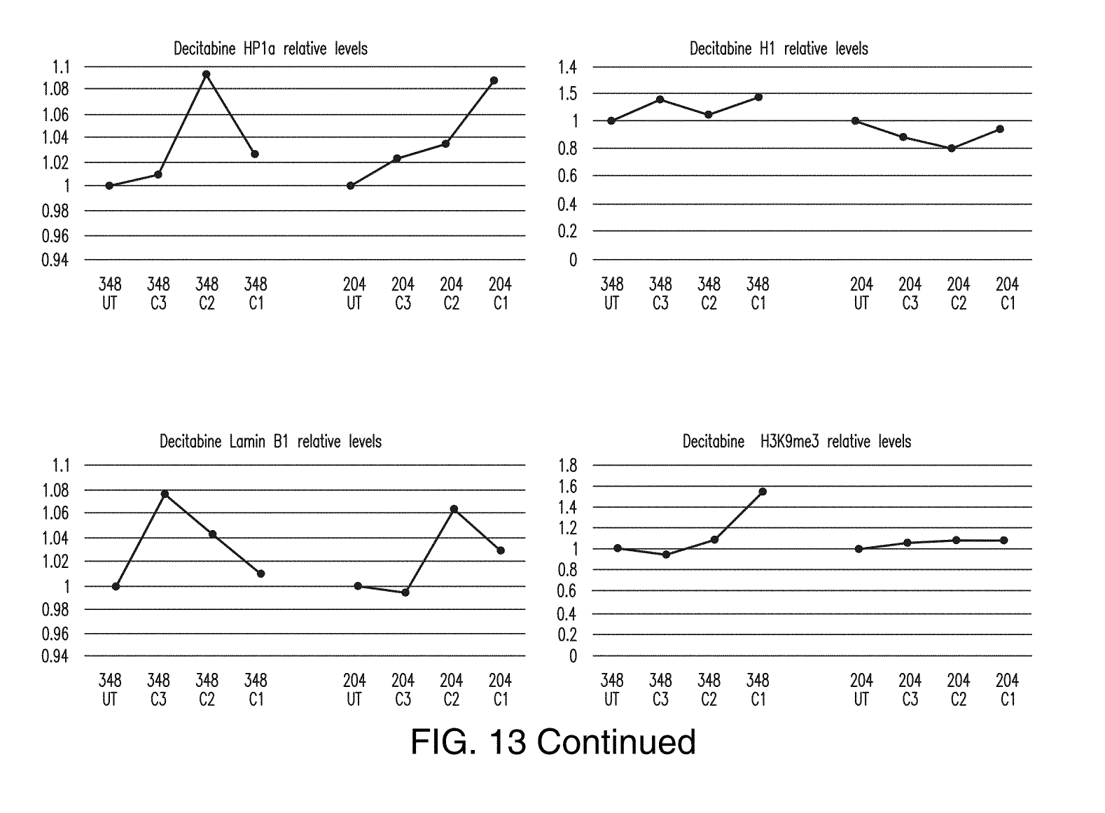

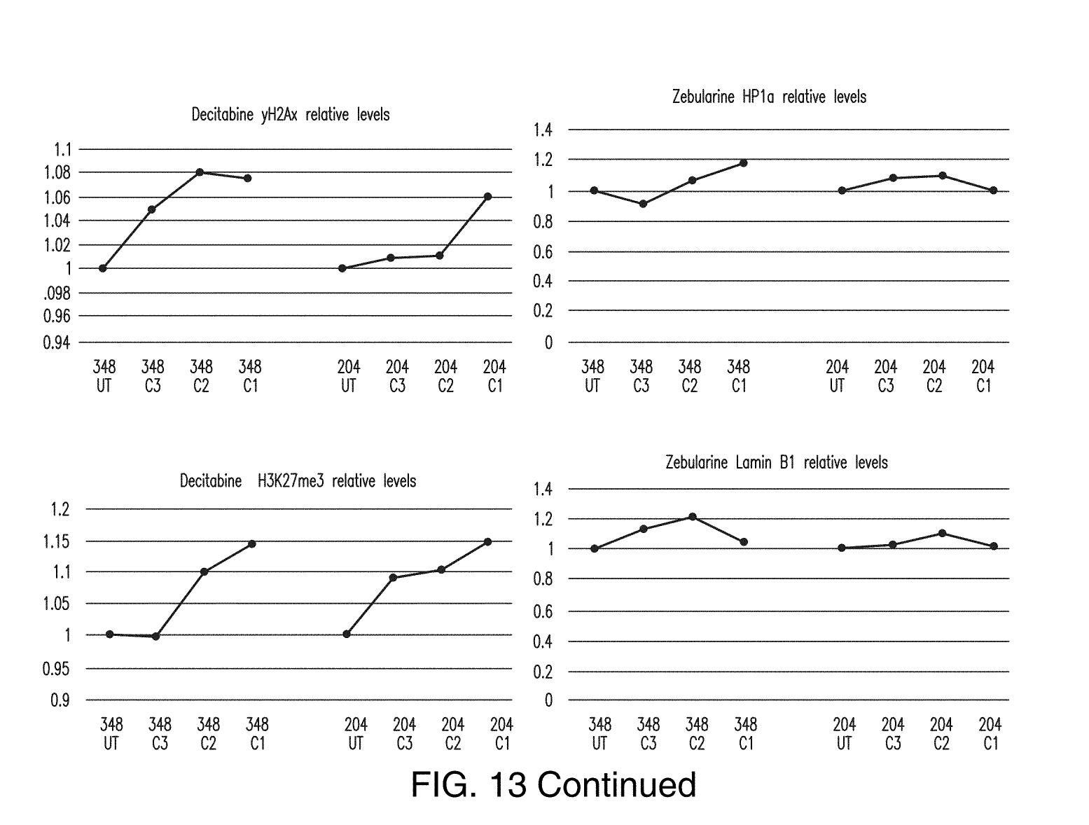

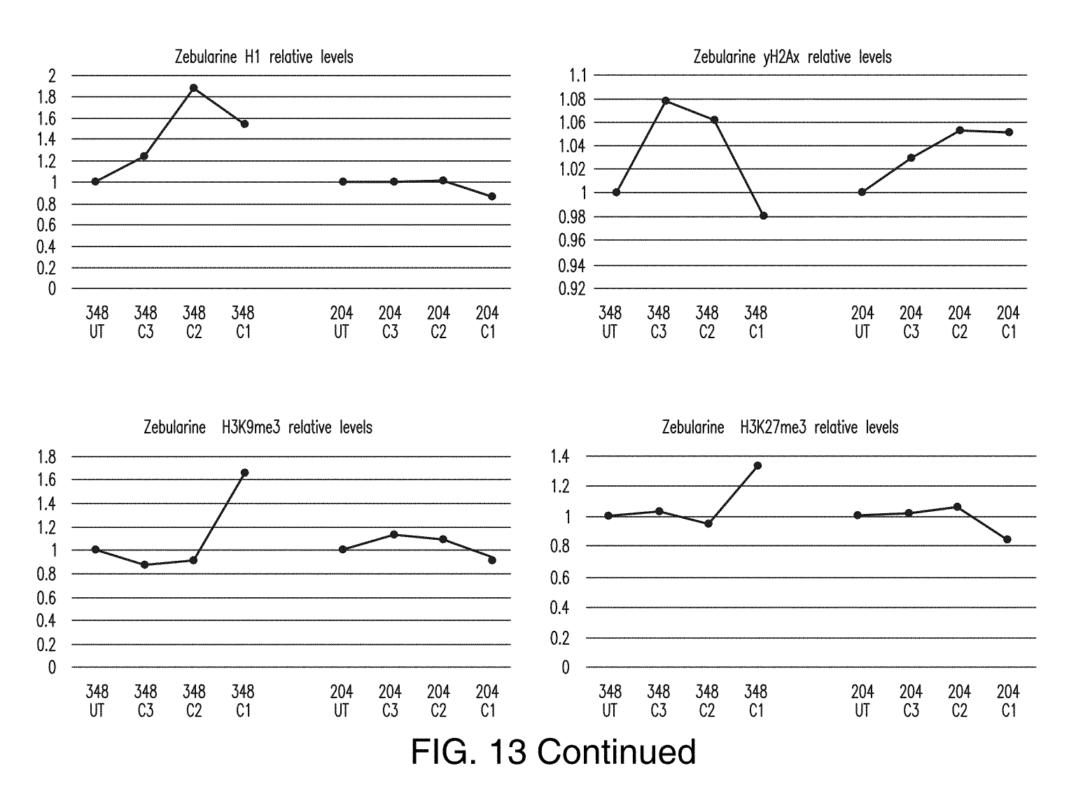

With regard to Decitabine, Zebularine, SW155246, and Chaetocin treatments, these four compounds modulated the expression of the six markers tested (FIG. 13 and FIG. 14). In cells treated with Decitabine, Zebularine and SW155246, in some cases there was an inverted U shaped curve of youthful marker expression associated with treatment with these compounds. In these cases, the highest dose of the drug resulted in a lower level of expression of some of the five markers, and the middle or lower dose resulted in a higher level of expression (FIG. 13). As noted above, an increase in expression of the markers, can occur at concentrations where the compounds are toxic to the cells.

### Long Term Treatment Results in Cell Depletion

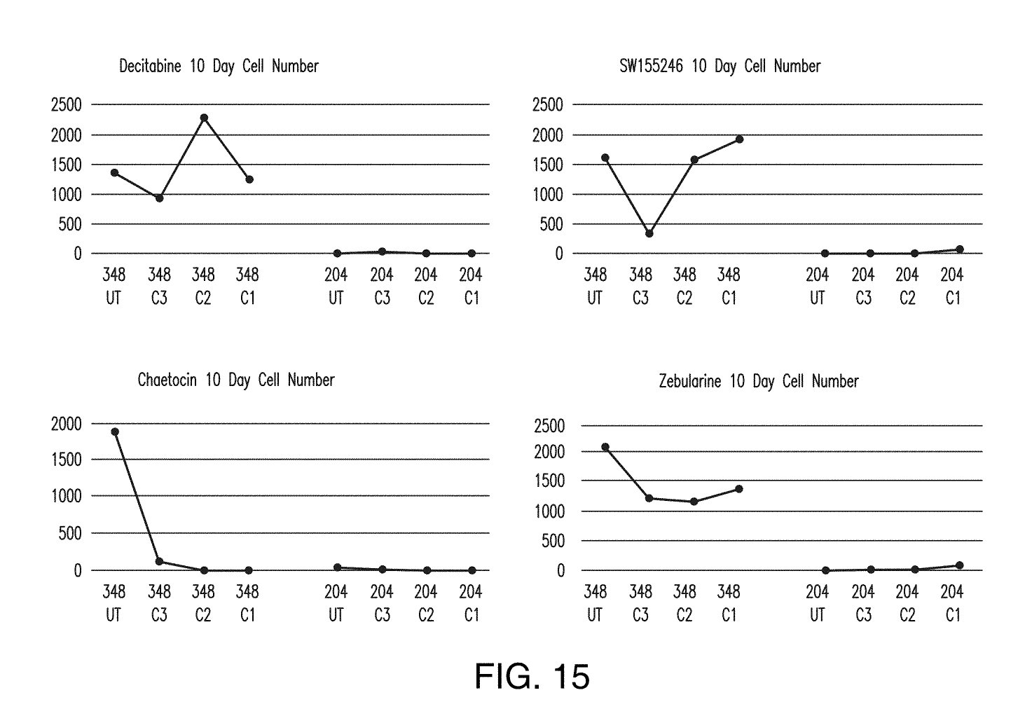

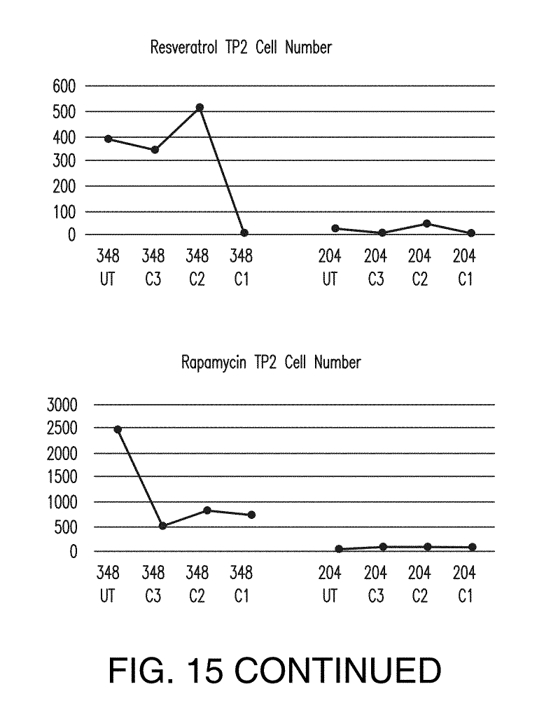

To determine if prolonged culture with the compounds resulted in toxic effects, cells were cultured for 10 days with the compounds. Control cultures of old cells had largely died at that time point, and the drugs did not rescue them (FIG. 15). Control cultures of young cells were still healthy after 10 days, and the compounds all had effects on viability (FIG. 15). The highest toxicity was seen with Chaetocin. Toxicity was also seen with Rapamycin and Resveratrol. Much less toxicity was seen with Decitabine, Zeburaline and SW155246.

Treatment with DNMT Inhibitors Modulates Global DNA Methylation Levels

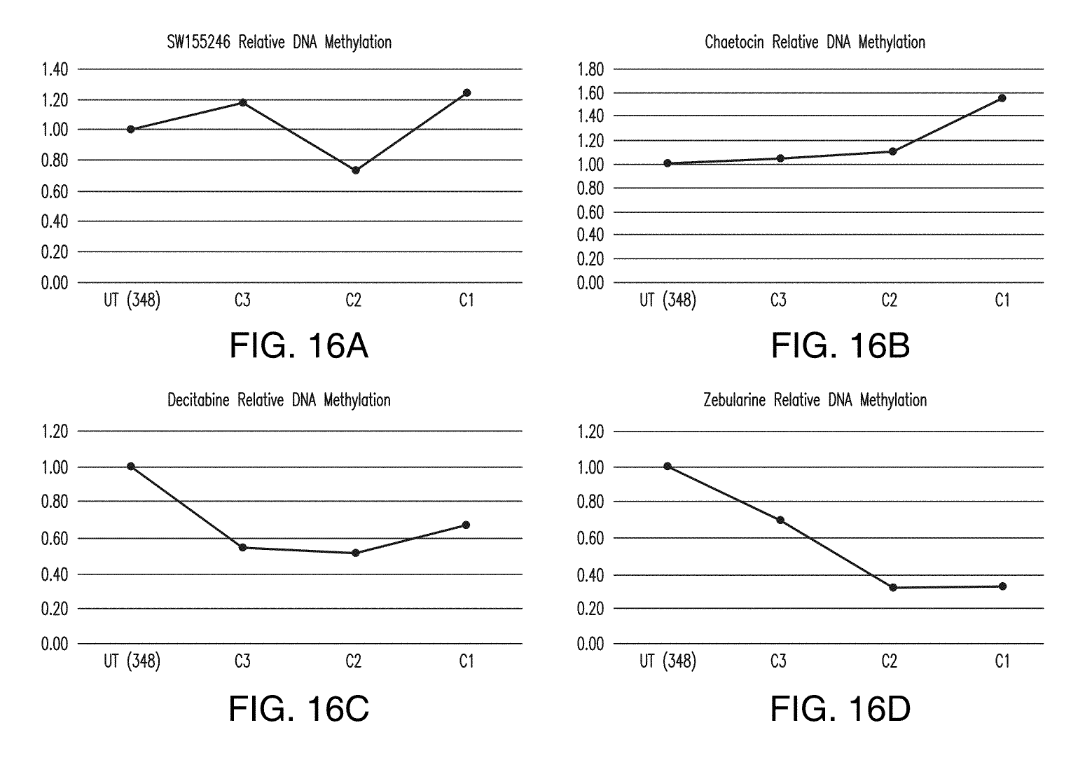

The effect of the compounds on global 5-mC DNA methylation levels were assessed after 3 days of treatment with Decitabine, Zebularine, Chaetocin and SW155246. For cells treated with SW155246, the selective DNMT1 inhibitor, levels of DNA methylation increased with both the highest and lowest concentrations, but decreased slightly with the middle concentration (FIG. 16a). Levels of DNA methylation for cells treated with Chaetocin, the SUV3/9 inhibitor, increased steadily based on concentration (FIG. 16b). For both Zebularine and Decitabine, inhibitors of both DNM1 and DNMT3a/b, global levels of DNA methylation decreased with treatment (FIG. 16c,d).

### Example 14

### iPSC-Derived Midbrain Dopamine Neurons Rely More Heavily on Mitochondria as they Mature

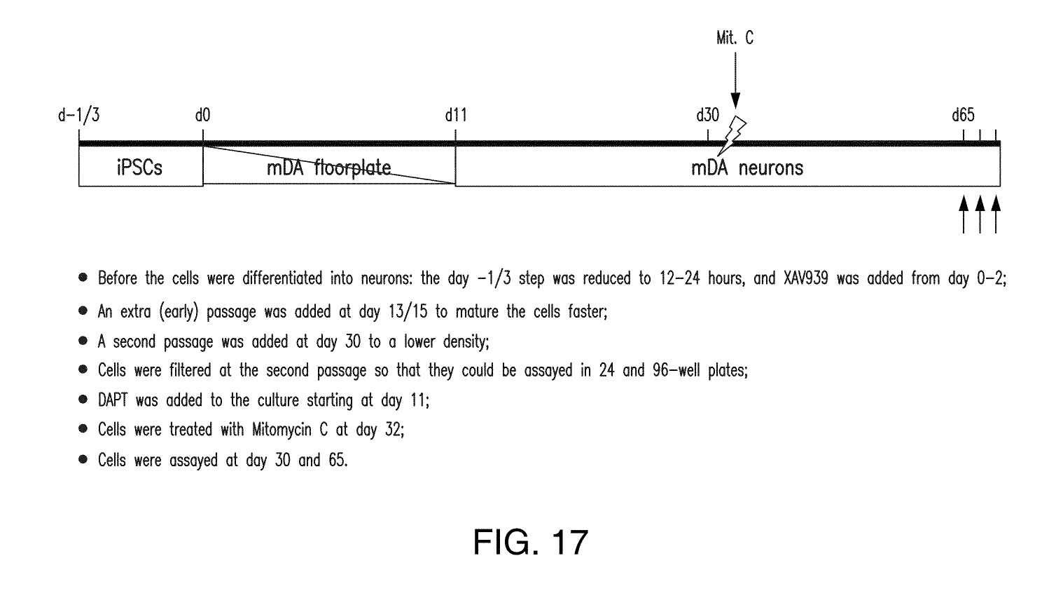

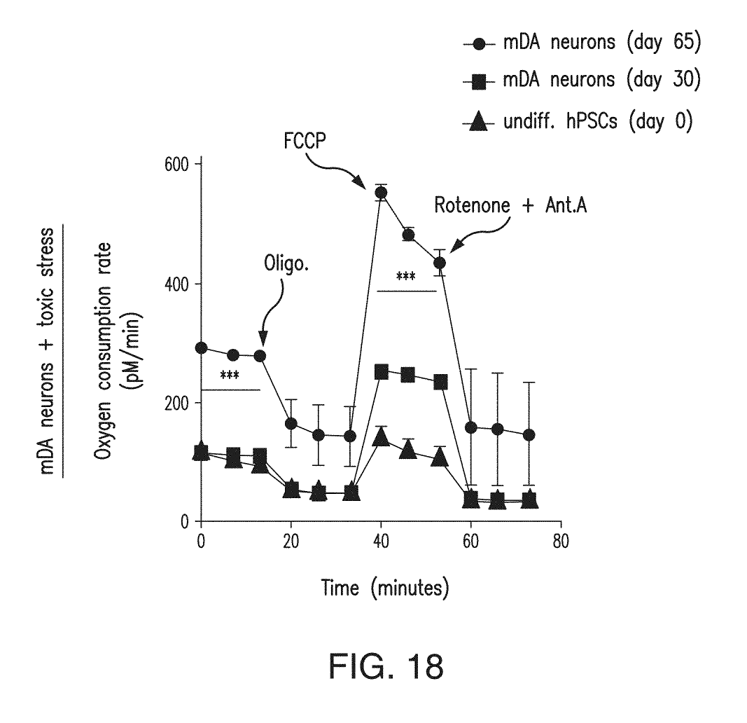

iPSCs were differentiated into midbrain dopamine neurons (mDA) as described by Kriks et al., Nature. 2011 Nov. 6; 480(7378):547-51 and Miller et al., Cell Stem Cell. 2013 Dec. 5; 13(6):691-705, wherein the methods were modified as shown in FIG. 17. Specifically, iPSCs were cultured for 12-24 hours (culture days −0 to −2) before differentiation of the cells into mDA, and the wingless (Wnt) signaling inhibitor XAV939 was added to the cell culture from days 0-2 when differentiating the iPSCs into mDA. The mDA cells were subjected to passage at days 13 and/or 15 and 30 of culture, wherein the cells were filtered and plated at a lower density in the day 30 passage. DAPT (N-[(3,5-Difluorophenyl)acetyl]-L-alanyl-2-phenyl]glycine-1,1-dimethylethyl ester) was added to the culture beginning at day 11, and the cells were treated with mitomycin C for 1 hour at day 32. Cells were then assayed for oxygen consumption in the presence of the mitochondrial stressors rotenone or carbonilcyanide p-triflouromethoxyphenylhydrazone (FCCP) at day 65 and 30. Undifferentiated iPSCs (culture day 0) were used as controls. As shown in FIG. 18, mDA cultured to 65 days exhibited greater oxygen consumption under the stressed conditions compared to the 30 day cultured mDA and undifferentiated iPSC controls.

### REFERENCES

Agrawal, A., Murphy, R. F., and Agrawal, D. K. 2007. DNA methylation in breast and colorectal cancers. Mod. Pathol. 20:711-721.
Ahuja, N., and Issa, J. P. 2000. Aging, methylation and cancer. Histol. Histopathol. 15:835-842.
Allis, C. D., Jenuwein, T., and Reinberg, D. 2007. Epigenetics. Cold Spring Harbor: Cold Spring Harbor Laboratory Press.
Arita, K., Ariyoshi, M., Tochio, H., Nakamura, Y., and Shirakawa, M. 2008. Recognition of hemimethylated DNA by the SRA protein UHRF1 by a base flipping mechanism. Nature 455: 818-821.
Aravin A A, Hannon G J, Brennecke J. The PIWI-piRNA pathway provides an adaptive defense in the transposon arms race. Science. 2007 Nov. 2; 318(5851):761-4.
Arnaud, M., Dante, R., and Niveleau, A. 1985. DNA methyltransferases in normal and avian sarcoma virus-transformed rat cells. Quantitation of 5-methyldeoxycytidine in DNA and enzyme kinetics study. Biochim. Biophys. Acta 826:108-112.
Avvakumov, G. V., Walker, J. R., Xue, S., Li, Y., Duan, S., Bronner, C., Arrowsmith, C. H., and Dhe-Paganon, S. 2008. Structural basis for recognition of hemimethylated DNA by the SRA domain of human UHRF1. Nature 455:822-825.
Bandyopadhyay, D., and Medrano, E. E. 2003. The emerging role of epigenetics in cellular and organismal aging. Exp. Gerontol. 38:1299-1307.
Barbot, W., Dupressoir, A., Lazar, V., and Heidmann, T. 2002. Epigenetic regulation of an TAP retrotransposon in the aging mouse: progressive demethylation and de-silencing of the element by its repetitive induction. Nucleic Acids Res. 30:2365-2373.
Bedford, M. T., and van Helden, P. D. 1987. Hypomethylation of DNA in pathological conditions of the human prostate. Cancer Res. 47:5274-5276.
Berdishev, G. D., Korotaev, G. K., Bojarskikh, G. V., and Vanyushin, B. F. 1967. Nucleotide composition of DNA and RNA from somatic tissues of humpback salmon and its changes during spawning. Biochemistry (Mosc.) 32:988-993.
Bertram, C., and Hass, R. 2008. Cellular responses to reactive oxygen species-induced DNA damage and aging. Biol. Chem. 389:211-220.
Bestor, T. H. 2000. The DNA methyltransferases of mammals. Hum. Mol. Genet. 9:2395-2402.
Casillas, M. A., Lopatina, N., Andrews, L. G., and Tollefsbol, T. O. 2003. Transcriptional control of the DNA methyltransferases is altered in aging and neoplastically-transformed human fibroblasts. Mol. Cell. Biochem. 252:33-43.
Castro, R., Rivera, I., Struys, E. A., Jansen, E. E. W., Ravasco, P., Camilo, M. E., Blom, H. J., Jakobs, C., and de Almeida, I. T. 2003. Increased homocysteine and S-adenosylhomocysteine concentrations and DNA hypomethylation in vascular disease. Clin. Chem. 49:1292-1296.
Cerda, S., and Weitzman, S. A. 1997. Influence of oxygen radical injury on DNA methylation. Mutat. Res. 386:141-152.
Chen, J. H., Hales, C. N., and Ozanne, S. E. 2007. DNA damage, cellular senescence and organismal ageing: causal or correlative? Nucleic Acids Res. 35:7417-7428.
Chen, R. Z., Pettersson, U., Beard, C., Jackson-Grusby, L., and Jaenisch, R. 1998. DNA hypomethylation leads to elevated mutation rates. Nature 395:89-93.
Chen, Z., Karaplis, A. C., Ackerman, S. L., Pogribny, I. P., Melnyk, S., Lussier-Cacan, S., Chen, M. F., Pai, A., John, S. W., Smith, R. S., Bottiglieri, T., Bagley, P., Selhub, J., Rudnicki, M. A., James, S. J., and Rozen, R. A. 2001. Mice deficient in methylenetetrahydrofolate reductase exhibit hyperhomocysteinemia and decreased methylation capacity, with neuropathology and aortic lipid deposition. Hum. Mol. Genet. 10:433-443.
Chiang, P. K., Gordon, R. K., Tal, J., Zeng, G. C., Doctor, B. P., Pardhasaradhi, K., and McCann, P. P. 1996. S-Adenosylmethionine and methylation. FASEB J. 10:471-480.
Coleman, W. B., and Tsongalis, G. J. 2006. Molecular mechanisms of human carcinogenesis. EXS. 96:321-349.
Creusot, F., Acs, G., and Christman, J. K. 1982. Inhibition of DNA methyltransferase and induction of Friend erythroleukemia cell differentiation by 5-azacytidine and 5-aza-2-deoxycytidine. J. Biol. Chem. 257:2041-2048.
Das, H. K. 2008. Transcriptional regulation of the presenilin-1 gene: implication in Alzheimer's disease. Front. Biosci. 13:822-832.
Dennis, K., Fan, T., Geiman T., Yan, Q., and Muegge, K. 2001. Lsh, a member of the SNF2 family, is required for genome-wide methylation. Genes Dev. 15:2940-2944.
DePinho, R. A. 2000. The age of cancer. Nature 408:248-254.
Dong, C., Yoon, W., and Goldschmidt-Clermont, J. 2002. DNA methylation and atherosclerosis. J. Nutr. 132:2406S-2409S.
Drinkwater, R. D., Blake, T. J., Morley, A. A., and Turner, D. R. 1989. Human lymphocytes aged in vivo have reduced levels of methylation in transcriptionally active and inactive DNA. Mutat. Res. 219:29-37.
Dunn, B. K. 2003. Hypomethylation: one side of a larger picture. Ann. NY. Acad. Sci. 983:28-42.
Eden, A., Gaudet, F., Waghmare, A., and Jaenisch, R. 2003. Chromosomal instability and tumors promoted by DNA hypomethylation. Science 300:455.
Egger, G., Liang, G., Aparicio, A., and Jones, P. A. 2004. Epigenetics in human disease and prospects for epigenetic therapy. Nature 429:457-463.
Egger Gl, Jeong S, Escobar S G, Cortez C C, Li T W, Saito Y, Yoo C B, Jones P A, Liang G. 2006. Identification of DNMT1 (DNA methyltransferase 1) hypomorphs in somatic knockouts suggests an essential role for DNMT1 in cell survival. Proc Natl Acad Sci USA. 2006 Sep. 19; 103(38):14080-5. Epub 2006 Sep. 8.
Ehrlich, M. 2006. Cancer-linked DNA hypomethylation and its relationship to hypermethylation. Curr. Top. Microbiol. Immunol. 310:251-274.
Ehrlich, M., Woods, C. B., Yu, M. C., Dubeau, L., Yang, F., Campan, M., Weisenberger, D. J., Long, T., Youn, B., Fiala, E. S., and Laird, P. W. 2006. Quantitative analysis of associations between DNA hypermethylation, hypomethylation, and DNMT RNA levels in ovarian tumors. Oncogene 25:2636-2645.
Fairweather, D. S., Fox, M., and Margison, G. P. 1987. The in vitro lifespan of MRC-5 cells is shortened by 5-azacytidine-induced demethylation. Exp. Cell Res. 168:153-159.
Fallest-Strobl, P. C., Koch, D. D., Stein, J. H., and McBribe, P. E. 1997. Homocysteine: a new risk factor for atherosclerosis. Am. Fam. Physician. 56:1607-1612.
Feinberg, A. P. 2007. Phenotypic plasticity and the epigenetics of human disease. Nature 447: 433-440.
Feinberg, A. P., and Vogelstein, B. 1983. Hypomethylation distinguishes genes of some human cancers from their normal counterparts. Nature 301:89-92.
Feinberg, A. P., Gehrke, C. W., Kuo, K. C., and Ehrlich, M. 1988. Reduced genomic 5-methylcytosine in human colonic neoplasia. Cancer Res. 48:1159-1161.
Feinberg, A. P., and Tycko, B. 2004. The history of cancer epigenetics. Nat. Rev. Cancer 4:143-153.
Feinberg, A. P. 2008. Epigenetics at the epicenter of modern medicine. JAMA 2999:1345-1350.
Flatau, E., Bogenmann, E., and Jones, P. A. 1983. Variable 5-methylcytosine levels in human tumor cell lines and fresh pediatric tumor explants. Cancer Res. 43:4901-4905.
Fraga, M. F., Herranz, M., Espada, J., Ballestar, E., Paz, M. F., Ropero, S., Erkek, E., Bozdogan, O., Peinado, H., Niveleau, A., Mao, J. H., Balmain, A., Cano, A., and Esteller, M. 2004. A mouse skin multistage carcinogenesis model reflects the aberrant DNA methylation patterns of human cancers. Cancer Res. 64:5527-5534.
Fraga, M. A., and Esteller, M. 2007. Epigenetics and aging: the targets and the marks. Trends Genet. 23:413-418.
Fuso, A., Seminara, L., Cavallaro, R. A., D'Anselmi, F., and Scarpa, S. 2005. S-adenosylmethionine/homocysteine cycle a with consequent deregulation of PS1 and BACE and beta-amyloid production. Mol. Cell. Neurosci. 28:195-204.
Fuso, A., Cavallaro, R. A., Zampelli, A., D'Anselmi, F., Piscopo, P., Confaloni, A., and Scarpa, S. 2007. gamma-Secretase is differentially modulated by alterations of homocysteine cycle in neuroblastoma and glioblastoma cells. J. Alzheimer's Dis. 11:275-290.
Gama-Sosa, M. A., Slagel, V. A., Trewyn, R. W., Oxenhandler, R., Kuo, K. C., Gehrke, C. M., and Erhlich, M. 1983a. The 5-methylcytosine content of DNA from human tumors. Nucleic Acids Res. 11:6883-6894.
Gama-Sosa, M. A., Wang, R. Y., Kuo, K. C., Gehrke, C. W., and Ehrlich, M. 1983b. The 5-methylcytosine content of highly repeated sequences in human DNA. Nucleic Acids Res. 11:3087-3095.
Gaudet, F., Hodgson, J. G., Eden, A., Jackson-Grusby, L., Dausman, J., Gray, J. W., Leonhardt, H., and Jaenisch, R. 2003. Induction of tumors in mice by genomic hypomethylation. Science 300:489-492.
Goll, M. G., and Bestor, T. H. 2005. Eukaryotic cytosine methyltransferases. Annu. Rev. Biochem. 74:481-514.
Gorbunova, V., Seluanov, A., Mao, Z., and Hine, C. 2007. Changes in DNA repair during aging. Nucleic Acids Res. 35:7466-7474.
Goronzy, J. J., and Weyand, C. M. 2003. Aging, autoimmunity and arthritis: T-cell senescence and contraction T-cell repertoire diversity—catalysts of autoimmunity and chronic inflammation. Arthritis Res. Ther. 5:225-234. Age-Related Genomic Hypomethylation 23
Gowher, H., Liebert, K., Hermann, A., Xu, G., and Jeltsch, A. 2005. Mechanism of stimulation of catalytic activity of Dnmt3A and Dnmt3B DNA-(cytosine-05)-methyltransferases by Dnmt3L. J. Biol. Chem. 280:13341-13348.
Grunau, C., Brun, M. E., Rivals, I., Hindermann, W., Favre-Mercuret, M., Granier, G., and DeSario, A. 2008. BAGE hypomethylation, a new epigenetic biomarker for colon cancer detection. Cancer Epidemiol. Biomarkers Prev. 17:1374-1379.
Guerrero-Preston, R., Santella, R. M., Blanco, A., Desai, M., Berdasco, M., and Fraga, M. Global DNA hypomethylation in liver cancer cases and controls: a phase I preclinical biomarker development study. Epigenetics 2:223-226.
Hamm, S., Just, G., Lacoste, N., Moitessier, N., Szyf, M., and Mamer, O. 2008. On the mechanism of demethylation of 5-methylcytosine in DNA. Bioorg. Med. Chem. Lett. 18:1046-1049.
Hardy, J., and Selkoe, D. J. 2002. The amyloid hypothesis of Alzheimer's disease: progress and problems on the road to therapeutics. Science 297:353-356.
Hashimoto, H., Horton, J. R., Zhang, X., Bostick, M., Jacobsen, S. E., and Cheng, X. 2008. The SRA domain of UHRF1 flips 5-methylcytosine out of the DNA helix. Nature 455:826-829.
Heyn H, Li N, Ferreira H J, Moran S, Pisano D G, Gomez A, Diez J, Sanchez-Mut J V, Setien F, Carmona F J, Puca A A, Sayols S, Pujana M A, Serra-Musach J, Iglesias-Platas I, Formiga F, Fernandez A F, Fraga M F, Heath S C, Valencia A, Gut I G, Wang J, Esteller M. Distinct DNA methylomes of newborns and centenarians. Proc Natl Acad Sci USA. 2012 Jun. 26; 109(26):10522-7.
Hiltunen, M. O., Turunen, M. P., Häkkinen, T. P., Rutanen, J., Hedman, M., Mäkinen, K., Turunen, A. M., Aalto-Setälä, K., and Ylä-Herttuala, S. 2002. DNA hypomethylation and methyltransferase expression in atherosclerotic lesions. Vasc. Med. 7:5-11.
Hiltunen, M. O., and Ylä-Herttuala, S. 2003. DNA methylation, smooth muscle cells, and atherogenesis. Arterioscler. Thromb. Vasc. Biol. 23:1750-1753.
Hoffmann, M. J., and Schulz, W. A. 2005. Causes and consequences of DNA hypomethylation in human cancer. Biochem. Cell. Biol. 83:296-321.
Holliday, R. 1985. The significance of DNA methylation in cellular aging. Basic Life Sci.
35: 269-283.
Holliday, R. 1986. Strong effects of 5-azacytidine on the in vitro lifespan of human diploid fibroblasts. Exp. Cell Res. 166:543-552.
Holliday, R. 1987. The inheritance of epigenetic defects. Science 238:163-170.
Hornsby, P. J., Yang, L., and Gunter, L. E. 1992. Demethylation of satellite I DNA during senescence of bovine adrenocortical cells in culture. Mutat. Res. 275:13-19.
Howard, G., Eiges, R., Gaudet, F., Jaenisch, R., and Eden, A. 2008. Activation and transposition of endogenous retroviral elements in hypomethylation induced tumors in mice. Oncogene 27: 404-408.
Horvath, S. DNA methylation age of human tissues and cell types. Genome Biol 14, R115 (2013).
Issa, J. P. 2003. Age-related epigenetic changes and the immune system. Clin. Immunol. 109: 103-108.
Jager, R. D., Mieler W. F., and Miller, J. W. 2008. Age-related macular degeneration. N. Engl. J. Med. 358:2606-2617.
James, S. J., Melnyk, S., Pogribna, M., Pogribny, I. P., and Caudill, M. A. 2002. Elevation of S-adenosylhomocysteine and DNA hypomethylation: potential epigenetic mechanism for homocysteine-related pathology. J. Nutr. 132:2361S-2366S.
Jones, P. A., and Baylin, S. B. 2007. The epigenomics of cancer. Cell 128:683-692.
Juliano C, Wang J, Lin H. Uniting germline and stem cells: the function of PIWI proteins and the piRNA pathway in diverse organisms. Annu Rev Genet. 2011; 45:447-69.
Kangaspeska, S., Stride, B., Métivier, R., Polycarpou-Schwarz, M., Ibberson, D., Carmouche, R. P., Benes, V., Gannon, F., and Reid, G. 2008. Transient cyclical methylation of promoter DNA. Nature 452:112-115.
Kaplan, M. J., Lu, Q., Wu, A., Attwood, J., and Richardson, B. 2004. Demethylation of promoter regulatory elements contributes to perforin overexpression in CD4+ lupus T cells. J. Immunol. 172:3652-3661.
Karpf, A. R., and Matsui, S. 2005. Genetic disruption of cytosine DNA methyltransferase enzymes induces chromosomal instability in human cancer cells. Cancer Res. 65:8635-8639.
Kimura, H., and Shiota, K. 2003. Methyl-CpG-binding protein, MeCP2, is target molecule for maintenance DNA methyltransferase, Dnmtl. J. Biol. Chem. 278:4806-4812.
Kuo, H. K., Sorond, F. A., Chen, J. H., Hashmi, A., Milberg, W. P., and Lipsitz, L. A. 2005. The role of homocysteine in multisystem age-related problems: a systematic review. J. Gerontol. A Biol. Sci. Med. Sci. 60:1190-1201.24
Kuramochi-Miyagawa S, Watanabe T, Gotoh K, Totoki Y, Toyoda A, Ikawa M, Asada N, Kojima K, Yamaguchi Y, Ijiri T W, Hata K, Li E, Matsuda Y, Kimura T, Okabe M, Sakaki Y, Sasaki H, Nakano T. DNA methylation of retrotransposon genes is regulated by PIWI family members MILI and MIWI2 in murine fetal testes. Genes Dev. 2008 Apr. 1; 22(7):908-17.
I. P. Pogribny and B. F. Vanyushin Laner, T., Schultz, W. A., Engers, R., Müller, M., and Florl, A. R. 2005. Hypomethylation of the XIST gene promoter in prostate cancer. Oncol. Res. 15:257-264.
Li, E., Bestor, T. H., and Jaenisch, R. 1992. Targeted mutation of the DNA methyltransferase gene results in embryonic lethality. Cell 69:915-926.
Li, G., Weyand, C., and Goronzy, J. J. 2008. Epigenetic mechanisms of age-dependent KIR2DL4 expression in T cells. J. Leukoc. Biol. 84:824-834.
Liang, G., Chan, M. F., Tomigahara, Y., Tsai, Y. C., Gonzales, F. A., Li, E., Laird, P. W., and Jones, P. A. 2002. Cooperativity between DNA methyltransferases in the maintenance methylation of repetitive elements. Mol. Cell. Biol. 22:480-491.
Liu, C., Xu, D., Sjöberg, J., Forsell, P., Björkholm, M., and Claesson, H. E. 2004. Transcriptional regulation of 15-lipoxygenase expression by promoter methylation. Exp. Cell Res. 297:61-67.
Lopatina, N., Haskell, J. F., Andrews, L. G., Poole, J. C., Saldanha, S., and Tollefsbol, T. 2002. Differential maintenance and de novo methylating activity by three DNA methyltransferases in aging and immortalized fibroblasts. J. Cell. Biochem. 84:324-334.
Loriot, A., De Plaen, E., Boon, T., and De Smet, C. 2006. Transient down-regulation of DNMT1 methyltransferase leads to activation and stable hypomethylation of MAGE-A1 in melanoma cells. J. Biol. Chem. 281:10118-10126.
Lu, Q., Kaplan, M., Ray, D., Ray, D., Zacharek, S., Gutsch, D., and Richardson, B. 2002. Demethylation of INGAL (CD11a) regulatory sequences in systemic lupus erythematosus. Arthritis Rheum. 46:1282-1291.
Lund, G., Andersson, L., Lauria, M., Lindholm, M., Fraga, M. F., Villar-Garea, A., Ballestar, E., Esteller, M., and Zaina, S. 2004. DNA methylation polymorphisms precede any histological sign of atherosclerosis in mice lacking apolipoprotein E. J. Biol. Chem. 279: 29147-29154.
Mason, J. B. 2003. Biomarkers of nutrient exposure and status in one-carbon (methyl) metabolism. J. Nutr. 133:941S-947S.
Mays-Hoopes, L. L., Brown, A., and Huang, R. C. C. 1983. Methylation and rearrangement of mouse intracisternal A particle genes in development, adding, and myeloma. Mol. Cell. Biol. 3: 1371-1380.
Mensah, G. A., and Brown, D. W. 2007. An overview of cardiovascular disease burden in the United States. Health Aff. (Millwood). 26:38-48.
Métivier, R., Gallais, R., Tiffoche, C., Le Péron, C., Jurkowska, R. Z., Carmouche, R. P., Ibberson, D., Barath, P., Demay, F., Reid, G., Benes, V., Jeltsch, A., Gannon, F., and Salbert, G. 2008. Cyclical DNA methylation of a transcriptionally active promoter. Nature 452:45-50.
Miller J D, Ganat Y M, Kishinevsky S, Bowman R L, Liu B, Tu E Y, Mandal P K, Vera E,
Shim J W, Kriks S, Taldone T, Fusaki N, Tomishima M J, Krainc D, Milner T A, Rossi D J, Studer L. Cell Stem Cell. 2013 Dec. 5; 13(6):691-705. doi: 10.1016/j.stem.2013.11.006.
Moore, L. E., Pfeiffer, R. M., Poscablo, C., Real, F. X., Kogevinas, M., Silverman, D., Garcia-Closas, R., Chanock, S., Tardón, A., Serra, C., Carrato, A., Dosemeci, M., Garcia-Closas, M., Esteller, M., Fraga, M., Rothman, N., and Malats, N. 2008. Genomic DNA hypomethylation as a biomarker for bladder cancer susceptibility in the Spanish Bladder Cancer Study: a casecontrol study. Lancet Oncol. 9:359-366.
Morris, M. S. 2003. Homocysteine and Alzheimer's disease. Lancet Neurol. 2:425-428
Myant, K., and Stancheva, I. 2008. LSH cooperates with DNA methyltransferases to repress transcription. Mol. Cell. Biol. 28:215-226.
Nehler, M. R., Taylor, L. M., Jr., and Porter, J. M. 1997. Homocysteinemia as a risk factor for atherosclerosis: a review. Cardiovasc. Surg. 5:559-567.
Newman, P. E. 1999. Can reduced folic acid and vitamin B12 levels cause deficient DNA methylation producing mutations which initiate atherosclerosis? Med. Hypothesis 53:421-424.
Nguyen, S., Meletis, K., Fu, D., Jhaveri, S., and Jaenisch, R. 2007. Ablation of de novo DNA methyltransferase Dnmt3a in the nervous system leads to neuromuscular defects and shortened lifespan. Dev. Dyn. 236:1663-1676.
Niculescu, M. D., and Zeisel, S. H. 2002. Diet, methyl donors and DNA methylation: interactions between dietary folate, methionine and choline. J. Nutr. 132:2333S-2335S.
Nijhout, H. F., Reed, M. C., Anderson, D. F., Mattingly, J. C., James, S. J., and Ulrich, C. M. 2006. Long-range allosteric interactions between the folate and methionine cycles stabilize DNA methylation reaction rate. Epigenetics, 1:81-87.
Okano, M., Bell, D. W., Haber, D. A., and Li, E. 1999. DNA methyltransferases Dnmt3a and Dnmt3b are essential for de novo methylation and mammalian development. Cell 99: 247-257.
Ono, T., Shinya, K., Uehara, Y., and Okada, S. 1989. Endogenous virus genomes become hypomethylated tissue-specifically during aging process of C57BL mice. Mech. Ageing Dev. 50:27-36.
Ono, T., Takahashi, N., and Okada, S. 1989a. Age-associated changes in DNA methylation and mRNA level of the c-myc gene in spleen and liver of mice. Mutat. Res. 219:39-50.
Ono, T., Uehara, Y., Kurishita, A., Tawa, R., and Sakurai, H. 1993. Biological significance of DNA methylation in the ageing process. Age Ageing. 22:S34-S43.
Oomen, A. M., Griffin, J. B., Sarath, G., and Zempleni, J. 2005. Roles of nutrients in epigenetic events. J. Nutr. Biochem. 16:74-77.
Pastorino, L., and Lu, K. P. 2006. Pathogenic mechanisms in Alzheimer's disease. Eur. J. Pharmacol. 545:29-38.
Peng J C, Lin H. Beyond transposons: the epigenetic and somatic functions of the PIWI-piRNA mechanism. Curr Opin Cell Biol. 2013 April; 25(2):190-4.
Pogribny, I. P., Tryndyak, V. P., Boureiko, A., Melnyk, S., Bagnyukova, T. V., Montgomery, B., and Rusyn, I. 2008. Mechanisms of peroxisome proliferator-induced DNA hypomethylation in rat liver. Mut. Res. 644:17-23.
Prelog, M., 2006. Aging of the immune system: a risk factor for autoimmunity? Autoimmun. Rev. 5:136-139.
Raines, E. W., and Ross, R. 1995. Biology of atherosclerotic plaque formation: possible role of growth factor lesion development and the potential impact of soy. J. Nutr. 125: 624S-630S.
Ravindran, C. R., and Ticku, M. K. 2005. Methylation of NMDA receptor NR2B gene as a function of age in the mouse brain. Neurosci. Lett. 380:223-228.
Ray, D., Wu, A., Wilkinson, J. E., Murphy, H. S., Lu, Q., Kluve-Beckerman, B., Liepnieks, J. J., Benson, M., Yung, R., and Richardson, B. 2006. Aging in heterozygous Dnmtl-deficient mice: effects on survival, the DNA methylation genes, and the development of amyloidosis. J. Gerontol. A Biol. Sci. Med. Sci. 61:115-124.
Refsum, H., Nurk, E., Smith, A. D., Ueland, P. M., Gjesdal C. G., Bjelland, I., Tverdal, A., Tell, G. S., Nygård, O., and Vollset, S. E. 2006. The Hordaland Homocysteine Study: a communitybased study of homocysteine, its determinants, and associations with disease. J. Nutr. 136: 1731S-1740S.
Richardson, B. C. 2002. Role of DNA methylation in the regulation of cell function: autoimmunity, aging and cancer. J. Nutr. 132:2401S-2405S.
Richardson, B. 2003a. DNA methylation and autoimmune disease. Clin. Immunol. 109:72-79.
Richardson, B. 2003b. Impact of aging on DNA methylation. Ageing Res. Rev. 2:245-261.
Rodriguez, J., Frigola, J., Vendrell, E., Riques, R. A., Fraga, M. F., Morales, C., Moreno, V., Esteller, M., Capellá, G., Ribas, M., and Peinado, M. A. 2006. Chromosomal instability correlates with genome-wide DNA demethylation in human primary colorectal cancers. Cancer Res. 66:8462-8468.
Rollins, R. A., Haghighi, F., Edwards, J. R., Das, R., Zhang, M. Q., Ju, J., and Bestor, T. H. 2006. Large-scale structure of genomic methylation patterns. Genome Res. 16:157-163.
Romanov, G. A., and Vanyushin, B. F. 1981. Methylation of reiterated sequences in mammalian DNAs. Effects of the tissue type, age, malignancy and hormonal induction. Biochim. Biophys. Acta 653: 204-218.
Ross, R. 1999. Atherosclerosis—an inflammatory disease. N. Engl. J. Med. 340:115-126.
Ross R J, Weiner M M, Lin H. PIWI proteins and PIWI-interacting RNAs in the soma. Nature. 2014 Jan. 16; 505(7483):353-9.
Rozen, R. 2000. Genetic modulation of homocysteinemia. Semin. Thromb. Hemost. 26:255-261.
Sato, N., Maitra, A., Fukushima, N., van Heek, N. T., Matsubayashi, H., Iacobuzio-Donahue, C. A., Rosty, C., and Coggins, M. 2003. Frequent hypomethylation of multiple genes overexpressed in pancreatic ductal adenocarcinoma. Cancer Res. 63:4158-4166.
Schulz, R. J. 2007. Homocysteine as a biomarker for cognitive dysfunction in the elderly. Curr. Opin. Clin. Nutr. Metab. Care. 10:718-723.
Selhub, J. 2002. Folate, vitamin B12 and vitamin B6 and one carbon metabolism. J. Nutr. Health Aging. 6:39-42. 26 I. P. Pogribny and B. F. Vanyushin
Seivwright, C., Macnab, J. C., and Adams, R. L. 1993. S-adenosylmethionine metabolism in herpes simplex virus type 2-infected cells. J. Gen. Virol. 74:1405-1407.
Simon M, Sarkies P, Ikegami K, Doebley A L, Goldstein L D, Mitchell J, Sakaguchi A, Miska E A, Ahmed S. Reduced insulin/IGF-1 signaling restores germ cell immortality to Caenorhabditis elegans PIWI mutants. Cell Rep. 2014 May 8; 7(3):762-73.
Singhal, R. P., Mays-Hoopes, L. L., and Eichhorn, G. L. 1987. DNA methylation in aging of mice. Mech. Ageing Dev. 41:199-210.
Spry, M., Scott, T., Pierce, H., and D'Orazio, J. A. 2007. DNA repair pathways and hereditary cancer susceptibility syndromes. Front. Biosci. 12:4191-4207.
Sun, L. Q., Lee, D. W., Zhang, Q., Xiao, W., Raabe, E. H., Meeker, A., Miao, D., Huso, D. L., and Arceci, R. J. 2004. Growth retardation and premature aging phenotypes in mice with disruptionof the SNF-2-like gene, PASG. Genes Dev. 18:1035-1046.
Sun, L. Q., and Arceci R. J. 2005. Altered epigenetic patterning leading to replicative senescence and reduced longevity. Cell Cycle 4:3-5.
Suuronen, T., Nuutinen, T., Ryhänen, T., Kaarniranta, K., and Salminen, A. 2007. Epigenetic regulation of clusterin/apolipoprotein J expression in retinal pigment epithelial cells. Biochem. Biophys. Res. Commun. 357:397-401.
Tohgi, H., Utsugisawa, K., Nagane, Y., Yoshimura, M., Genda, Y., and Ukitsu, M. 1999. Reduction with age in methylcytosine in the promoter region −224 approximately −101 of the amyloid precursor protein gene in autopsy human cortex. Brain Res. Mol. Brain Res. 70:288-292. Tost, J. 2008. Epigenetics. Norfolk: Caister Academic Press.
Uhl, J., Klan, N., Rose, M., Entian, K. D., Werz, O., and Steinhilber, D. 2002. The 5-lipoxygenase promoter is regulated by DNA methylation. J. Biol. Chem. 277:4374-4379.
Ulrey, C. L., Liu, L., Andrews, L. G., and Tollefsbol, T. O. 2005. The impact of metabolism on DNA methylation. Hum. Mol. Genet. 14:R139-R147.
Valinluck, V., and Sowers, L. C. 2007. Endogenous cytosine damage products alter the site selectivity of human DNA maintenance methyltransferase DNMT1. Cancer Res. 67:946-950.
Vanyushin, B. F., Korotaev, G. K., Mazin, A. L., and Berdishev, G. D. 1969. Investigation of some characteristics of the primary and secondary structure of DNA from the liver of spawning humpback salmon. Biochemistry (Mosc.) 34:191-198.
Vanyushin, B. F., Tkacheva, S. G., and Belozersky, A. N. 1970. Rare bases in animal DNA. Nature 225:948-949.
Vanyushin, B. F., Nemirovsky, L. E., Klimenko, V. V., Vasiliev, V. K., and Belozersky, A. N. 1973. The 5-methylcytosine in DNA of rats. Tissue and age specificity and the changes induced by hydrocortisone and other agents. Gerontologia 19:138-152.
Verdile, G., Gandy, S. E., and Martins, R. N. 2007. The role of presenilin and its interacting proteins in the biogenesis of Alzheimer's beta amyloid. Neurochem. Res. 32:609-623.
Vijg, J. 2008. The role of DNA damage and repair in aging: New approaches to an old problem. Mech. Ageing Dev. 129:498-502.
West, R. L., Lee, J. M., and Maroun, L. E. 1995. Hypomethylation of the amyloid precursor protein gene in the brain of an Alzheimer's disease patient. J. Mol. Neurosci. 6:141-146.
Wilson, V. L., and Jones, P. A. 1983. DNA methylation decreases in aging but not in immortal cells. Science 220:1055-1057.
Wilson, V. L., Smith, R. A., Ma, S., and Cutler, R. G. 1987. Genomic 5-methyldeoxycytidine decreases with age. J. Biol. Chem. 262:9948-9951.
Wilson, A. S., Power, B. E., and Molloy, P. L. 2007. DNA hypomethylation and human diseases. Biochim. Biophys. Acta 1775:138-162.
Wolfe, M. S. 2007. When loss is gain: reduced presenilin proteolytic function leads to increased Aβ42/Aβ40. EMBO Reports 8:136-140.
Worthley, S. G., Osende, J. I., Helft, G., Badimon, J. J., and Fuster, V. 2001. Coronary artery disease: pathogenesis and acute coronary syndromes. Mt. Sinai J. Med. 68:167-181.
Wu, H., Chen, Y., Liang, J., Shi, B., Wu G., Zhang, Y., Wang, D., Li, R., Yi, X., Zhang, H., Sun, L., and Shang, Y. 2005. Hypomethylation-linked activation of PAX2 mediates tamoxifenstimulated endometrial carcinogenesis. Nature 438:981-987.
Yenbutr, P., Hilakivi-Clarke, L., and Passaniti, A. 1998. Hypomethylation of an exon I estrogen receptor CpG island in spontaneous and carcinogen-induced mammary tumorigenesis in the rat. Mech. Ageing Dev. 106:93-102.
Yi, P., Melnyk, S., Pogribna, M., Pogribny, I. P., Hine, R. J., and James, S. J. 2000. Increase in plasma homocysteine associated with parallel increases in plasma S-adenosylhomocysteine and lymphocyte DNA hypomethylation. J. Biol. Chem. 275: 29318-29323.
Yideng, J., Jianzhong, Z., Ying, H., Juan, S., Jinge, Z., Shenglan, W., Xiaoqun, H., and Shuren, W. 2007. Homocysteine-mediated expression of SAHH, DMNTs, MBD2, and DNA hypomethylation potential pathogenic mechanism in VSMCs. DNA Cell. Biol. 26:603-611.
Yoder, J. A., Walsh, C. P., and Bestor, T. H. 1997. Cytosine methylation and the ecology of intragenomic parasites. Trends Genet. 13:335-340.
Yung, R. L., and Julius, A. 2008. Epigenetics, aging, and autoimmunity. Autoimmunity 41: 329-335.
Zaina, S., Lindholm, M. W., and Lund, G. 2005. Nutrition and aberrant DNA methylation patterns in atherosclerosis: more than just hyperhomocysteinemia? J. Nutr. 135:5-8.
Zhang, Z., Deng, C., Lu, Q., and Richardson, B. 2002. Age-dependent DNA methylation changes in the ITGAL (CD11a) promoter. Mech. Ageing Dev. 123:1257-1268.
Zhao, L., and Funk, C. D. 2004. Lipoxygenase pathways in atherosclerosis. Trends Cardiovasc. Med. 14:191-195.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

1. A method for producing a cell exhibiting one or more chronological marker, said method comprising: reducing the level of genomic nucleic acid methylation in a cell that is deficient in said one or more chronological marker in an amount and for a period of time sufficient to induce the production of said one or more chronological marker.

2. The method of claim 1 wherein said cell that is deficient in said one or more chronological marker is a stem cell or a somatic cell.

3. The method of claim 2 wherein said somatic cell is produced by a method comprising contacting a stem cell with one or more differentiation factors, wherein said differentiation factors promote the differentiation of said stem cell into said somatic cell.

4. The method of claim 3 wherein said stem cell is an induced pluripotent stem cell (iPSC) or embryonic stem cell (ESC).

5. The method of claim 4 wherein said somatic cell is selected from the group consisting of a fibroblast cell, a liver cell, a heart cell, a central nervous system (CNS) cell, a peripheral nervous system (PNS) cell, a kidney cell, a lung cell, a hematopoietic cell, a pancreatic beta cell, a bone marrow cell, an osteoblast cell, an osteoclast cell, an endothelial cell, a neural progenitor, a neuron, a glial cell, and a midbrain dopamine (mDA) neuron cell.

6. The method of claim 1 wherein said method of reducing genomic nucleic acid methylation comprises contacting the cell with an agent that inhibits nucleic acid methylation selected from the group consisting of a nucleoside analog of cytidine; 1-((3-D-Ribofuranosyl)-2(1H)-pyrimidinone; a DNA methyltransferase (DNMT) inhibitor; an inhibitor of histone methyltransferase (HMT); an inhibitor of methyl-CpG-binding protein (MeCP2); an inhibitor of a PHD and RING finger domains 1 protein (UHRF1); 5-aza-2-deoxycytidine (5-aza-dC), homocysteine; S-adenosyl-1-homocysteine (SAH); 4-Chloro-N-(4-hydroxy-1-naphthalenyl)-3-nitro-benzenesulfonamide; (3S,3'S,5aR,5aR,10bR,10′bR,11aS,11′aS)-2,2′,3,3′,5a,5′a,6,6′-octahydro-3,3′-bis(hydroxymethyl)-2,2′-dimethyl-[10b,10′b(11H,11′H)-bi3,11a-epidithio-11aH-pyrazino[1′,2′:1,5]pyrrolo[2,3-b]-indole]-1,1′,4,4′-tetrone; and combinations thereof.

7. The method of claim 6 wherein the DNA methyltransferase (DNMT) inhibitor, inhibitor of histone methyltransferase (HMT), inhibitor of methyl-CpG-binding protein (MeCP2), or inhibitor of a PHD and RING finger domains 1 protein (UHRF1) is selected from the group consisting of an antisense molecule; siRNA molecule; antibody or fragment thereof that specifically binds to the DNMT, HMT, MeCP2, UHRF1 or combinations thereof; and combinations thereof.

8. The method of claim 1 wherein said one or more chronological marker is selected from the group consisting of an age-associated marker, a maturation-associated marker, and a disease-associated marker.

9. A cell exhibiting at least one chronological marker induced by reducing the level of genomic nucleic acid methylation in a cell in an amount and for a period of time sufficient to induce said at least one chronological marker.

10. The cell of claim 9 wherein the level of genomic nucleic acid methylation is reduced to a level between 10 and 30% of a cell not expressing at least one age-associated chronological marker selected from Table 2 or Table 3, or wherein the level of genomic nucleic acid methylation is reduced by an amount of between 10 and 30% from the level of genomic nucleic acid methylation in a cell not expressing at least one age-associated chronological marker selected from Table 2 or Table 3.

11. The cell according to claim 9 wherein said cell is a somatic cell selected from the group consisting of a fibroblast cell, a liver cell, a heart cell, a CNS cell, a PNS cell, a kidney cell, a lung cell, a hematopoietic cell, a pancreatic beta cell, a bone marrow cell, an osteoblast cell, an osteoclast cell, an endothelial cell, a neural progenitor, a neuron, a glial cell, and a midbrain dopamine (mDA) neuron cell.

12. The cell according to claim 9 wherein said at least one chronological marker is selected from the group consisting of an age-associated marker, a maturation-associated marker, and a disease-associated marker.

13. A method for drug screening, said method comprising
contacting an age-modified cell with a candidate compound, wherein said age-modified cell exhibits at least one chronological marker induced by reducing the level of genomic nucleic acid methylation in the cell in an amount and for a period of time sufficient to induce said at least one chronological marker in said cell;
detecting an alteration in at least one of the survival, biological activity, morphology or structure of the cell; and
selecting as the drug a candidate compound that alters at least one of the survival, biological activity, morphology or structure of the cell.

14. A method for reducing the expression level of at least one chronological marker in a cell, said method comprising: increasing the level of genomic nucleic acid methylation in a cell expressing one or more chronological markers in an amount and for a period of time sufficient to reduce the expression level of said at least one chronological marker.

15. The method of claim 14 wherein said method of increasing the level of genomic nucleic acid methylation comprises contacting the cell with an agent that increases nucleic acid methylation selected from the group consisting of a PIWI protein, a PIWI-interacting RNA molecule, a DNA methyltransferase (DNMT) protein, a histone methyltransferase (HMT) protein, a methyl-CpG-binding protein (MeCP2), a PHD and RING finger domains 1 protein (UHRF1), resveratrol, rapamycin, and combination thereof.

16. A cell prepared by increasing the level of genomic nucleic acid methylation in the cell, wherein the level of methylation is increased in an amount and for a period of time sufficient to reduce expression of at least one chronological marker.

17. The cell of claim 16, wherein increasing genomic nucleic acid methylation comprises contacting the cell with an agent that increases nucleic acid methylation selected from the group consisting of a PIWI protein, a PIWI-interacting RNA molecule, a DNA methyltransferase (DNMT) protein, a histone methyltransferase (HMT) protein, a methyl-CpG-binding protein (MeCP2), a PHD and RING finger domains 1 protein (UHRF1), resveratrol, rapamycin, and combination thereof.

18. The method of claim 1, wherein the reduction in the level of genomic nucleic acid methylation comprises a reduction of methylation at non-coding regions of genomic nucleic acid repetitive elements.

19. The method of claim 18, wherein the repetitive elements are selected from the group consisting of LINE1 (L1) elements, LTR elements, Endogenous Retroviruses (ERV) elements, and combinations thereof.

20. The method of claim 18, wherein between about 20 and 80% of the repetitive elements in the cell exhibiting one or more chronological marker are hypomethylated compared to a cell that is deficient in said one or more chronological marker.

21. A method for determining the molecular age of a cell comprising determining the ratio of expression levels of one or more Line1 (L1), LTR, and/or ERV repetitive elements to one or more ALU repetitive elements in the cell, wherein a ratio greater than 1 is indicative of the cell having an aged or old molecular status.

22. A kit for producing a cell exhibiting one or more chronological marker, said kit comprising an agent that reduces genomic nucleic acid methylation in a cell.
